(19) United States
(12) Patent Application Publication
(10) Pub. No.: US 2026/0199239 A1
(43) Pub. Date: Jul. 16, 2026

(54) IONIZABLE LIPIDS WITH MALONATE TAILS

(71) Applicant: ModernaTX, Inc., Cambridge, MA (US)

(72) Inventor: Edward J. HENNESSY, Cambridge, MA (US)

(21) Appl. No.: 19/136,187

(22) PCT Filed: Dec. 7, 2023
(86) PCT No.: PCT/US2023/082851

§ 371 Date: Jun. 5, 2025

Related U.S. Application Data
(60) Provisional application No. 63/386,580, filed on Dec. 8, 2022.

(51) Int. Cl. A61K 9/1271; A61K 9/51; A61K 31/7105; C07C 229/24; C07C 233/36; C07C 233/62; C12N 15/88

(52) U.S. Cl. CPC A61K 9/1271; A61K 9/5123; A61K 31/7105; C07C 229/24; C07C 233/36; C07C 233/62; C12N 15/88; C07C 2601/04

(57) ABSTRACT

The present disclosure provides new ionizable lipids, lipid nanoparticle (LNP) compositions comprising the same, and methods of delivering therapeutic and/or prophylactic agents (e.g., mRNA) with the LNPs described herein. The ionizable lipids described herein comprise a central nitrogen atom bonded to (i) a head group; and (ii) two biodegradable, hydrophobic lipids tails, wherein at least one biodegradable lipid tail comprises a malonate moiety. Ionizable lipids described herein include compounds of Formula (I) and pharmaceutically acceptable salts thereof.

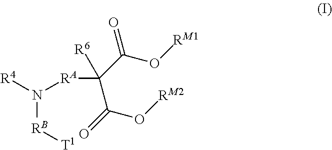

### RELATED APPLICATIONS

This application claims priority under 35 U.S.C. § 119(e) to United States Provisional Patent Application, U.S. Ser. No. 63/386,580, filed Dec. 8, 2022, the entire contents of which is incorporated herein by reference.

### BACKGROUND

The effective, targeted delivery of biologically active agents such as small molecule drugs, proteins, and nucleic acids represents a continuing medical challenge. In particular, the delivery of nucleic acids (e.g., mRNA) to cells is made difficult by the relative instability and low cell permeability of such agents. Thus, improved compositions and methods are needed to facilitate the delivery of therapeutic and/or prophylactic agents such as nucleic acids (e.g., mRNA) to cells.

Lipid nanoparticle (LNP), liposome, and lipoplex compositions have proven effective as transport vehicles to deliver biologically active substances such as small molecule drugs, proteins, and nucleic acids into cells and/or intracellular compartments. Such compositions can include one or more “cationic” lipids (i.e., ionizable lipids), phospholipids, structural lipids (e.g., sterols), and/or lipids containing polyethylene glycol (PEG lipids). Cationic lipids (i.e., ionizable lipids) include, for example, amine-containing lipids (i.e., amino lipids) that can be readily protonated under physiological conditions. Though a variety of such lipid-containing nanoparticle compositions have been reported, improvements in safety, efficacy, and specificity are sought.

### SUMMARY

Examples of cationic/ionizable amino lipids can be found in, e.g., International PCT Application Publication Nos. WO 2017/049245, published Mar. 23, 2017; WO 2017/112865, published Jun. 29, 2017; WO 2018/170306, published Sep. 20, 2018; WO 2018/232120, published Dec. 20, 2018; WO 2020/061367, published Mar. 26, 2020; WO 2021/055835, published Mar. 25, 2021; WO 2021/055833, published Mar. 25, 2021; WO 2021/055849, published Mar. 25, 2021; and WO 2022/204288, published Sep. 29, 2022, the entire contents of each of which is incorporated herein by reference.

The present disclosure provides new ionizable lipids, lipid nanoparticle (LNP) compositions comprising the same, and methods of delivering therapeutic and/or prophylactic agents (e.g., mRNA) with the LNPs described herein. The ionizable lipids described herein comprise a central nitrogen atom bonded to (i) a head group (e.g., R⁴); and (ii) two biodegradable, hydrophobic lipids tails, wherein at least one biodegradable lipid tail comprises a malonate moiety.

For example, in one aspect, provided herein are compounds of Formula (I):

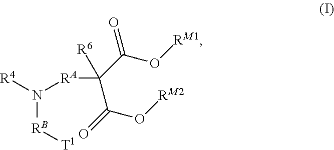

and pharmaceutically acceptable salts thereof, wherein:

T¹ is selected from:

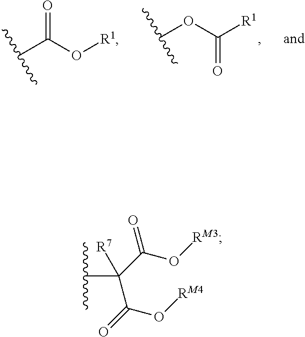

and wherein R^{A}, R^{B}, R¹, R⁴, R⁶, R⁷, R^{M1}, R^{M2}, R^{M3}, and R^{M4} are as defined herein.

In certain embodiments, the compound of Formula (I) is of Formula (I-a):

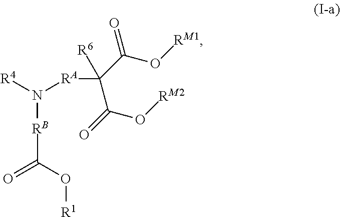

or a pharmaceutically acceptable salt thereof, wherein R^{A}, R^{B}, R¹, R⁴, R⁶, R⁷, R^{M1}, and R^{M2} are as defined herein.

In certain embodiments, the compound of Formula (I) is selected from the compounds provided in Table 1 (infra), and pharmaceutically acceptable salts thereof.

Also provided herein are lipid nanoparticles (LNPs) comprising an ionizable lipid described herein (e.g., a compound of Formula (I) or a pharmaceutically acceptable salt thereof). In certain embodiments, the LNP further comprises a phospholipid, a structural lipid, and a PEG lipid. In certain embodiments, the LNP comprises (e.g., encapsulates) an active agent such as a nucleic acid (e.g., mRNA). Pharmaceutical compositions comprising one or more LNPs described herein are also provided.

Methods are further provided herein, including but not limited to:
(a) Methods of delivering (e.g., specifically delivering) an active agent (e.g., nucleic acid, mRNA) to a cell of a subject comprising administering to the subject an LNP provided herein, wherein the LNP comprises the active agent.
(b) Methods of delivering (e.g., specifically delivering) an active agent (e.g., nucleic acid, mRNA) to an organ of a subject comprising administering to the subject an LNP provided herein, wherein the LNP comprises the active agent.
(c) Method of expressing a polypeptide in a cell of a subject comprising administering to the subject an LNP provided herein, wherein the LNP comprises an mRNA encoding the polypeptide.
(d) Methods of treating and/or preventing a disease in a subject in need thereof comprising administering to the subject an LNP provided herein.

The disclosure also includes methods of synthesizing ionizable lipids (e.g., a compound of Formula (I), (I-a), (I-b), (I-c), (I-d), (I-e), or (I-f)) described herein, and methods of making LNPs including a lipid component comprising an ionizable lipid described herein (e.g., a compound of Formula (I), (I-a), (I-b), (I-c), (I-d), (I-e), or (I-f)).

The details of certain embodiments of the disclosure are set forth in the Detailed Description of Certain Embodiments, as described below. Other features, objects, and advantages of the disclosure will be apparent from the Definitions, Examples, Figures, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms (“isotopically labeled derivatives”). For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain (i.e., unbranched) or branched saturated hydrocarbon group having a specified number of carbon atoms, for example, from 1 to 40 carbon atoms (“C₁₋₄₀ alkyl”). An alkyl group is branched or unbranched unless otherwise indicated. In some embodiments, an alkyl group has from 1 to 30 carbon atoms (“C₁₋₃₀ alkyl”). In some embodiments, an alkyl group has from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFC₁₂, —CF₂Cl, and the like.

The term “heteroalkyl” refers to an alkyl group, which further includes at least one heteroatom (e.g., 1, 2, 3, or 4 heteroatoms) selected from oxygen, nitrogen, sulfur, silicon, boron, and phosphorous within (e.g., inserted between adjacent carbon atoms of) and/or placed at one or more terminal position(s) of the parent chain. In certain embodiments, the heteroalkyl group is an alkyl group, which further includes at least one heteroatom (e.g., 1, 2, 3, or 4 heteroatoms) selected from oxygen, nitrogen, and sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“C₁₋₂₀ heteroalkyl”). In certain embodiments, a heteroalkyl group refers to a saturated group having from 1 to 12 carbon atoms and 1 or more heteroatoms within the parent chain (“C₁₋₁₂ heteroalkyl”). In some embodiments, a heteroalkyl group is a saturated group having 1 to 11 carbon atoms and 1 or more heteroatoms within the parent chain (“C₁₋₁₁ heteroalkyl”). In some embodiments, a heteroalkyl group is a saturated group having 1 to 10 carbon atoms and 1 or more heteroatoms within the parent chain (“C₁₋₁₀ heteroalkyl”). In some embodiments, a heteroalkyl group is a saturated group having 1 to 9 carbon atoms and 1 or more heteroatoms within the parent chain (“C₁₋₉ heteroalkyl”). In some embodiments, a heteroalkyl group is a saturated group having 1 to 8 carbon atoms and 1 or more heteroatoms within the parent chain (“C₁₋₈ heteroalkyl”). In some embodiments, a heteroalkyl group is a saturated group having 1 to 7 carbon atoms and 1 or more heteroatoms within the parent chain (“C₁₋₇ heteroalkyl”). In some embodiments, a heteroalkyl group is a saturated group having 1 to 6 carbon atoms and 1 or more heteroatoms within the parent chain (“C₁₋₆ heteroalkyl”). In some embodiments, a heteroalkyl group is a saturated group having 1 to 5 carbon atoms and 1 or 2 heteroatoms within the parent chain (“C₁₋₅ heteroalkyl”). In some embodiments, a heteroalkyl group is a saturated group having 1 to 4 carbon atoms and 1 or 2 heteroatoms within the parent chain (“C₁₋₄ heteroalkyl”). In some embodiments, a heteroalkyl group is a saturated group having 1 to 3 carbon atoms and 1 heteroatom within the parent chain (“C₁₋₃ heteroalkyl”). In some embodiments, a heteroalkyl group is a saturated group having 1 to 2 carbon atoms and 1 heteroatom within the parent chain (“C₁₋₂ heteroalkyl”). In some embodiments, a heteroalkyl group is a saturated group having 1 carbon atom and 1 heteroatom (“C₁ heteroalkyl”). In some embodiments, a heteroalkyl group is a saturated group having 2 to 6 carbon atoms and 1 or 2 heteroatoms within the parent chain (“C₂₋₆ heteroalkyl”). Unless otherwise specified, each instance of a heteroalkyl group is independently unsubstituted (an “unsubstituted heteroalkyl”) or substituted (a “substituted heteroalkyl”) with one or more substituents.

The term “alkenyl” refers to a radical of a straight-chain (i.e., unbranched) or branched hydrocarbon group having a specified number of carbon atoms, for example from 2 to 40 carbon atoms (“C₂₋₄₀ alkenyl”), and one or more carbon-carbon double bonds (e.g., 1, 2, 3, or 4 double bonds). An alkenyl group is branched or unbranched unless otherwise indicated. In some embodiments, an alkenyl group has 2 to 30 carbon atoms (“C₂₋₃₀ alkenyl”). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atom (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In an alkenyl group, a C═C double bond for which the stereochemistry is not specified (e.g., —CH═CHCH₃ or

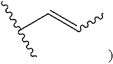

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, sulfur, silicon, boron, and phosphorous within (e.g., inserted between adjacent carbon atoms of) and/or placed at one or more terminal position(s) of the parent chain. In certain embodiments, the heteroalkenyl group is an alkenyl group, which further includes at least one heteroatom (e.g., 1, 2, 3, or 4 heteroatoms) selected from oxygen, nitrogen, and sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“C₂₋₂₀ heteroalkenyl”). In certain embodiments, a heteroalkenyl group refers to a group having from 2 to 12 carbon atoms, at least one double bond, and 1 or more heteroatoms within the parent chain (“C₂₋₁₂ heteroalkenyl”). In certain embodiments, a heteroalkenyl group refers to a group having from 2 to 11 carbon atoms, at least one double bond, and 1 or more heteroatoms within the parent chain (“C₂₋₁₁ heteroalkenyl”). In certain embodiments, a heteroalkenyl group refers to a group having from 2 to 10 carbon atoms, at least one double bond, and 1 or more heteroatoms within the parent chain (“C₂₋₁₀ heteroalkenyl”). In some embodiments, a heteroalkenyl group has 2 to 9 carbon atoms at least one double bond, and 1 or more heteroatoms within the parent chain (“C₂₋₉ heteroalkenyl”). In some embodiments, a heteroalkenyl group has 2 to 8 carbon atoms, at least one double bond, and 1 or more heteroatoms within the parent chain (“C₂₋₈ heteroalkenyl”). In some embodiments, a heteroalkenyl group has 2 to 7 carbon atoms, at least one double bond, and 1 or more heteroatoms within the parent chain (“C₂₋₇ heteroalkenyl”). In some embodiments, a heteroalkenyl group has 2 to 6 carbon atoms, at least one double bond, and 1 or more heteroatoms within the parent chain (“C₂₋₆ heteroalkenyl”). In some embodiments, a heteroalkenyl group has 2 to 5 carbon atoms, at least one double bond, and 1 or 2 heteroatoms within the parent chain (“C₂₋₅ heteroalkenyl”). In some embodiments, a heteroalkenyl group has 2 to 4 carbon atoms, at least one double bond, and 1 or 2 heteroatoms within the parent chain (“C₂₋₄ heteroalkenyl”). In some embodiments, a heteroalkenyl group has 2 to 3 carbon atoms, at least one double bond, and 1 heteroatom within the parent chain (“C₂₋₃ heteroalkenyl”). In some embodiments, a heteroalkenyl group has 2 carbon atoms, at least one double bond, and 1 heteroatom within the parent chain (“C₂ heteroalkenyl”). In some embodiments, a heteroalkenyl group has 2 to 6 carbon atoms, at least one double bond, and 1 or 2 heteroatoms within the parent chain (“C₂₋₆ heteroalkenyl”). Unless otherwise specified, each instance of a heteroalkenyl group is independently unsubstituted (an “unsubstituted heteroalkenyl”) or substituted (a “substituted heteroalkenyl”) with one or more substituents.

The term “alkynyl” refers to a radical of a straight-chain (i.e., unbranched) or branched hydrocarbon group having a specified number of carbon atoms, for example from 2 to 40 carbon atoms (“C₂₋₄₀ alkynyl”), and one or more carbon-carbon triple bonds (e.g., 1, 2, 3, or 4 triple bonds). An alkynyl group is branched or unbranched unless otherwise indicated. In some embodiments, an alkynyl group has 2 to 30 carbon atoms (“C₂₋₃₀ alkynyl”). In some embodiments, an alkynyl group has 2 to 20 carbon atoms (“C₂₋₃₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents.

The term “heteroalkynyl” refers to an alkynyl group, which further includes at least one heteroatom (e.g., 1, 2, 3, or 4 heteroatoms) selected from oxygen, nitrogen, sulfur, silicon, boron, and phosphorous within (e.g., inserted between adjacent carbon atoms of) and/or placed at one or more terminal position(s) of the parent chain. In certain embodiments, the heteroalkynyl group is an alkynyl group, which further includes at least one heteroatom (e.g., 1, 2, 3, or 4 heteroatoms) selected from oxygen, nitrogen, and sulfur within (e.g., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“C₂₋₂₀ heteroalkynyl”). In certain embodiments, a heteroalkynyl group refers to a group having from 2 to 10 carbon atoms, at least one triple bond, and 1 or more heteroatoms within the parent chain (“C₂₋₁₀ heteroalkynyl”). In some embodiments, a heteroalkynyl group has 2 to 9 carbon atoms, at least one triple bond, and 1 or more heteroatoms within the parent chain (“C₂₋₉ heteroalkynyl”). In some embodiments, a heteroalkynyl group has 2 to 8 carbon atoms, at least one triple bond, and 1 or more heteroatoms within the parent chain (“C₂₋₈ heteroalkynyl”). In some embodiments, a heteroalkynyl group has 2 to 7 carbon atoms, at least one triple bond, and 1 or more heteroatoms within the parent chain (“C₂₋₇ heteroalkynyl”). In some embodiments, a heteroalkynyl group has 2 to 6 carbon atoms, at least one triple bond, and 1 or more heteroatoms within the parent chain (“C₂₋₆ heteroalkynyl”). In some embodiments, a heteroalkynyl group has 2 to 5 carbon atoms, at least one triple bond, and 1 or 2 heteroatoms within the parent chain (“C₂₋₅ heteroalkynyl”). In some embodiments, a heteroalkynyl group has 2 to 4 carbon atoms, at least one triple bond, and 1 or 2 heteroatoms within the parent chain (“C₂₋₄ heteroalkynyl”). In some embodiments, a heteroalkynyl group has 2 to 3 carbon atoms, at least one triple bond, and 1 heteroatom within the parent chain (“C₂₋₃ heteroalkynyl”). In some embodiments, a heteroalkynyl group has 2 carbon atoms, at least one triple bond, and 1 heteroatom within the parent chain (“C₂ heteroalkynyl”). In some embodiments, a heteroalkynyl group has 2 to 6 carbon atoms, at least one triple bond, and 1 or 2 heteroatoms within the parent chain (“C₁₋₆ heteroalkynyl”). Unless otherwise specified, each instance of a heteroalkynyl group is independently unsubstituted (an “unsubstituted heteroalkynyl”) or substituted (a “substituted heteroalkynyl”) with one or more substituents.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl includes 0, 1, or 2 C═C double bonds in the carbocyclic ring system, as valency permits.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, sulfur, silicon, boron, and phosphorous (“3-14 membered heterocyclyl”). In certain embodiments, the heterocyclyl group is a radical of a 3- to 14-membered non-aromatic ring system having ring carbon atoms and 1 to 4 ring heteroatoms, wherein each heteroatom is independently selected from nitrogen, oxygen, and sulfur. The point of attachment can be either to a ring carbon atom or a ring heteroatom of the heterocyclyl group, as valency permits. For example, in heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl is substituted or unsubstituted, 3- to 8-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzo-thienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydro-pyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, sulfur, silicon, boron, and phosphorous (“5-14 membered heteroaryl”). In certain embodiments, the heteroaryl group is a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur. The point of attachment can be either to a ring carbon atom or a ring heteroatom of the heteroaryl group, as valency permits. For example, in heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

The term “halo” or “halogen” refers to fluorine (fluoro, —F), chlorine (chloro, —Cl), bromine (bromo, —Br), or iodine (iodo, —I).

The term “silyl” refers to the group —Si(R^{aa})₃, wherein R^{aa} is as defined herein.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present disclosure contemplates any and all such combinations in order to arrive at a stable compound. For purposes of this disclosure, heteroatoms such as nitrogen, oxygen, and sulfur may have hydrogen substituents and/or any suitable substituent as described herein which satisfy the valencies of the heteroatoms and results in the formation of a stable moiety. The embodiments described herein are not limited in any manner by the exemplary substituents described herein.

In certain embodiments, exemplary substituents (e.g., carbon atom substituents) are selected from halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR)₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O) SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR)₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR)₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₂₋₂₀ alkenyl, C₂₋₂₀ alkynyl, C₁₋₂₀ heteroalkyl, C₂₋₂₀ heteroalkenyl, C₂₋₂₀ heteroalkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₂₋₂₀ alkenyl, C₂₋₂₀ alkynyl, C₁₋₂₀ heteroalkyl, C₂₋₂₀ heteroalkenyl, C₂₋₂₀ heteroalkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR)₂, —P(═O)(N(R^{cc})₂)₂, C₁₋₂₀ alkyl, C₁₋₂₀ perhaloalkyl, C₂₋₂₀ alkenyl, C₂₋₂₀ alkynyl, C₁₋₂₀ heteroalkyl, C₂₋₂₀ heteroalkenyl, C₂₋₂₀ heteroalkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₂₋₂₀ alkenyl, C₂₋₂₀ alkynyl, C₁₋₂₀ heteroalkyl, C₂₋₂₀ heteroalkenyl, C₂₋₂₀ heteroalkynyl, C₃₋₁₀ carbocyclyl, 3-14 membered heterocyclyl, C₆₋₁₄ aryl, and 5-14 membered heteroaryl, or two Rec groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O) SR^{ee}, —C(═S) SR^{ee}, —SC(═S) SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S;
each instance of R^{ee} is, independently, selected from C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, selected from halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂ (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH) (C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, C₆₋₁₀ aryl, 3-10 membered heterocyclyl, and 5-10 membered heteroaryl; or two geminal R^{gg} substituents are joined to form ═O or ═S; and
each X⁻ is independently a counterion.

In certain embodiments, the molecular weight of a substituent (e.g., carbon atom substituent) is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms.

In certain embodiments, exemplary substituents (e.g., carbon atom substituents) are selected from halogen, —CN, —NO₂, —N₃, —SO₂H, —SO₃H, —OH, —OR^{aa}, —N(R^{bb})₂, —N(R^{bb})₃⁺X⁻, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-14 membered heterocyclyl, C₆₋₁₄ aryl, and 5-14 membered heteroaryl; wherein X⁻ is a counterion;
or two geminal hydrogens on a carbon atom are replaced with the group ═O, ═S, ═NN(R^{bb})₂, ═NNR^{bb}C(═O)R^{aa}, —NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring; and
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-14 membered heterocyclyl, C₆₋₁₄ aryl, and 5-14 membered heteroaryl, or two Rec groups are joined to form a 3-14 membered heterocyclyl or 5-14 membered heteroaryl ring.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, or —NO₂. In certain embodiments, each carbon atom substituent is independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include-OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, Rec and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that includes the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2¢- and 4¢-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di (2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that includes the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4¢,8¢-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl (pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds). In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include-R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb}) OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and Rec are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di (p-methoxyphenyl)phenylmethyl, tri (p-methoxyphenyl)methyl, 4-(4′-bromophenacyloxyphenyl)diphenylmethyl, 4,4′,4″-tris (4,5-dichlorophthalimidophenyl)methyl, 4,4′,4″-tris (levulinoyloxyphenyl)methyl, 4,4′,4″-tris (benzoyloxyphenyl)methyl, 4,4′-Dimethoxy-3′″-[N-(imidazolylmethyl)]trityl Ether (IDTr-OR), 4,4′-Dimethoxy-3″-[N-(imidazolylethyl)carbamoyl]trityl Ether (IETr-OR), 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv). In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O) SR^{aa}, —C(═O)R^{aa}, —CO₂R^{aa}, —C(═O)N(R^{bb})₂, —C(═NR^{bb})R^{aa}, —C(═NR^{bb}) OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and Rec are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Lipids of the disclosure that contain nitrogen atoms (e.g., compounds of Formula (I)) can be converted to N-oxides by treatment with an oxidizing agent (e.g., 3-chloroperoxybenzoic acid (mCPBA) and/or hydrogen peroxides) to afford other lipids of the disclosure. Thus, all shown and claimed nitrogen-containing lipids are considered, when allowed by valency and structure, to include both the lipid as shown and its N-oxide derivative (which can be designated as N—O or N⁺—O⁻). Furthermore, in other instances, the nitrogens in the lipids of the disclosure can be converted to N-hydroxy or N-alkoxy lipids. For example, N-hydroxy lipids can be prepared by oxidation of the parent amine by an oxidizing agent such as m-CPBA. All shown and claimed nitrogen-containing lipids are also considered, when allowed by valency and structure, to cover both the lipid as shown and its N-hydroxy (i.e., N—OH) and N-alkoxy (i.e., N—OR′, wherein R′ is substituted or unsubstituted C₁₋₆ alkyl, C₂₋₆ alkenyl, C₂₋₆ alkynyl, C₃₋₈ carbocycle or 3-8 membered heterocycle) derivatives.

These and other exemplary substituents are described in more detail in the Detailed Description, Examples, Figures, and Claims. The embodiments provided herein are not limited in any manner by the above exemplary listing of substituents.

### Other Definitions

The following definitions are more general terms used throughout the present application.

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given lipid in a lipid component of a nanoparticle composition, “about” may mean+/−10% of the recited value. For instance, a nanoparticle composition including a lipid component having about 40% of a given lipid may include 30-50% of the lipid.

The term “biodegradable” refers to the ability of a compound or chemical moiety to be broken down and/or metabolized under physiological conditions. For example, lipids comprising ester groups are considered biodegradable due to their ability to be hydrolyzed by esterases, resulting in alcohol and carboxylic acid metabolites. Malonates are examples of biodegradable groups as they can be broken down and/or metabolized under physiological conditions.

The terms “composition” and “formulation” are used interchangeably.

Throughout the present disclosure, references to “the compound,” “a compound,” “the lipid,” “a lipid,” “the ionizable lipid,” “a ionizable lipid” (and the like) provided herein are intended to encompass the compound or group of compounds (or lipid or group of lipids), and also pharmaceutically acceptable salts, isomers (e.g., stereoisomers), tautomers, isotopically labeled derivatives, solvates, and hydrates thereof as described herein.

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of lipid nanoparticles (e.g., empty LNPs or loaded LNPs). Moreover, more than one mammalian cell may be contacted by a nanoparticle composition.

As used herein, the term “delivering” means providing an entity to a destination. For example, delivering a therapeutic and/or prophylactic agent to a subject may involve administering a nanoparticle composition including the therapeutic and/or prophylactic agent to the subject (e.g., by an intravenous, intramuscular, intradermal, or subcutaneous route). Administration of a nanoparticle composition to a mammal or mammalian cell may involve contacting one or more cells with the nanoparticle composition.

As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic agent by a nanoparticle to a cell or target tissue of interest (e.g., mammalian liver) compared to the level of delivery of a therapeutic and/or prophylactic agent by a control nanoparticle to a cell or target tissue of interest (e.g., a nanoparticle containing MC3, KC2, or DLinDMA). The level of delivery of a nanoparticle to a particular tissue may be measured by comparing the amount of protein produced in a tissue to the weight of said tissue, comparing the amount of therapeutic and/or prophylactic agent in a tissue to the weight of said tissue, comparing the amount of protein produced in a tissue to the amount of total protein in said tissue, or comparing the amount of therapeutic and/or prophylactic agent in a tissue to the amount of total therapeutic and/or prophylactic agent in said tissue. It will be understood that the enhanced delivery of a nanoparticle to a cell or target tissue need not be determined in a subject being treated, it may be determined in a surrogate such as an animal model (e.g., a rat model). In certain embodiments, a nanoparticle composition including an ionizable lipid (e.g., a compound of Formula (I), (I-a), (I-b), (I-c), (I-d), (I-e), or (I-f)) has substantively the same level of delivery enhancement regardless of administration routes. For example, certain lipids disclosed herein exhibit similar delivery enhancement when they are used for delivering a therapeutic and/or prophylactic agent either intravenously or intramuscularly. In other embodiments, certain lipids disclosed herein exhibit a higher level of delivery enhancement when they are used for delivering a therapeutic and/or prophylactic agent intramuscularly than intravenously.

As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic agent by a nanoparticle to a cell or target tissue of interest (e.g., mammalian liver) compared to an off-target cell or tissue (e.g., mammalian spleen). The level of delivery of a nanoparticle to a particular tissue may be measured by comparing the amount of protein produced in a tissue to the weight of said tissue, comparing the amount of therapeutic and/or prophylactic agent in a tissue to the weight of said tissue, comparing the amount of protein produced in a tissue to the amount of total protein in said tissue, or comparing the amount of therapeutic and/or prophylactic agent in a tissue to the amount of total therapeutic and/or prophylactic agent in said tissue. For example, for renovascular targeting, a therapeutic and/or prophylactic agent is specifically provided to a mammalian kidney as compared to the liver and spleen if 1.5, 2-fold, 3-fold, 5-fold, 10-fold, 15 fold, or 20 fold more therapeutic and/or prophylactic agent per 1 g of tissue is delivered to a kidney compared to that delivered to the liver or spleen following systemic administration of the therapeutic and/or prophylactic agent. It will be understood that the ability of a nanoparticle to specifically deliver to a target tissue need not be determined in a subject being treated, it may be determined in a surrogate such as an animal model (e.g., a rat model).

As used herein, “encapsulation efficiency” (EE) refers to the amount of a therapeutic and/or prophylactic agent that becomes part of a nanoparticle composition, relative to the initial total amount of therapeutic and/or prophylactic agent used in the preparation of a nanoparticle composition. For example, if 97 mg of therapeutic and/or prophylactic agent are encapsulated in a nanoparticle composition out of a total 100 mg of therapeutic and/or prophylactic agent initially provided to the composition, the encapsulation efficiency may be given as 97%.

As used herein, “encapsulation,” “encapsulated,” “loaded,” and “associated” may refer to complete, substantial, or partial enclosure, confinement, surrounding, or encasement. As used herein, “encapsulation” or “association” may refer to the process of confining an individual nucleic acid molecule within a nanoparticle and/or establishing a physiochemical relationship between an individual nucleic acid molecule and a nanoparticle.

As used herein, an “empty LNP” or “empty nanoparticle” may refer to a nanoparticle that is substantially free of a therapeutic or prophylactic agent. As used herein, an “empty nanoparticle” or an “empty lipid nanoparticle” may refer to a nanoparticle that is substantially free of a nucleic acid. As used herein, an “empty nanoparticle” or an “empty lipid nanoparticle” may refer to a nanoparticle that is substantially free of a nucleotide or a polypeptide. As used herein, an “empty nanoparticle” or an “empty lipid nanoparticle” may refer to a nanoparticle that consists substantially of only lipid components.

As used herein, a “loaded LNP” or “loaded nanoparticle” (also referred to as a “full nanoparticle” or a “full lipid nanoparticle”) may refer to a nanoparticle comprising the components of the empty nanoparticle, and a substantial amount of a therapeutic or prophylactic agent. In some embodiments, the loaded LNP comprises a therapeutic or prophylactic agent that is at least partially in the interior of the LNP. In some embodiments, the loaded LNP comprises a substantial amount of a therapeutic or prophylactic agent that is associated with the surface of the LNP or conjugated to the exterior of the LNP. As used herein, a “loaded LNP” or “loaded nanoparticle” (also referred to as a “full nanoparticle” or a “full lipid nanoparticle”) may refer to a nanoparticle comprising the components of the empty nanoparticle, and a substantial amount of a nucleotide or polypeptide. In some embodiments, the loaded LNP comprises a nucleotide or polypeptide that is at least partially in the interior of the LNP. In some embodiments, the loaded LNP comprises a nucleotide or polypeptide that is associated with the surface of the LNP or conjugated to the exterior of the LNP. As used herein, a “loaded LNP” or “loaded nanoparticle” (also referred to as a “full nanoparticle” or a “full lipid nanoparticle”) may refer to a nanoparticle comprising the components of the empty nanoparticle, and a substantial amount of a nucleic acid. In some embodiments, the loaded LNP comprises a nucleic acid (e.g., an mRNA) that is at least partially in the interior of the LNP. In some embodiments, the loaded LNP comprises nucleic acid (e.g., an mRNA) that is associated with the surface of the LNP or conjugated to the exterior of the LNP.

As used herein, “expression” of a nucleic acid sequence refers to translation of an mRNA into a polypeptide or protein and/or post-translational modification of a polypeptide or protein.

As used herein “hydrophobicity” of a lipid describes the tendency of a lipid to exclude water. In some embodiments, the hydrophobicity of a lipid nanoparticle surface impacts the penetration of a lipid nanoparticle across the lipid bilayer of a cell. In some embodiments, hydrophobic nanoparticles show increased cellular uptake relative to hydrophilic lipid nanoparticles.

As used herein, the term “in vitro” refers to events that occur in an artificial environment, e.g., in a test tube or reaction vessel, in cell culture, in a Petri dish, etc., rather than within an organism (e.g., animal, plant, or microbe). As used herein, the term “in vivo” refers to events that occur within an organism (e.g., animal, plant, or microbe or cell or tissue thereof). As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

As used herein, the term “isomer” means any geometric isomer, tautomer, zwitterion, or stereoisomer of a compound (e.g., a lipid of the disclosure). Compounds (e.g., lipids of the disclosure) may include one or more chiral centers and/or double bonds and may thus exist as stereoisomers, such as double-bond isomers (i.e., geometric E/Z isomers or cis/trans isomers), enantiomers, or diastereomers. The present disclosure encompasses any and all isomers of the lipids described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of lipids and means of resolving them into their component enantiomers or stereoisomers are well-known.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of tautomerism in di-substituted guanidine is shown below.

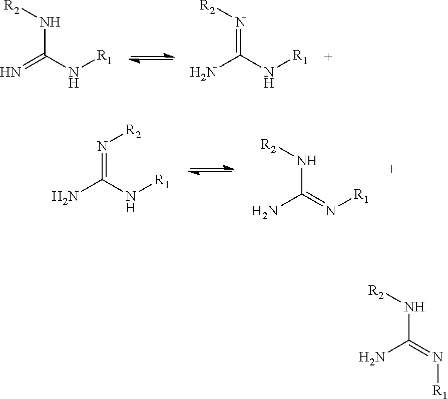

It is to be understood that the lipids of the disclosure may be depicted as different tautomers. It should also be understood that when lipids have tautomeric forms, all tautomeric forms are intended to be included in the scope of the disclosure, and the naming of the lipids does not exclude any tautomer form.

As used herein, a “lipid component” is that component of a nanoparticle composition that includes one or more lipids. For example, the lipid component may include one or more cationic/ionizable, PEGylated, structural, or other lipids, such as phospholipids.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. For example, two nucleosides of a cap analog may be linked at their 5′ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

As used herein, the “N: P ratio” is the molar ratio of ionizable (in the physiological pH range) nitrogen atoms in a lipid to phosphate groups in an RNA, e.g., in a nanoparticle composition including a lipid component and an RNA.

As used herein, a “nanoparticle composition” is a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less.

As used herein, “naturally occurring” means existing in nature without artificial aid.

As used herein, a “PEG lipid” or “PEGylated lipid” refers to a lipid comprising a polyethylene glycol (PEG) component.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers to any ingredient other than the lipids described herein (for example, a vehicle capable of suspending, complexing, or dissolving the active lipid) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

In the present specification, the structural formula of the lipid represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the lipids represented by the formula. It is noted that any crystal form, crystal form mixture, or anhydride or hydrate thereof is included in the scope of the present disclosure.

The term “crystal polymorphs,” “polymorphs” or “crystal forms” means crystal structures in which a compound (e.g., a lipid of the disclosure; or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the lipids can be prepared by crystallization under different conditions.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the “polydispersity index,” or “PDI” is a ratio that describes the homogeneity of the particle size distribution of a system. A small value, e.g., less than 0.3, indicates a narrow particle size distribution.

As used herein, the term “polypeptide” or “polypeptide of interest” refers to a polymer of amino acid residues typically joined by peptide bonds that can be produced naturally (e.g., isolated or purified) or synthetically. The terms “polypeptide,” “peptide,” and “protein” are used interchangeably herein to refer to polymers of amino acids of any length. The polymer can comprise natural and/or non-natural (e.g., modified) amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. The term polypeptide can also apply to amino acid polymers in which one or more amino acid residues are an artificial chemical analogue of a corresponding naturally occurring amino acid. The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. In some embodiments, a “peptide” can be less than or equal to 50 amino acids long, e.g., about 5, 10, 15, 20, 25, 30, 35, 40, 45, or 50 amino acids long.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. As used herein, a “DNA” refers to a desoxyribonucleic acid that may be naturally or non-naturally occurring. For example, a DNA may be a synthetic molecule, e.g., a synthetic DNA molecule produced in vitro. In some embodiments, the DNA molecule is a recombinant molecule. As used herein, a “recombinant DNA molecule” refers to a DNA molecule that does not exist as a natural product, but is produced using molecular biology techniques.

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+ (C₁₋₄ alkyl) 4″ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “single unit dose” is a dose of any therapeutic administered in one dose/at one time/single route/single point of contact, i.e., single administration event. As used herein, a “split dose” is the division of single unit dose or total daily dose into two or more doses.

As used herein, “size” or “mean size” in the context of lipid nanoparticles (e.g., empty LNPs or loaded LNPs) refers to the mean diameter of a nanoparticle composition.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates. The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula Rxx H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R×0.5 H₂O)), and polyhydrates (x is a number greater than 1, e.g., dihydrates (R×2 H₂O) and hexahydrates (R×6 H₂O)).

As used herein, the term “subject” refers to any organism to which a composition in accordance with the disclosure may be administered, e.g., for experimental, diagnostic, prophylactic, and/or therapeutic purposes. A subject to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. As used herein, “patient” refers to a subject who may seek or be in need of treatment, requires treatment, is receiving treatment, will receive treatment, or a subject who is under care by a trained professional for a particular disease or condition.

As used herein, a “total daily dose” is an amount given or prescribed in 24 hour period. It may be administered as a single unit dose.

As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient.

As used herein “target tissue” refers to any one or more tissue types of interest in which the delivery of a therapeutic and/or prophylactic agent would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, “transfection” refers to the introduction of a species (e.g., an RNA) into a cell. Transfection may occur, for example, in vitro, ex vivo, or in vivo.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. As used herein, the term “preventing,” “prevent,” or “protecting against” describes reducing or eliminating the onset of a disease, disorder or condition or reducing or eliminating the onset of symptoms or complications of such disease, disorder or condition. For example, “preventing” can be by means of a vaccine, whereby the vaccine can be used to prevent a disease, disorder or condition, e.g., prevent a viral infection.

As used herein, the “zeta potential” is the electrokinetic potential of a lipid, e.g., in a particle composition.

### BRIEF DESCRIPTION OF THE DRAWINGS

The accompanying drawings, which are incorporated in and constitute a part of this specification, illustrate several embodiments of the description and provide non-limiting examples of the subject matter described herein.

FIGS. 1-3. FIG. 1 In vivo hEPO protein expression at 6 hours with LNPs formulated with ionizable lipids M1-M3. FIG. 2 In vivo hEPO protein expression at 24 hours with LNPs formulated with ionizable lipids M1-M3. FIG. 3 shows a comparison of the 6 hour and 24 hour timepoints.

FIGS. 4-6. FIG. 4 In vivo hEPO protein expression at 6 hours with LNPs formulated with ionizable lipids M1 and M3-M6. FIG. 5 In vivo hEPO protein expression at 24 hours with LNPs formulated with ionizable lipids M1 and M3-M6. FIG. 6 shows a comparison of the 6 hour and 24 hour timepoints.

### DETAILED DESCRIPTION OF CERTAIN EMBODIMENTS

The present disclosure provides new ionizable lipids (e.g., a compound of Formula (I), (I-a), (I-b), (I-c), (I-d), (I-e), or (I-f)), lipid nanoparticle (LNP) compositions comprising the same, and methods of delivering therapeutic and/or prophylactic agents (e.g., mRNA) with the LNPs described herein. The ionizable lipids described herein comprise a central nitrogen atom bonded to (i) a head group (e.g., R⁴); and (ii) two biodegradable, hydrophobic lipids tails, wherein at least one biodegradable lipid tail comprises a malonate moiety.

### Malonate-Containing Lipids

In one aspect, provided herein are malonate-containing lipids such as compounds of Formula (I):

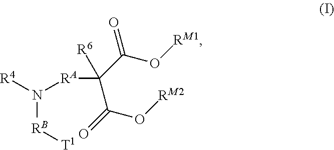

and pharmaceutically acceptable salts thereof, wherein:
T¹ is selected from:

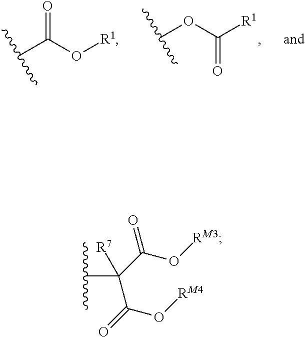

R^{A} and R^{B} are each independently optionally substituted C₄₋₂₀ alkylene, optionally substituted C₄₋₂₀ alkenylene, or optionally substituted C₄₋₂₀ alkynylene;
R¹ is optionally substituted, branched or unbranched C₁₋₃₀ alkyl, optionally substituted, branched or unbranched C₂₋₃₀ alkenyl, or optionally substituted, branched or unbranched C₂₋₃₀ alkynyl;
R^{M1}, R^{M2}, R^{M3}, and R^{M4} are each independently optionally substituted, branched or unbranched C₁₋₂₀ alkyl, optionally substituted, branched or unbranched C₂₋₂₀ alkenyl, or optionally substituted, branched or unbranched C₂₋₂₀ alkynyl;
R⁴ is a head group selected from optionally substituted C₁₋₆ alkyl, optionally substituted C₂₋₆ alkenyl, optionally substituted C₂₋₆ alkynyl, optionally substituted C₃₋₈ carbocyclyl, and —Y¹-Q;
Y¹ is optionally substituted C₁₋₁₀ alkylene, optionally substituted C₂₋₁₀ alkenylene, and optionally substituted C₂₋₁₀ alkynylene;
Q is optionally substituted C₃₋₈ carbocyclyl, optionally substituted C₆₋₁₀ aryl, optionally substituted C₃₋₈ heterocyclyl, optionally substituted C₅₋₁₀ heteroaryl, —OR^{O}, —O(CH₂)ₙN(RN)₂, —O(CH₂)ₙOR^{O}, —C(O)OR^{O}, —OC(O)R, —OC(O)OR^{O}, —CX₃, —CX₂H, —CXH₂, —CN, —N(RN)₂, —N(R^{N})(CH₂)ₙN(R^{N})₂, —N(R^{N})(CH₂)ₙOR^{O}, —C(O)N(R^{N})₂, —N(R^{N})C(O)R, —N(R^{N})C(O)N(R^{N})₂, —N(R^{N})C(S)N(R^{N})₂, —N(R^{N})C(═NR⁵)N(R^{N})₂, —N(R^{N})C(═CHR⁵)N(R^{N})₂, —C(═NR⁵)N(R^{N})₂, —C(═NR⁵)R, —OC(O)N(R^{N})₂, —N(R^{N})C(O)OR^{O}, —C(R)N(R^{N})₂C(O)OR^{O}, —N(R^{N})S(O)₂R, —N(R^{N})S(O)R, —S(O)₂N(R^{N})₂, —S(O)N(R^{N})₂,

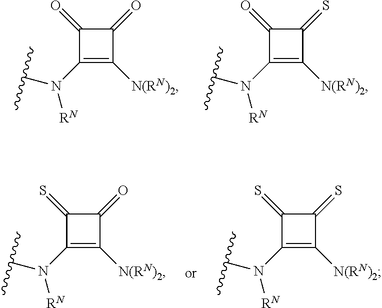

each R is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, or optionally substituted C₅₋₁₀ heteroaryl;
each R^{O} is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or an oxygen protecting group;
each R^{N} is independently hydrogen, —OR^{O}, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or a nitrogen protecting group, or two R^{N} attached to the same nitrogen atoms are joined together with the intervening atoms to form optionally substituted heterocyclyl or optionally substituted heteroaryl;
R⁵ is hydrogen, —CN, —NO₂, —OR^{O}, —S(O)₂R, —S(O)₂N(R^{N})₂, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, or optionally substituted C₁₋₆ acyl, or optionally a nitrogen protecting group when attached to a nitrogen atom;
R⁶ and R⁷ are each independently hydrogen, halogen, or optionally substituted C₁₋₆ alkyl;
each X is independently halogen; and
n is 1, 2, 3, 4, or 5.

In certain embodiments, the malonate-containing lipid is a compound of Formula (I):

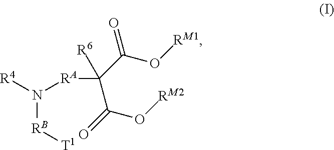

or a pharmaceutically acceptable salt thereof, wherein:
T¹ is selected from:

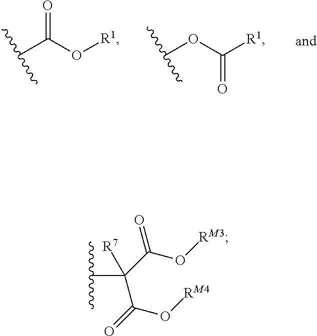

R^{A} and R^{B} are each independently optionally substituted C₁₋₂₀ alkylene, optionally substituted C₁₋₂₀ alkenylene, or optionally substituted C₁₋₂₀ alkynylene;
R¹ is optionally substituted, branched or unbranched C₁₋₃₀ alkyl, optionally substituted, branched or unbranched C₂₋₃₀ alkenyl, or optionally substituted, branched or unbranched C₂₋₃₀ alkynyl;
R^{M1}, R^{M2}, R^{M3}, and R^{M4} are each independently optionally substituted, branched or unbranched C₁₋₂₀ alkyl, optionally substituted, branched or unbranched C₂₋₂₀ alkenyl, or optionally substituted, branched or unbranched C₂₋₂₀ alkynyl;
R⁴ is a head group selected from optionally substituted C₁₋₆ alkyl, optionally substituted C₂₋₆ alkenyl, optionally substituted C₂₋₆ alkynyl, optionally substituted C₃₋₈ carbocyclyl, and —Y¹-Q;
Y¹ is optionally substituted C₁₋₁₀ alkylene, optionally substituted C₂₋₁₀ alkenylene, and optionally substituted C₂₋₁₀ alkynylene;
Q is optionally substituted C₃₋₈ carbocyclyl, optionally substituted C₆₋₁₀ aryl, optionally substituted C₃₋₈ heterocyclyl, optionally substituted C₅₋₁₀ heteroaryl, —OR^{O}, —O(CH₂)ₙN(R^{N})₂, —O(CH₂)ₙOR^{O}, —C(O)OR^{O}, —OC(O)R, —OC(O)OR^{O}, —CX₃, —CX₂H, —CXH₂, —CN, —N(R^{N})₂, —N(R^{N})(CH₂)ₙN(R^{N})₂, —N(R^{N})(CH₂)ₙOR^{O}, —C(O)N(R^{N})₂, —N(R^{N})C(O)R, —N(R^{N})C(O)N(R^{N})₂, —N(R^{N})C(S)N(R^{N})₂, —N(R^{N})C(═NR⁵)N(R^{N})₂, —N(R^{N})C(═CHR⁵)N(R^{N})₂, —C(═NR⁵)N(R^{N})₂, —C(═NR⁵)R, —OC(O)N(R^{N})₂, —N(R^{N})C(O)OR^{O}, —C(R)N(R^{N})₂C(O)OR^{O}, —N(R^{N})S(O)₂R, —N(R^{N})S(O)R, —S(O)₂N(R^{N})₂, —S(O)N(R^{N})₂,

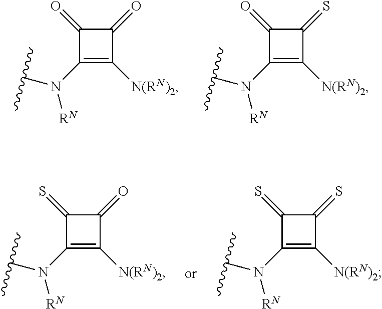

each R is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, or optionally substituted C₅₋₁₀ heteroaryl;
each R^{O} is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or an oxygen protecting group;
each R^{N} is independently hydrogen, —OR^{O}, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or a nitrogen protecting group, or two R^{N} attached to the same nitrogen atoms are joined together with the intervening atoms to form optionally substituted heterocyclyl or optionally substituted heteroaryl;
R⁵ is hydrogen, —CN, —NO₂, —OR^{O}, —S(O)₂R, —S(O)₂N(R^{N})₂, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, or optionally substituted C₅₋₁₀ heteroaryl, or optionally substituted C₁₋₆ acyl, or optionally a nitrogen protecting group when attached to a nitrogen atom;
R⁶ and R⁷ are each independently hydrogen, halogen, or optionally substituted C₁₋₆ alkyl; each X is independently halogen; and
n is 1, 2, 3, 4, or 5;
provided that the total collective carbon atom content of the alkyl, alkylene, alkenyl, alkenylene, alkynyl, and alkynylene groups of R^{A}, R^{M1}, and R^{M2}, excluding optional substituents, is from 10-40 carbon atoms, inclusive; and/or
provided that the total collective carbon atom content of the alkyl, alkylene, alkenyl, alkenylene, alkynyl, and alkynylene groups of R^{B} and R¹, excluding optional substituents, is from 10-40 carbon atoms, inclusive.

In certain embodiments, the compound of Formula (I) is of the formula:

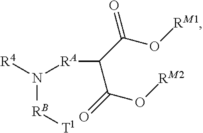

or a pharmaceutically acceptable salt thereof.

As defined herein, T¹ is selected from:

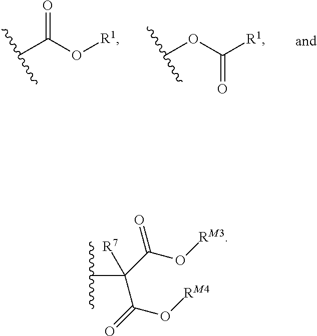

In certain embodiments, T¹ is:

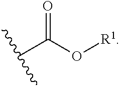

In certain embodiments, T¹ is:

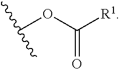

In certain embodiments, T¹ is:

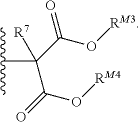

In certain embodiments, T¹ is:

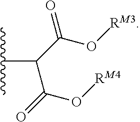

In certain embodiments, the compound of Formula (I) is of Formula (I-a):

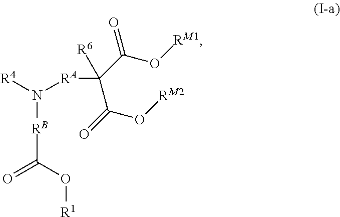

or a pharmaceutically acceptable salt thereof.

In certain embodiments, the compound of Formula (I-a) is of the formula:

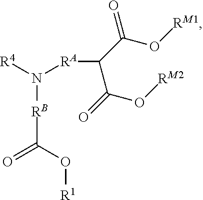

or a pharmaceutically acceptable salt thereof.

In certain embodiments, with respect to Formula (I) or (I-a), the total collective carbon atom content of the alkyl, alkylene, alkenyl, alkenylene, alkynyl, and alkynylene groups of R^{A}, R^{M1}, and R^{M2}, excluding optional substituents, is from 10-40 carbon atoms, inclusive.

In certain embodiments, with respect to Formula (I) or (I-a), the total collective carbon atom content of the alkyl, alkylene, alkenyl, alkenylene, alkynyl, and alkynylene groups of R^{A}, R^{M1}, and R^{M2}, excluding optional substituents, is from 10-30 carbon atoms, inclusive.

In certain embodiments, with respect to Formula (I) or (I-a), the total collective carbon atom content of the alkyl, alkylene, alkenyl, alkenylene, alkynyl, and alkynylene groups of R^{A}, R^{M1}, and R^{M2}, excluding optional substituents, is from 15-30 carbon atoms, inclusive.

In certain embodiments, with respect to Formula (I) or (I-a), the total collective carbon atom content of the alkyl, alkylene, alkenyl, alkenylene, alkynyl, and alkynylene groups of R^{A}, R^{M1}, and R^{M2}, excluding optional substituents, is from 15-25 carbon atoms, inclusive.

In certain embodiments, with respect to Formula (I) or (I-a), the total collective carbon atom content of the alkyl, alkylene, alkenyl, alkenylene, alkynyl, and alkynylene groups of R^{A}, R^{M1}, and R^{M2}, excluding optional substituents, is 10, 11, 12, 13, 14, 15, 16, 17, 18, 19, 20, 21, 22, 23, 24, 25, 26, 27, 28, 29, or 30 carbon atoms.

In certain embodiments, with respect to Formula (I) or (I-a), the total collective carbon atom content of the alkyl, alkylene, alkenyl, alkenylene, alkynyl, and alkynylene groups of R^{B} and R¹, excluding optional substituents, is from 10-40 carbon atoms, inclusive.

In certain embodiments, with respect to Formula (I) or (I-a), the total collective carbon atom content of the alkyl, alkylene, alkenyl, alkenylene, alkynyl, and alkynylene groups of R^{B} and R¹, excluding optional substituents, is from 15-40 carbon atoms, inclusive.

In certain embodiments, with respect to Formula (I) or (I-a), the total collective carbon atom content of the alkyl, alkylene, alkenyl, alkenylene, alkynyl, and alkynylene groups of R^{B} and R¹, excluding optional substituents, is from 15-30 carbon atoms, inclusive.

In certain embodiments, with respect to Formula (I) or (I-a), the total collective carbon atom content of the alkyl, alkylene, alkenyl, alkenylene, alkynyl, and alkynylene groups of R^{B} and R¹, excluding optional substituents, is from 20-30 carbon atoms, inclusive.

In certain embodiments, with respect to Formula (I) or (I-a), the total collective carbon atom content of the alkyl, alkylene, alkenyl, alkenylene, alkynyl, and alkynylene groups of R^{B} and R¹, excluding optional substituents, is from 15, 16, 17, 18, 19, 20, 21, 22, 23, 24, 25, 26, 27, 28, 29, 30, 31, 32, 33, 34, or 35 carbon atoms.

In certain embodiments, the compound of Formula (I) is of Formula (I-b):

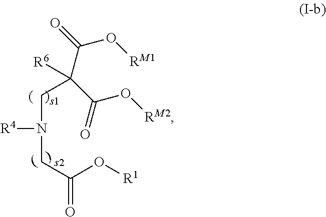

or a pharmaceutically acceptable salt thereof, wherein:
s1 and s2 are each independently an integer from 4-10, inclusive.

In certain embodiments, the compound of Formula (I-b) is of the formula:

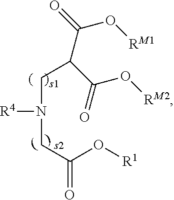

or a pharmaceutically acceptable salt thereof

In certain embodiments, the compound of Formula (I) is of Formula (I-c):

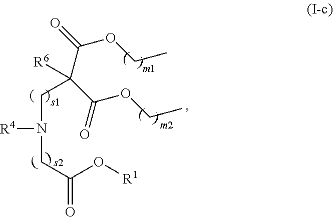

or a pharmaceutically acceptable salt thereof, wherein:
m1 and m2 are each independently 0 or an integer from 1-10, inclusive.

In certain embodiments, the compound of Formula (I-c) is of the formula:

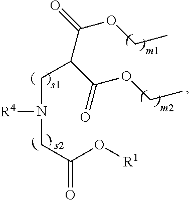

or a pharmaceutically acceptable salt thereof.

In certain embodiments, the compound of Formula (I) is of Formula (I-d):

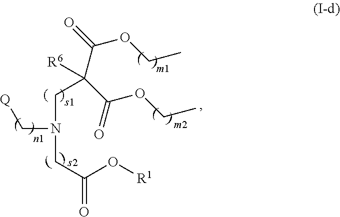

or a pharmaceutically acceptable salt thereof, wherein:
n1 is an integer from 1-10, inclusive.

In certain embodiments, the compound of Formula (I-d) is of the formula:

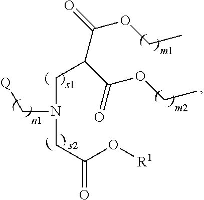

or a pharmaceutically acceptable salt thereof.

In certain embodiments, the compound of Formula (I) is of Formula (I-e):

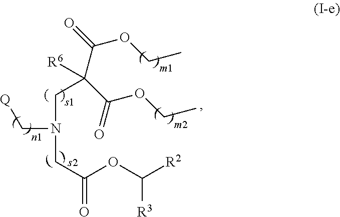

or a pharmaceutically acceptable salt thereof, wherein:

wherein R² and R³ are each independently optionally substituted, branched or unbranched C₁₋₂₀ alkyl, optionally substituted, branched or unbranched C₂₋₂₀ alkenyl, or optionally substituted, branched or unbranched C₂₋₂₀ alkynyl.

In certain embodiments, the compound of Formula (I-e) is of the formula:

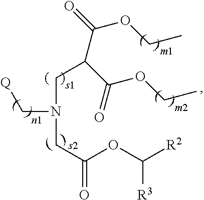

or a pharmaceutically acceptable salt thereof.

In certain embodiments, the compound of Formula (I) is of Formula (I-f):

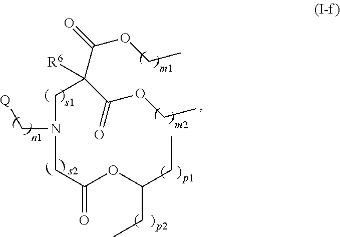

or a pharmaceutically acceptable salt thereof, wherein:
p1 and p2 are each independently 0 or an integer from 1-10, inclusive.

In certain embodiments, the compound of Formula (I-f) is of the formula:

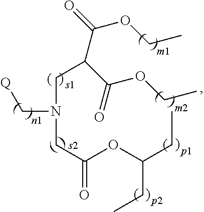

or a pharmaceutically acceptable salt thereof.

In certain embodiments, the compound of Formula (I) is selected from those in Table 1, and pharmaceutically acceptable salts thereof.

TABLE 1

| Compound | |
|---|---|
| No. | Structure |
| M1 | 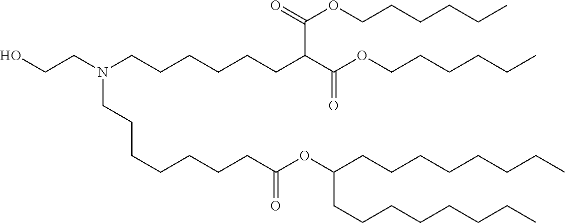 [SMILES: CCCCCCCCC(CCCCCCCC)OC(=O)CCCCCCCN(CCO)CCCCCCC(C(=O)OCCCCCC)C(=O)OCCCCCC] |
| M2 | 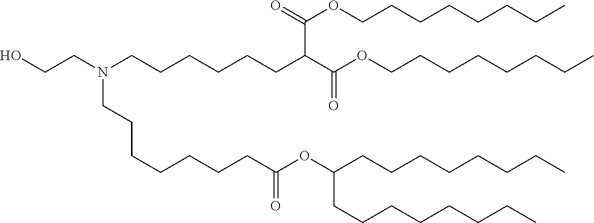 [SMILES: CCCCCCCCOC(=O)C(CCCCCCN(CCO)CCCCCCCC(=O)OC(CCCCCCCC)CCCCCCCC)C(=O)OCCCCCCCC] |
| M3 | 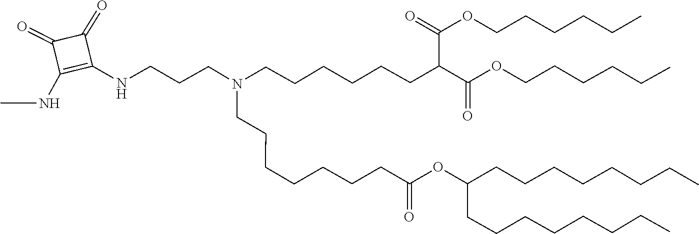 [SMILES: CCCCCCCCC(CCCCCCCC)OC(=O)CCCCCCCN(CCCCCCC(C(=O)OCCCCCC)C(=O)OCCCCCC)CCCNc1c(NC)c(=O)c1=O] |
| M4 | 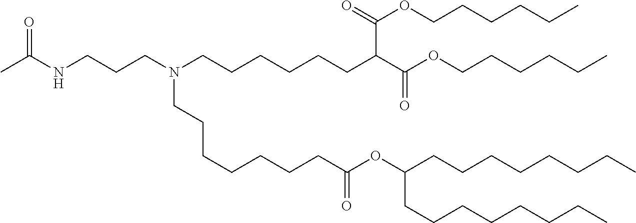 [SMILES: CCCCCCCCC(CCCCCCCC)OC(=O)CCCCCCCN(CCCCCCC(C(=O)OCCCCCC)C(=O)OCCCCCC)CCCNC(C)=O] |
| M5 | 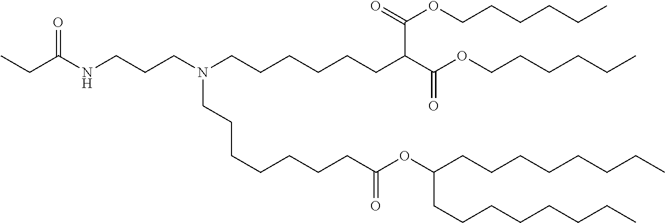 [SMILES: CCCCCCCCC(CCCCCCCC)OC(=O)CCCCCCCN(CCCCCCC(C(=O)OCCCCCC)C(=O)OCCCCCC)CCCNC(=O)CC] |
| M6 | 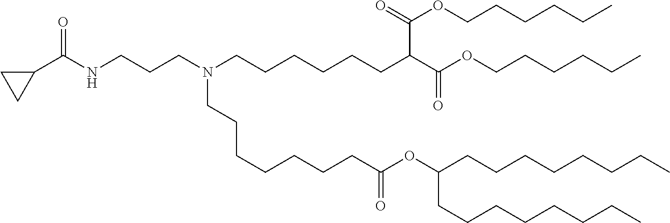 [SMILES: CCCCCCCCC(CCCCCCCC)OC(=O)CCCCCCCN(CCCCCCC(C(=O)OCCCCCC)C(=O)OCCCCCC)CCCNC(=O)C1CC1] |
| M7 | 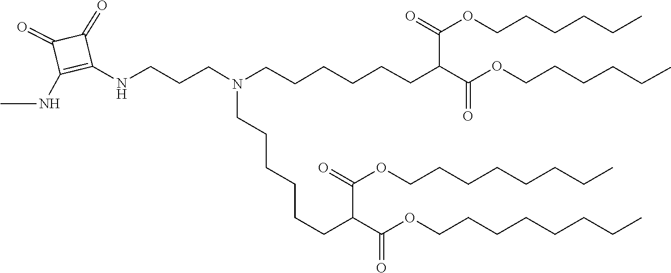 [SMILES: CCCCCCCCOC(=O)C(CCCCCCN(CCCCCCC(C(=O)OCCCCCC)C(=O)OCCCCCC)CCCNc1c(NC)c(=O)c1=O)C(=O)OCCCCCCCC] |
| M8 | 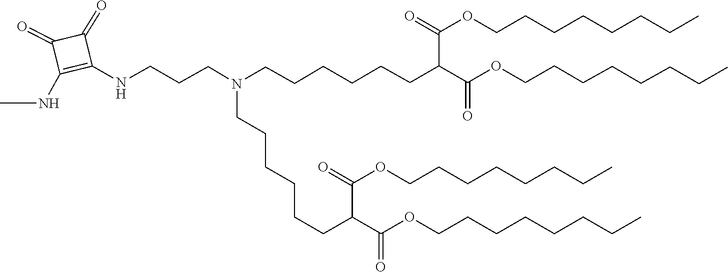 [SMILES: CCCCCCCCOC(=O)C(CCCCCCN(CCCCCCC(C(=O)OCCCCCCCC)C(=O)OCCCCCCCC)CCCNc1c(NC)c(=O)c1=O)C(=O)OCCCCCCCC] |
| M9 | 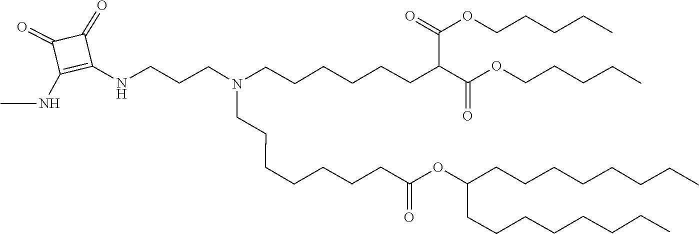 [SMILES: CCCCCCCCC(CCCCCCCC)OC(=O)CCCCCCCN(CCCCCCC(C(=O)OCCCCC)C(=O)OCCCCC)CCCNc1c(NC)c(=O)c1=O] |
| M10 | 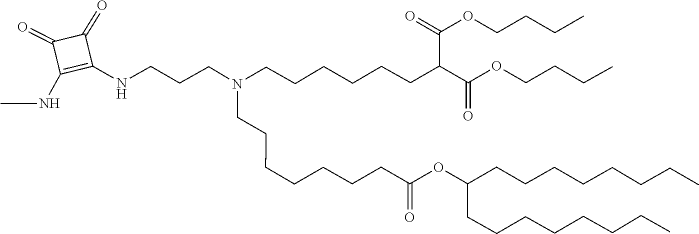 [SMILES: CCCCCCCCC(CCCCCCCC)OC(=O)CCCCCCCN(CCCCCCC(C(=O)OCCCC)C(=O)OCCCC)CCCNc1c(NC)c(=O)c1=O] |
| M11 | 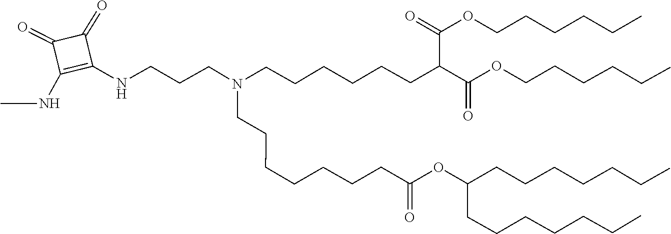 [SMILES: CCCCCCCC(CCCCCCC)OC(=O)CCCCCCCN(CCCCCCC(C(=O)OCCCCCC)C(=O)OCCCCCC)CCCNc1c(NC)c(=O)c1=O] |
| M12 | 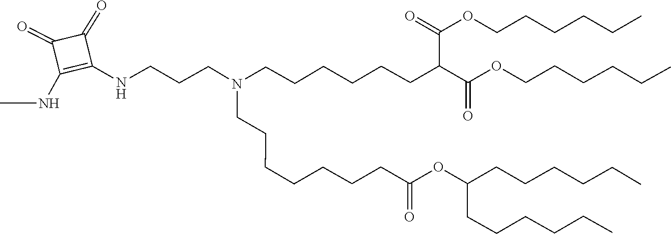 [SMILES: CCCCCCOC(=O)C(CCCCCCN(CCCCCCCC(=O)OC(CCCCCC)CCCCCC)CCCNc1c(NC)c(=O)c1=O)C(=O)OCCCCCC] |
| M13 | 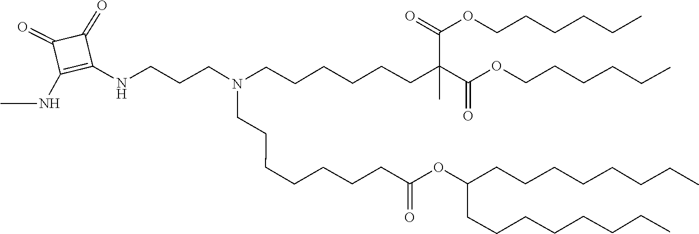 [SMILES: CCCCCCCCC(CCCCCCCC)OC(=O)CCCCCCCN(CCCCCCC(C)(C(=O)OCCCCCC)C(=O)OCCCCCC)CCCNc1c(NC)c(=O)c1=O] |
| M14 | 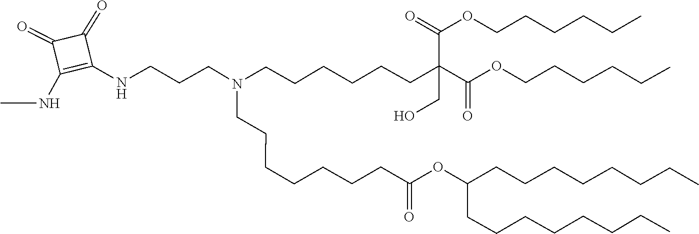 [SMILES: CCCCCCCCC(CCCCCCCC)OC(=O)CCCCCCCN(CCCCCCC(CO)(C(=O)OCCCCCC)C(=O)OCCCCCC)CCCNc1c(NC)c(=O)c1=O] |
| M15 | 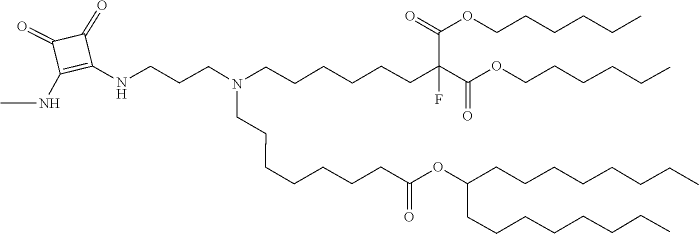 [SMILES: CCCCCCCCC(CCCCCCCC)OC(=O)CCCCCCCN(CCCCCCC(F)(C(=O)OCCCCCC)C(=O)OCCCCCC)CCCNc1c(NC)c(=O)c1=O] |
| M16 | 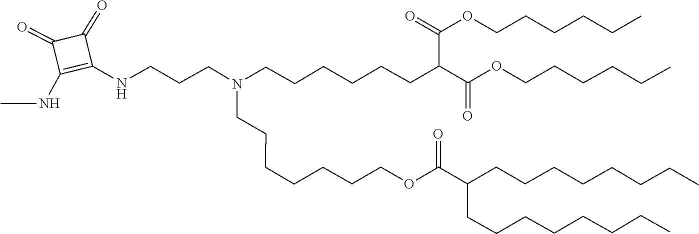 [SMILES: CCCCCCCCC(CCCCCCCC)C(=O)OCCCCCCCN(CCCCCCC(C(=O)OCCCCCC)C(=O)OCCCCCC)CCCNc1c(NC)c(=O)c1=O] |
| M17 | 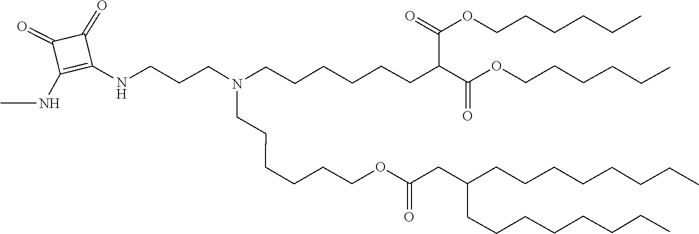 [SMILES: CCCCCCCCC(CCCCCCCC)CC(=O)OCCCCCCN(CCCCCCC(C(=O)OCCCCCC)C(=O)OCCCCCC)CCCNc1c(NC)c(=O)c1=O] |

In the various aspects and embodiments disclosed herein, express reference to a compound of Formula (I) is understood to alternatively refer to a compound of any disclosed subgenus (e.g., Formula (I-a), (I-b), (I-c), (I-d), (I-e), (I-f)) or species thereof, for example, to a compound of Table 1. The following definitions and embodiments apply to all generic formulae comprising the relevant groups (e.g., Formula (I) or any subgeneric structure thereof) provided herein.

R^{A} and R^{B}

In certain embodiments, R^{A} is optionally substituted C₁₋₂₀ alkylene, optionally substituted C₁₋₂₀ alkenylene, or optionally substituted C₁₋₂₀ alkynylene. In certain embodiments, R^{A} is optionally substituted C₁₋₂₀ alkylene. In certain embodiments, R^{A} is unsubstituted C₁₋₂₀ alkylene. In certain embodiments, R^{A} is optionally substituted C₁₋₂₀ alkenylene. In certain embodiments, R^{A} is optionally substituted C₁₋₂₀ alkynylene.

In certain embodiments, R^{A} is optionally substituted C₄₋₂₀ alkylene, optionally substituted C₄₋₂₀ alkenylene, or optionally substituted C₄₋₂₀ alkynylene. In certain embodiments, R^{A} is optionally substituted C₄₋₂₀ alkylene. In certain embodiments, R^{A} is unsubstituted C₄₋₂₀ alkylene. In certain embodiments, R^{A} is optionally substituted C₄₋₂₀ alkenylene. In certain embodiments, R^{A} is optionally substituted C₄₋₂₀ alkynylene.

In certain embodiments, R^{A} is optionally substituted C₄₋₁₀ alkylene. In certain embodiments, R^{A} is optionally substituted C₄ alkylene. In certain embodiments, R^{A} is optionally substituted C₅ alkylene. In certain embodiments, R^{A} is optionally substituted C₆ alkylene. In certain embodiments, R^{A} is optionally substituted C₇ alkylene. In certain embodiments, R^{A} is optionally substituted C₈ alkylene. In certain embodiments, R^{A} is optionally substituted C₉ alkylene. In certain embodiments, R^{A} is optionally substituted C₁₀ alkylene.

In certain embodiments, R^{A} is unsubstituted C₄₋₁₀ alkylene. In certain embodiments, R^{A} is:

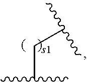

wherein s1 is as defined herein. In certain embodiments, R^{A} is selected from:

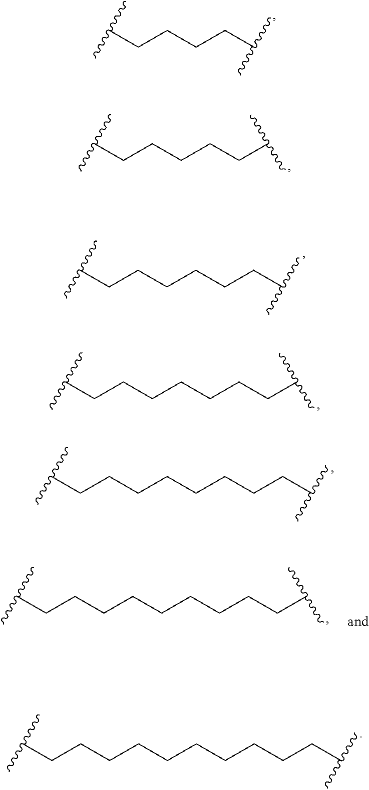

In certain embodiments, R^{A} is:

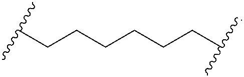 [SMILES: *CCCCCC*]

In certain embodiments, R^{B} is optionally substituted C₁₋₂₀ alkylene, optionally substituted C₁₋₂₀ alkenylene, or optionally substituted C₁₋₂₀ alkynylene. In certain embodiments, R^{B} is optionally substituted C₁₋₂₀ alkylene. In certain embodiments, R^{B} is unsubstituted C₁₋₂₀ alkylene. In certain embodiments, R^{B} is optionally substituted C₁₋₂₀ alkenylene. In certain embodiments, R^{B} is optionally substituted C₁₋₂₀ alkynylene.

In certain embodiments, R^{B} is optionally substituted C₄₋₂₀ alkylene, optionally substituted C₄₋₂₀ alkenylene, or optionally substituted C₄₋₂₀ alkynylene. In certain embodiments, R^{B} is optionally substituted C₄₋₂₀ alkylene. In certain embodiments, R^{B} is unsubstituted C₄₋₂₀ alkylene. In certain embodiments, R^{B} is optionally substituted C₄₋₂₀ alkenylene. In certain embodiments, R^{B} is optionally substituted C₄₋₂₀ alkynylene.

In certain embodiments, R^{B} is optionally substituted C₄₋₁₀ alkylene. In certain embodiments, R^{B} is optionally substituted C₄ alkylene. In certain embodiments, R^{B} is optionally substituted C₅ alkylene. In certain embodiments, R^{B} is optionally substituted C₆ alkylene. In certain embodiments, R^{B} is optionally substituted C₇ alkylene. In certain embodiments, R^{B} is optionally substituted C₈ alkylene. In certain embodiments, R^{B} is optionally substituted C₉ alkylene. In certain embodiments, R^{B} is optionally substituted C₁₀ alkylene.

In certain embodiments, R^{B} is unsubstituted C₄₋₁₀ alkylene. In certain embodiments, R^{B} is:

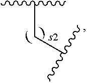

wherein s2 is as defined herein. In certain embodiments, R^{B} is selected from:

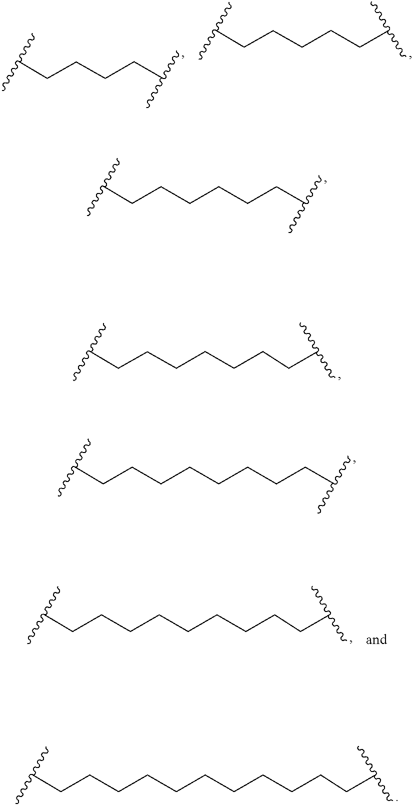

In certain embodiments, R^{B} is:

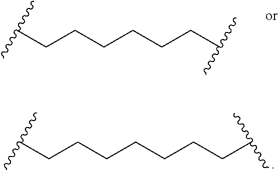 [SMILES: *CCCCCC* ; *CCCCCCC*]

R^{M1}, R^{M2}, R^{M3}, and R^{M4}

As defined herein, R^{M1} is optionally substituted, branched or unbranched C₁₋₂₀ alkyl, optionally substituted, branched or unbranched C₂₋₂₀ alkenyl, or optionally substituted, branched or unbranched C₂₋₂₀ alkynyl. In certain embodiments, R^{M1} is optionally substituted, branched or unbranched C₁₋₂₀ alkyl. In certain embodiments, R^{M1} is unsubstituted, branched or unbranched C₁₋₂₀ alkyl. In certain embodiments, R^{M1} is unsubstituted, unbranched C₁₋₂₀ alkyl. In certain embodiments, R^{M1} is optionally substituted, branched or unbranched C₂₋₂₀ alkenyl. In certain embodiments, R^{M1} is optionally substituted, branched or unbranched C₂₋₂₀ alkynyl.

In certain embodiments, R^{M1} is optionally substituted, branched or unbranched C₁₋₁₀ alkyl. In certain embodiments, R^{M1} is optionally substituted C₁ alkyl. In certain embodiments, R^{M1} is optionally substituted C₂ alkyl. In certain embodiments, R^{M1} is optionally substituted, branched or unbranched C₃ alkyl. In certain embodiments, R^{M1} is optionally substituted, branched or unbranched C₄ alkyl. In certain embodiments, R^{M1} is optionally substituted, branched or unbranched C₅ alkyl. In certain embodiments, R^{M1} is optionally substituted, branched or unbranched C₆ alkyl. In certain embodiments, R^{M1} is optionally substituted, branched or unbranched C₇ alkyl. In certain embodiments, R^{M1} is optionally substituted, branched or unbranched C₈ alkyl. In certain embodiments, R^{M1} is optionally substituted, branched or unbranched C₉ alkyl. In certain embodiments, R^{M1} is optionally substituted, branched or unbranched C₁₀ alkyl.

In certain embodiments, R^{M1} is unsubstituted, branched or unbranched C₁₋₁₀ alkyl. In certain embodiments, R^{M1} is unsubstituted, unbranched C₁₋₁₀ alkyl. In certain embodiments, R^{M1} is of the formula:

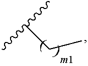

wherein m1 is as defined herein. In certain embodiments, R^{M1} is:

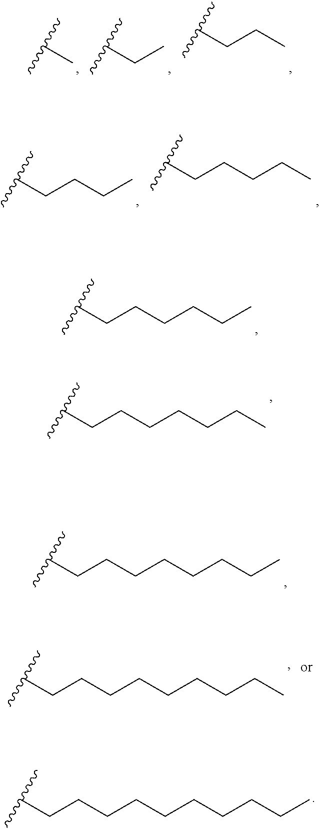

In certain embodiments, R^{M1} is:

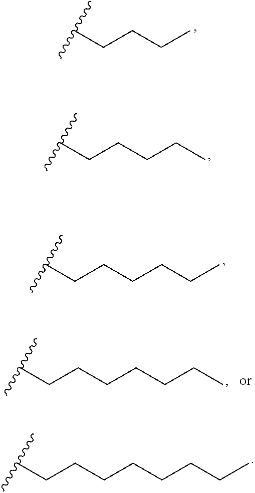

As defined herein, R^{M2} is optionally substituted, branched or unbranched C₁₋₂₀ alkyl, optionally substituted, branched or unbranched C₂₋₂₀ alkenyl, or optionally substituted, branched or unbranched C₂₋₂₀ alkynyl. In certain embodiments, R^{M2} is optionally substituted, branched or unbranched C₁₋₂₀ alkyl. In certain embodiments, R^{M2} is unsubstituted, branched or unbranched C₁₋₂₀ alkyl. In certain embodiments, R^{M2} is unsubstituted, unbranched C₁₋₂₀ alkyl. In certain embodiments, R^{M2} is optionally substituted, branched or unbranched C₂₋₂₀ alkenyl. In certain embodiments, R^{M2} is optionally substituted, branched or unbranched C₂₋₂₀ alkynyl.

In certain embodiments, R^{M2} is optionally substituted, branched or unbranched C₁₋₁₀ alkyl. In certain embodiments, R^{M2} is optionally substituted C₁ alkyl. In certain embodiments, R^{M2} is optionally substituted C₂ alkyl. In certain embodiments, R^{M2} is optionally substituted, branched or unbranched C₃ alkyl. In certain embodiments, R^{M2} is optionally substituted, branched or unbranched C₄ alkyl. In certain embodiments, R^{M2} is optionally substituted, branched or unbranched C₅ alkyl. In certain embodiments, R^{M2} is optionally substituted, branched or unbranched C₆ alkyl. In certain embodiments, R^{M2} is optionally substituted, branched or unbranched C₇ alkyl. In certain embodiments, R^{M2} is optionally substituted, branched or unbranched C₈ alkyl. In certain embodiments, R^{M2} is optionally substituted, branched or unbranched C₉ alkyl. In certain embodiments, R^{M2} is optionally substituted, branched or unbranched C₁₀ alkyl.

In certain embodiments, R^{M2} is unsubstituted, branched or unbranched C₁₋₁₀ alkyl. In certain embodiments, R^{M2} is unsubstituted, unbranched C₁₋₁₀ alkyl. In certain embodiments, R^{M2} is of the formula:

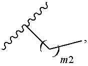

wherein m2 is as defined herein. In certain embodiments, R^{M2} is:

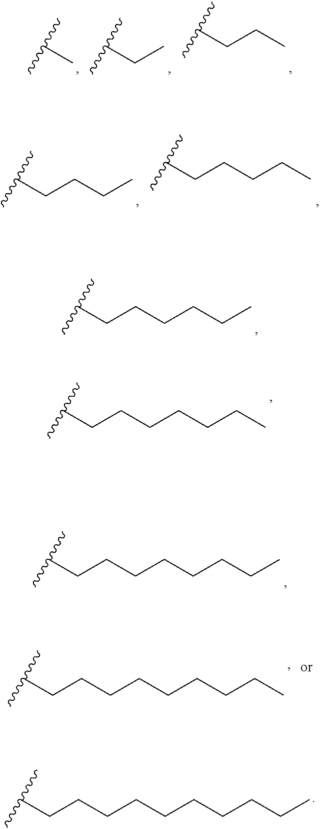

In certain embodiments, R^{M2} is:

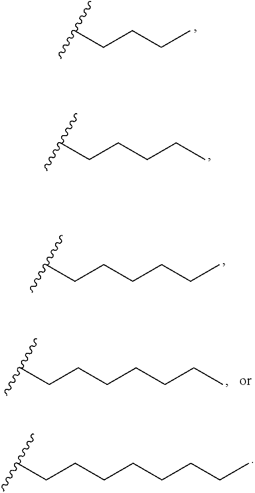

As defined herein, R^{M3} is optionally substituted, branched or unbranched C₁₋₂₀ alkyl, optionally substituted, branched or unbranched C₂₋₂₀ alkenyl, or optionally substituted, branched or unbranched C₂₋₂₀ alkynyl. In certain embodiments, R^{M3} is optionally substituted, branched or unbranched C₁₋₂₀ alkyl. In certain embodiments, R^{M3} is unsubstituted, branched or unbranched C₁₋₂₀ alkyl. In certain embodiments, R^{M3} is unsubstituted, unbranched C₁₋₂₀ alkyl. In certain embodiments, R^{M3} is optionally substituted, branched or unbranched C₂₋₂₀ alkenyl. In certain embodiments, R^{M3} is optionally substituted, branched or unbranched C₂₋₂₀ alkynyl.

In certain embodiments, R^{M3} is optionally substituted, branched or unbranched C₁₋₁₀ alkyl. In certain embodiments, R^{M3} is optionally substituted C₁ alkyl. In certain embodiments, R^{M3} is optionally substituted C₂ alkyl. In certain embodiments, R^{M3} is optionally substituted, branched or unbranched C₃ alkyl. In certain embodiments, R^{M3} is optionally substituted, branched or unbranched C₄ alkyl. In certain embodiments, R^{M3} is optionally substituted, branched or unbranched C₅ alkyl. In certain embodiments, R^{M3} is optionally substituted, branched or unbranched C₆ alkyl. In certain embodiments, R^{M3} is optionally substituted, branched or unbranched C₇ alkyl. In certain embodiments, R^{M3} is optionally substituted, branched or unbranched C₈ alkyl. In certain embodiments, R^{M3} is optionally substituted, branched or unbranched C₉ alkyl. In certain embodiments, R^{M3} is optionally substituted, branched or unbranched C₁₀ alkyl.

In certain embodiments, R^{M3} is unsubstituted, branched or unbranched C₁₋₁₀ alkyl. In certain embodiments, R^{M3} is unsubstituted, unbranched C₁₋₁₀ alkyl. In certain embodiments, R^{M3} is:

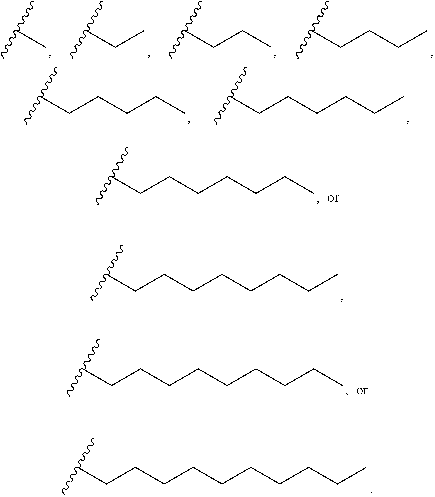

In certain embodiments, R^{M3} is:

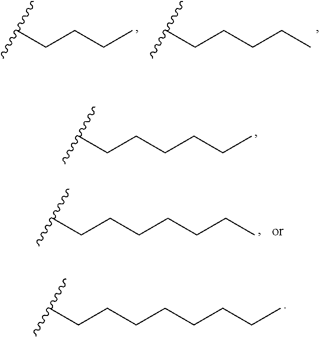

As defined herein, R^{M4} is optionally substituted, branched or unbranched C₁₋₂₀ alkyl, optionally substituted, branched or unbranched C₂₋₂₀ alkenyl, or optionally substituted, branched or unbranched C₂₋₂₀ alkynyl. In certain embodiments, R^{M4} is optionally substituted, branched or unbranched C₁₋₂₀ alkyl. In certain embodiments, R^{M4} is unsubstituted, branched or unbranched C₁₋₂₀ alkyl. In certain embodiments, R^{M4} is unsubstituted, unbranched C₁₋₂₀ alkyl. In certain embodiments, R^{M4} is optionally substituted, branched or unbranched C₂₋₂₀ alkenyl. In certain embodiments, R^{M4} is optionally substituted, branched or unbranched C₂₋₂₀ alkynyl.

In certain embodiments, R^{M4} is optionally substituted, branched or unbranched C₁₋₁₀ alkyl. In certain embodiments, R^{M4} is optionally substituted C₁ alkyl. In certain embodiments, R^{M4} is optionally substituted C₂ alkyl. In certain embodiments, R^{M4} is optionally substituted, branched or unbranched C₃ alkyl. In certain embodiments, R^{M4} is optionally substituted, branched or unbranched C₄ alkyl. In certain embodiments, R^{M4} is optionally substituted, branched or unbranched C₅ alkyl. In certain embodiments, R^{M4} is optionally substituted, branched or unbranched C₆ alkyl. In certain embodiments, R^{M4} is optionally substituted, branched or unbranched C₇ alkyl. In certain embodiments, R^{M4} is optionally substituted, branched or unbranched C₈ alkyl. In certain embodiments, R^{M4} is optionally substituted, branched or unbranched C₉ alkyl. In certain embodiments, R^{M4} is optionally substituted, branched or unbranched C₁₀ alkyl.

In certain embodiments, R^{M4} is unsubstituted, branched or unbranched C₁₋₁₀ alkyl. In certain embodiments, R^{M4} is unsubstituted, unbranched C₁₋₁₀ alkyl. In certain embodiments, R^{M4} is:

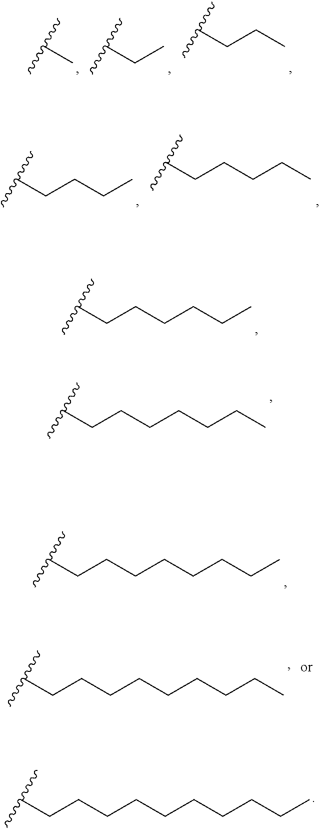

In certain embodiments, R^{M4} is:

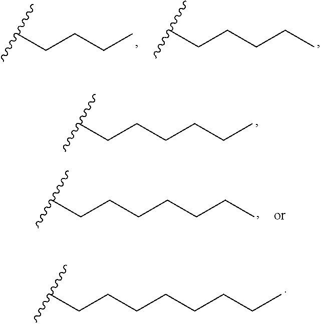

R¹, R², and R³

As defined herein, R¹ is optionally substituted, branched or unbranched C₁₋₃₀ alkyl, optionally substituted, branched or unbranched C₂₋₃₀ alkenyl, or optionally substituted, branched or unbranched C₂₋₃₀ alkynyl. In certain embodiments, R¹ is optionally substituted, branched or unbranched C₁₋₃₀ alkyl. In certain embodiments, R¹ is optionally substituted, branched C₃₋₃₀ alkyl. In certain embodiments, R¹ is unsubstituted, branched or unbranched C₁₋₃₀ alkyl. In certain embodiments, R¹ is unsubstituted, branched C₃₋₃₀ alkyl. In certain embodiments, R¹ is optionally substituted, branched or unbranched C₂₋₃₀ alkenyl. In certain embodiments, R¹ is optionally substituted, branched or unbranched C₂₋₃₀ alkynyl.

In certain embodiments, R¹ is optionally substituted, branched or unbranched C₁₀₋₃₀ alkyl. In certain embodiments, R¹ is optionally substituted, branched C₁₀₋₃₀ alkyl. In certain embodiments, R¹ is unsubstituted, branched or unbranched C₁₀₋₃₀ alkyl. In certain embodiments, R¹ is unsubstituted, branched C₁₀₋₃₀ alkyl.

In certain embodiments, R¹ is optionally substituted, branched or unbranched C₁₀₋₂₀ alkyl. In certain embodiments, R¹ is optionally substituted, branched C₁₀₋₂₀ alkyl. In certain embodiments, R¹ is unsubstituted, branched or unbranched C₁₀₋₂₀ alkyl. In certain embodiments, R¹ is unsubstituted, branched C₁₀₋₂₀ alkyl.

In certain embodiments, R¹ is optionally substituted, branched or unbranched C₁₋₂₀ alkyl. In certain embodiments, R¹ is optionally substituted, branched C₃₋₂₀ alkyl. In certain embodiments, R¹ is unsubstituted, branched or unbranched C₁₋₂₀ alkyl. In certain embodiments, R¹ is unsubstituted, branched C₃₋₂₀ alkyl.

In certain embodiments, R¹ is selected from:

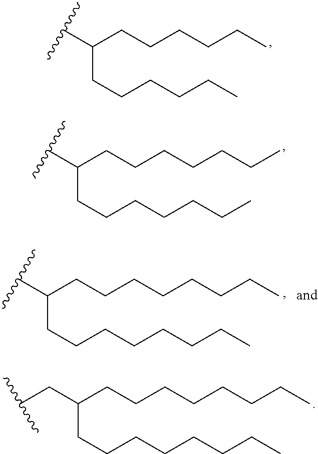 [SMILES: *C(CCCCCC)CCCCCC ; *C(CCCCCCC)CCCCCCC ; *C(CCCCCCCC)CCCCCCCC ; *CC(CCCCCCCC)CCCCCCCC]

In certain embodiments, R¹ is of the formula:

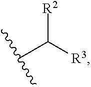

wherein R² and R³ are as defined herein. In certain embodiments, R¹ is of the formula:

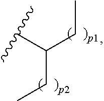

wherein p1 and p2 are as defined herein.

As defined herein, R² is optionally substituted, branched or unbranched C₁₋₂₀ alkyl, optionally substituted, branched or unbranched C₂₋₂₀ alkenyl, or optionally substituted, branched or unbranched C₂₋₂₀ alkynyl. In certain embodiments, R² is optionally substituted, branched or unbranched C₁₋₂₀ alkyl. In certain embodiments, R² is unsubstituted, branched or unbranched C₁₋₂₀ alkyl. In certain embodiments, R² is unsubstituted, unbranched C₁₋₂₀ alkyl. In certain embodiments, R² is optionally substituted C₂₋₂₀ alkenyl. In certain embodiments, R² is optionally substituted C₂₋₂₀ alkynyl.

In certain embodiments, R² is optionally substituted, branched or unbranched C₁₋₁₀ alkyl. In certain embodiments, R² is optionally substituted C₁ alkyl. In certain embodiments, R² is optionally substituted C₂ alkyl. In certain embodiments, R² is optionally substituted, branched or unbranched C₃ alkyl. In certain embodiments, R² is optionally substituted, branched or unbranched C₄ alkyl. In certain embodiments, R² is optionally substituted, branched or unbranched C₅ alkyl. In certain embodiments, R² is optionally substituted, branched or unbranched C₆ alkyl. In certain embodiments, R² is optionally substituted, branched or unbranched C₇ alkyl. In certain embodiments, R² is optionally substituted, branched or unbranched C₈ alkyl. In certain embodiments, R² is optionally substituted, branched or unbranched C₉ alkyl. In certain embodiments, R² is optionally substituted, branched or unbranched C₁₀ alkyl.

In certain embodiments, R² is unsubstituted, branched or unbranched C₁₋₁₀ alkyl. In certain embodiments, R² is unsubstituted, unbranched C₁₋₁₀ alkyl. In certain embodiments, R² is of the formula:

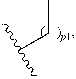

wherein p1 is as defined herein. In certain embodiments, R² is:

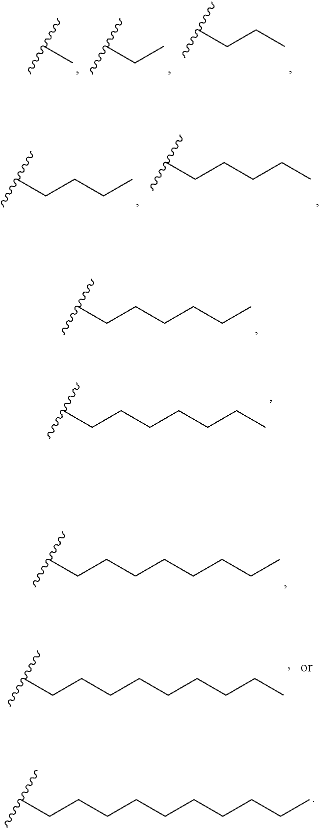

In certain embodiments, R² is:

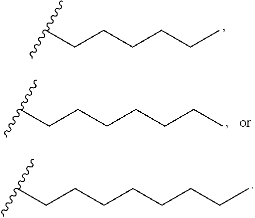

As defined herein, R³ is optionally substituted, branched or unbranched C₁₋₂₀ alkyl, optionally substituted, branched or unbranched C₂₋₂₀ alkenyl, or optionally substituted, branched or unbranched C₂₋₂₀ alkynyl. In certain embodiments, R³ is optionally substituted, branched or unbranched C₁₋₂₀ alkyl. In certain embodiments, R³ is unsubstituted, branched or unbranched C₁₋₂₀ alkyl. In certain embodiments, R³ is unsubstituted, unbranched C₁₋₂₀ alkyl. In certain embodiments, R³ is optionally substituted, branched or unbranched C₂₋₂₀ alkenyl. In certain embodiments, R³ is optionally substituted, branched or unbranched C₂₋₂₀ alkynyl.

In certain embodiments, R³ is optionally substituted, branched or unbranched C₁₋₁₀ alkyl. In certain embodiments, R³ is optionally substituted C₁ alkyl. In certain embodiments, R³ is optionally substituted C₂ alkyl. In certain embodiments, R³ is optionally substituted, branched or unbranched C₃ alkyl. In certain embodiments, R³ is optionally substituted, branched or unbranched C₄ alkyl. In certain embodiments, R³ is optionally substituted, branched or unbranched C₅ alkyl. In certain embodiments, R³ is optionally substituted, branched or unbranched C₆ alkyl. In certain embodiments, R³ is optionally substituted, branched or unbranched C₇ alkyl. In certain embodiments, R³ is optionally substituted, branched or unbranched C₈ alkyl. In certain embodiments, R³ is optionally substituted, branched or unbranched C₉ alkyl. In certain embodiments, R³ is optionally substituted, branched or unbranched C₁₀ alkyl.

In certain embodiments, R³ is unsubstituted, branched or unbranched C₁₋₁₀ alkyl. In certain embodiments, R³ is unsubstituted, unbranched C₁₋₁₀ alkyl. In certain embodiments, R³ is as defined herein:

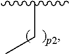

wherein p2 is as defined herein. In certain embodiments, R³ is:

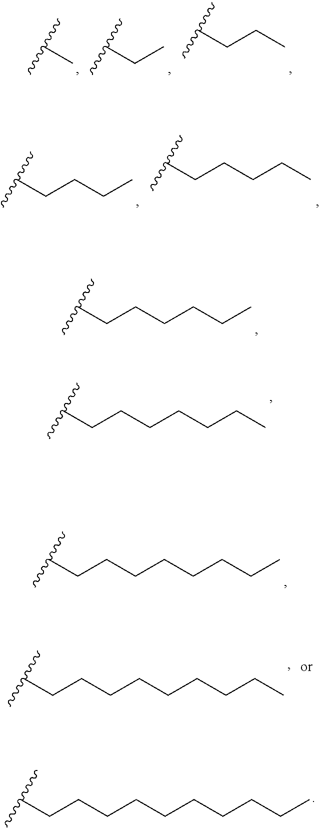

In certain embodiments, R³ is:

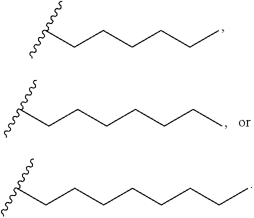

R⁴, Y¹, Q, and R⁸

As defined herein, R⁴ is a head group selected from optionally substituted C₁₋₆ alkyl, optionally substituted C₂₋₆ alkenyl, optionally substituted C₂₋₆ alkynyl, optionally substituted C₃₋₈ carbocyclyl, and —Y¹-Q. In certain embodiments, R⁴ is optionally substituted C₁₋₆ alkyl. In certain embodiments, R⁴ is optionally substituted C₂₋₆ alkenyl. In certain embodiments, R⁴ is optionally substituted C₂₋₆ alkynyl. In certain embodiments, R⁴ is optionally substituted C₃₋₈ carbocyclyl. In certain embodiments, R⁴ is —Y¹-Q. In certain embodiments, R⁴ is of the formula:

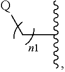

wherein n1 is as defined herein.

As defined herein, Y¹ is optionally substituted C₁₋₁₀ alkylene, optionally substituted C₂₋₁₀ alkenylene, and optionally substituted C₂₋₁₀ alkynylene. In certain embodiments, Y¹ is optionally substituted C₁₋₁₀ alkylene. In certain embodiments, Y¹ is unsubstituted C₁₋₁₀ alkylene. In certain embodiments, Y¹ is optionally substituted C₂₋₁₀ alkenylene. In certain embodiments, Y¹ is and optionally substituted C₂₋₁₀ alkynylene.

In certain embodiments, Y¹ is optionally substituted C₁₋₄ alkylene. In certain embodiments, Y¹ is optionally substituted C₁ alkylene. In certain embodiments, Y¹ is optionally substituted C₂ alkylene. In certain embodiments, Y¹ is optionally substituted C₃ alkylene. In certain embodiments, Y¹ is optionally substituted C₄ alkylene.

In certain embodiments, Y¹ is unsubstituted C₁₋₄ alkylene. In certain embodiments, Y¹ is of the formula:

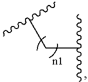

wherein n1 is defined herein. In certain embodiments, wherein Y¹ is:

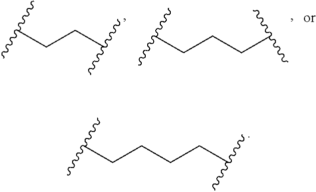

As defined herein, Q is optionally substituted C₃₋₈ carbocyclyl, optionally substituted C₆₋₁₀ aryl, optionally substituted C₃₋₈ heterocyclyl, optionally substituted C₅₋₁₀ heteroaryl, —OR^{O}, —O(CH₂)ₙN(R^{N})₂, —O(CH₂)ₙOR^{O}, —C(O)OR^{O}, —OC(O)R, —OC(O)OR^{O}, —CX₃, —CX₂H, —CXH₂, —CN, —N(R^{N})₂, —N(R^{N})(CH₂)ₙN(R^{N})₂, —N(R^{N})(CH₂)ₙOR^{O}, —C(O)N(R^{N})₂, —N(R^{N})C(O)R, —N(R^{N})C(O)N(R^{N})₂, —N(R^{N})C(S)N(R^{N})₂, —N(R^{N})C(═NR⁵)N(R^{N})₂, —N(R^{N})C(═CHR⁵)N(R^{N})₂, —C(═NR⁵)N(R^{N})₂, —C(═NR⁵)R, —OC(O)N(R^{N})₂, —N(R^{N})C(O)OR^{O}, —C(R)N(R^{N})₂C(O)OR^{O}, —N(R^{N})S(O)₂R, —N(R^{N})S(O)R, —S(O)₂N(R^{N})₂, —S(O)N(R^{N})₂,

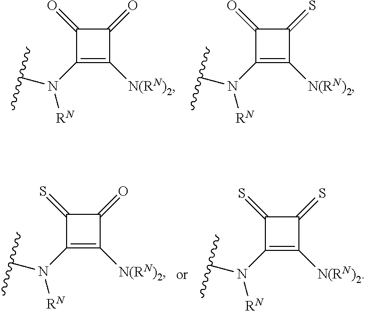

In certain embodiments, Q is —N(R^{N})R⁸, wherein R⁸ is —C(O)R, —C(O)N(R^{N})₂, —C(S)N(R^{N})₂, —C(═NR⁵)N(R^{N})₂, —C(═CHR⁵)N(R^{N})₂, —C(O)OR^{O}, —S(O)₂R, —S(O)R,

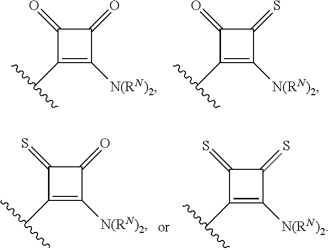

In certain embodiments, Q is —NHR⁸.

In certain embodiments, R⁸ is —C(O)R. In certain embodiments, R⁸ is —C(O)Me, —C(O)Et, or

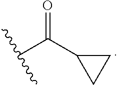 [SMILES: *C(=O)C1CC1]

In certain embodiments, R⁸ is of the formula:

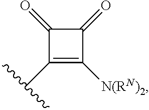

wherein R^{N} is as defined herein. In certain embodiments, R⁸ is:

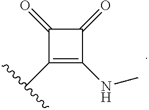 [SMILES: *c1c(NC)c(=O)c1=O]

In certain embodiments, Q is —OR^{O}. In certain embodiments, Q is —OH.

In certain embodiments, Q is —N(R^{N})C(O)R. In certain embodiments, Q is —NHC(O)Me, —NHC(O)Et, or

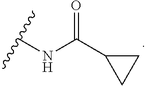 [SMILES: *NC(=O)C1CC1]

In certain embodiments, Q is of the formula:

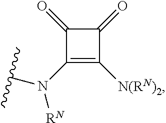

wherein R^{N} is as defined herein. In certain embodiments, Q is:

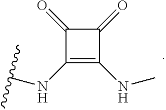 [SMILES: *Nc1c(NC)c(=O)c1=O]

In certain embodiments, R⁴ is a head group selected from:

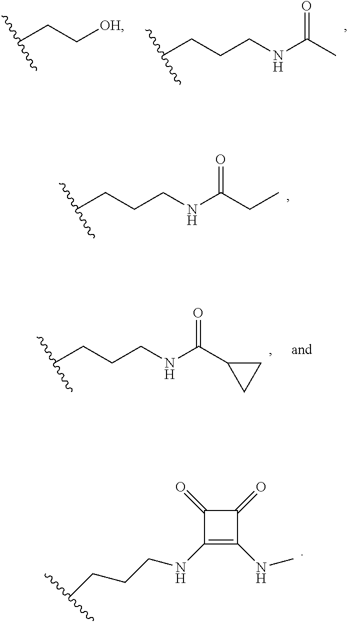

In certain embodiments, R⁴ is a head group selected from:

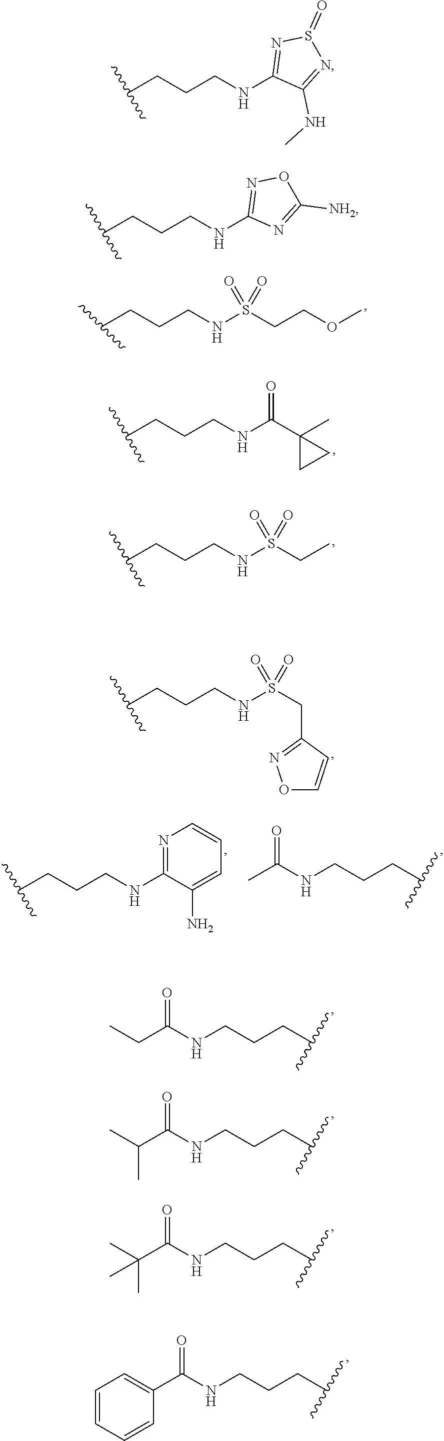

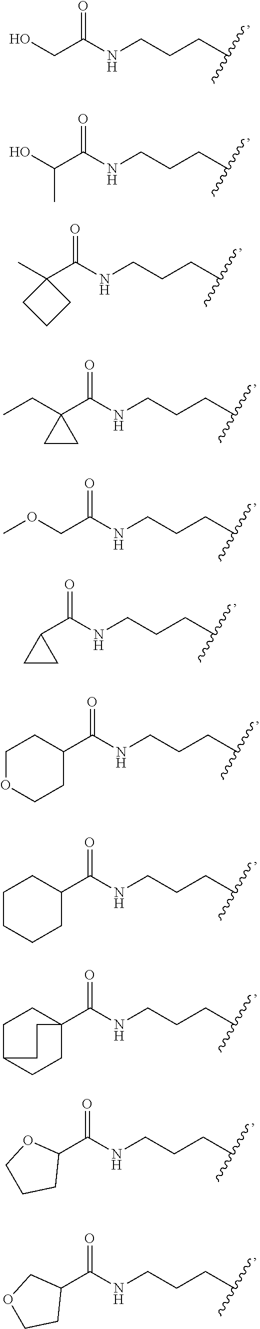

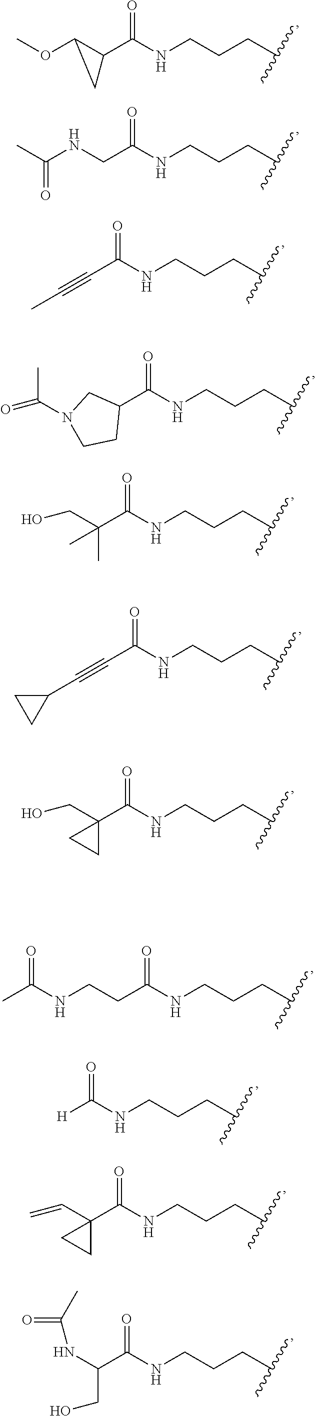

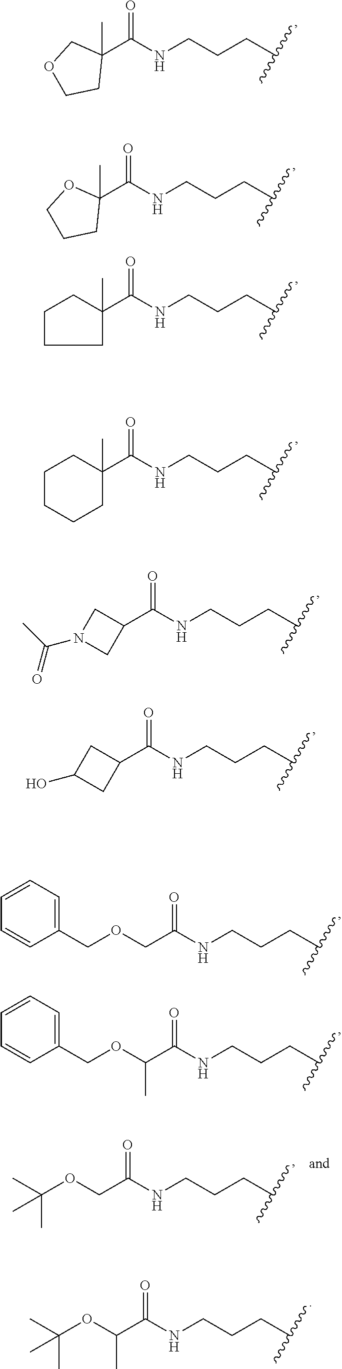

In certain embodiments, R⁴ is a head group selected from:

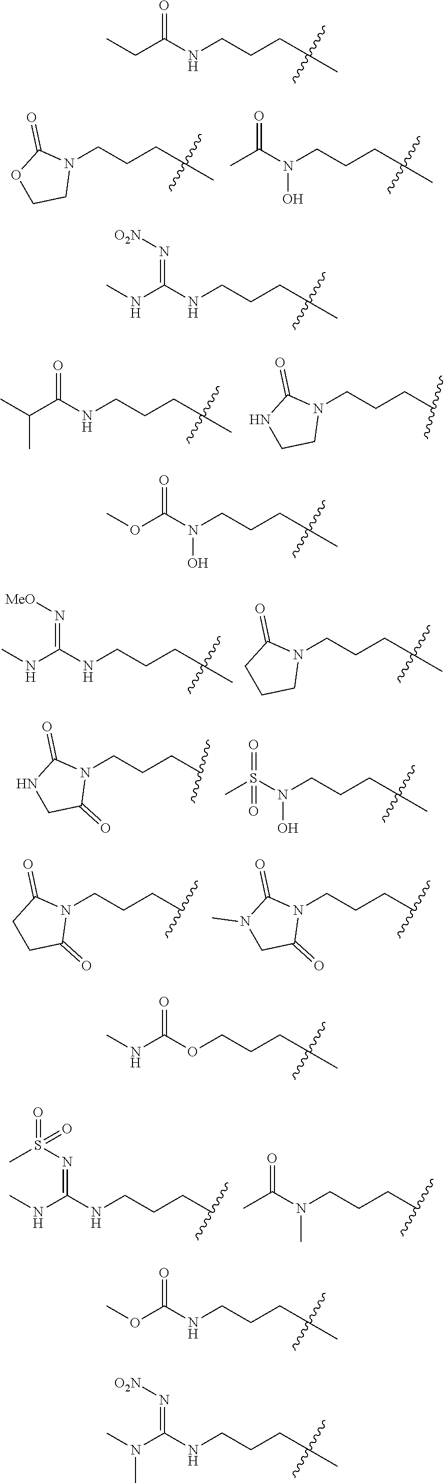

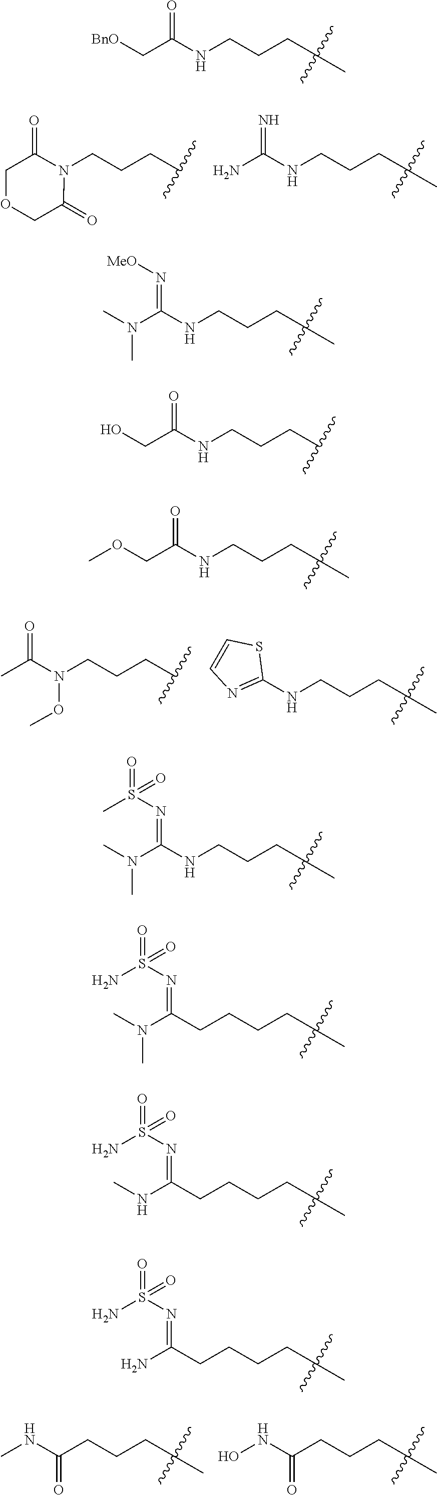

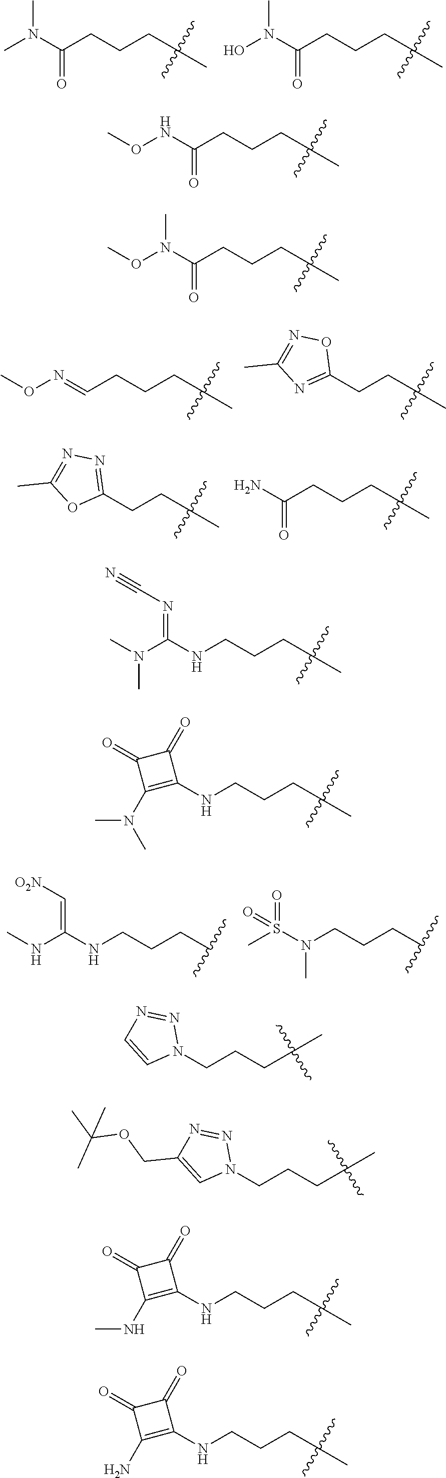

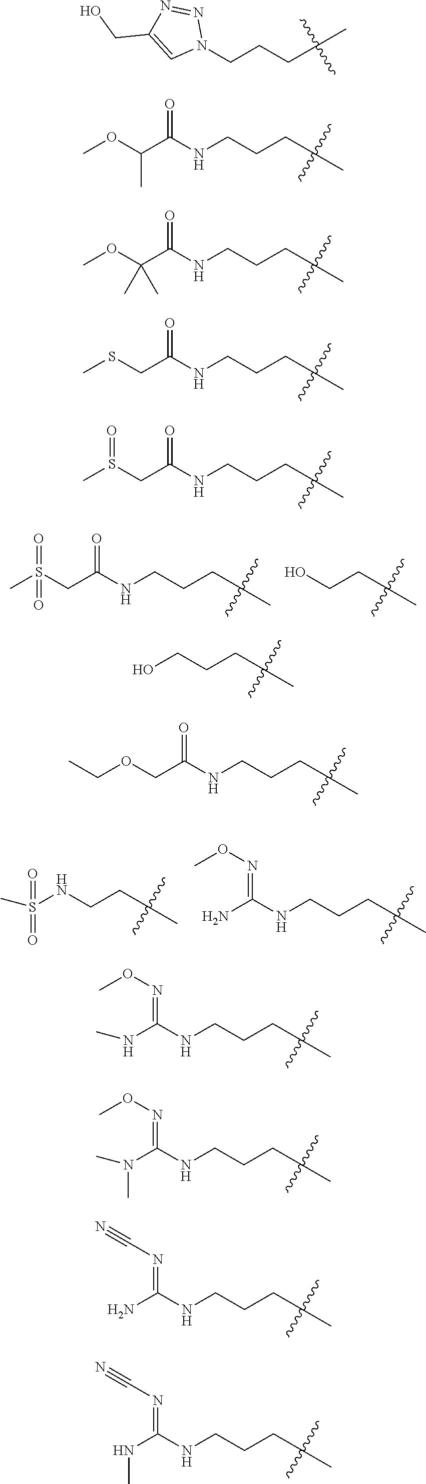

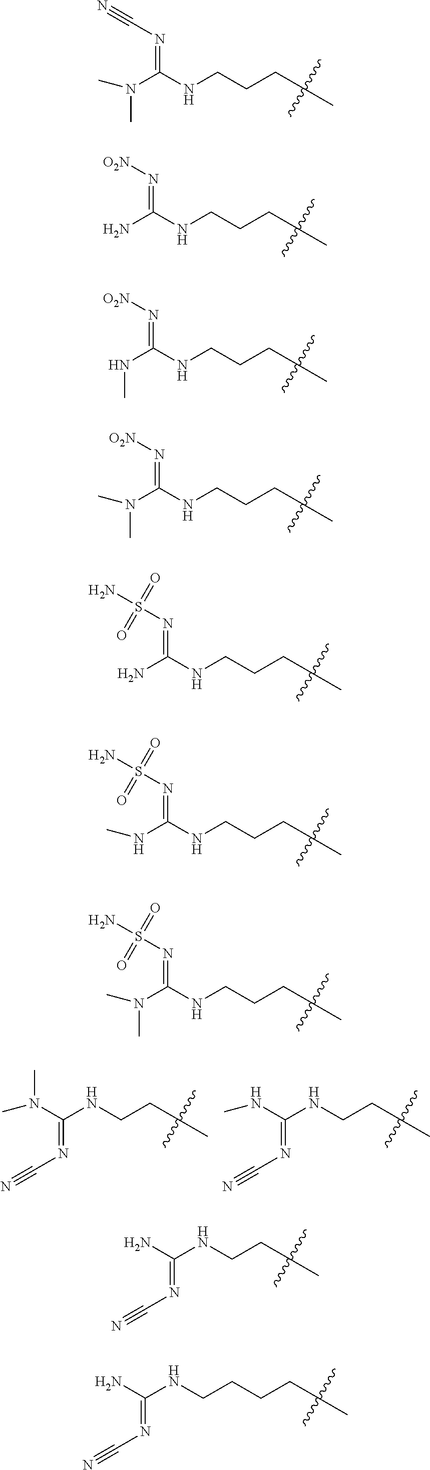

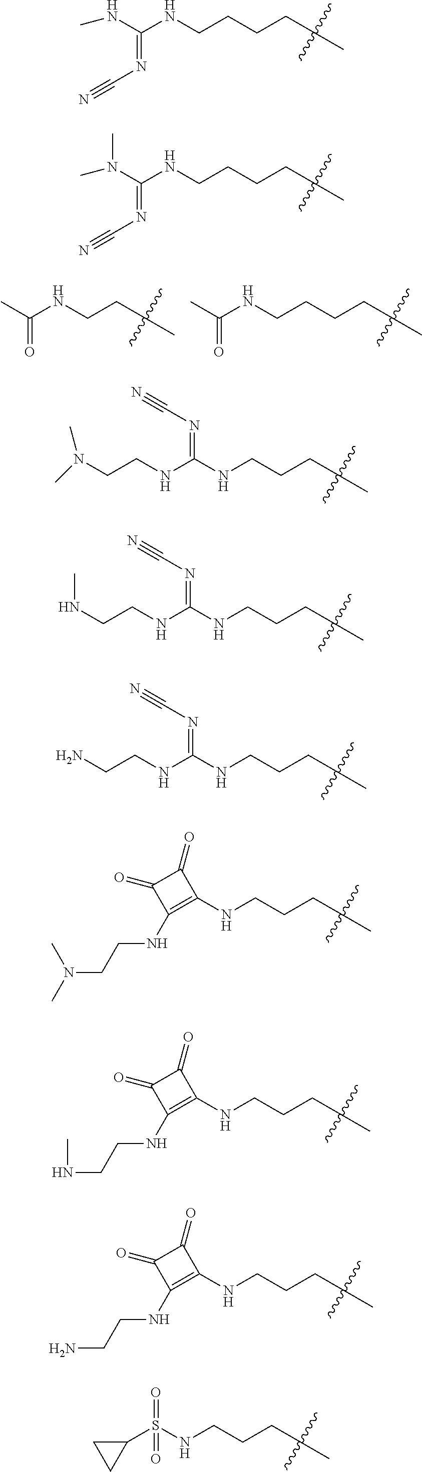

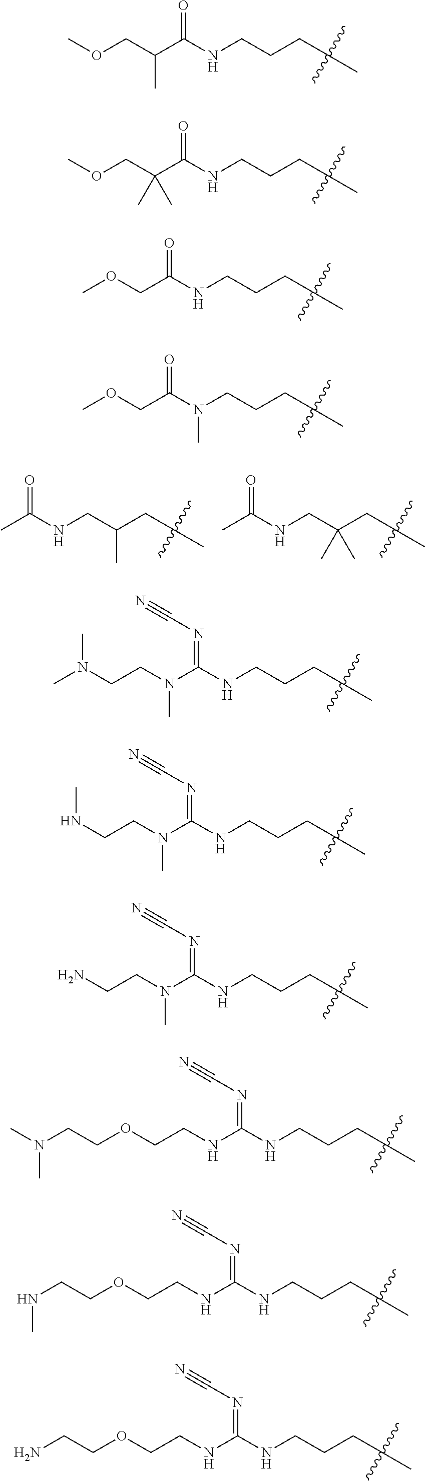

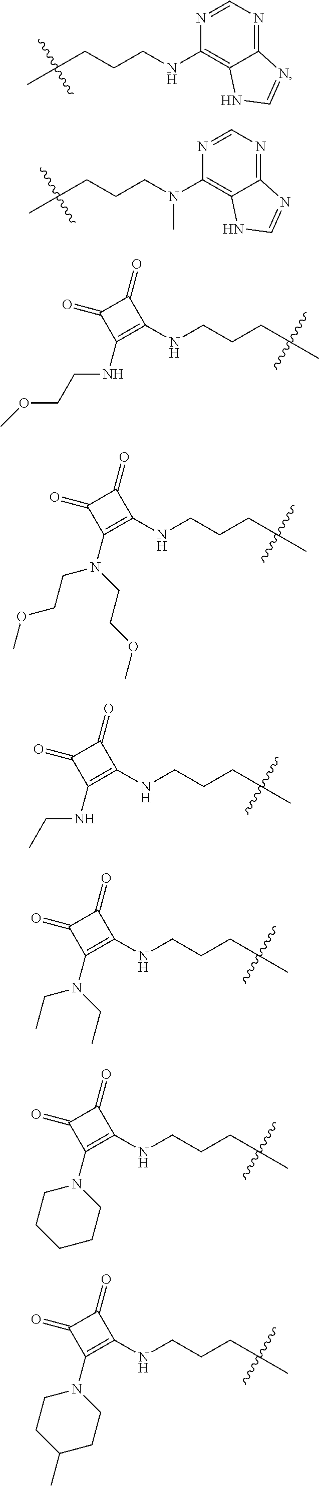

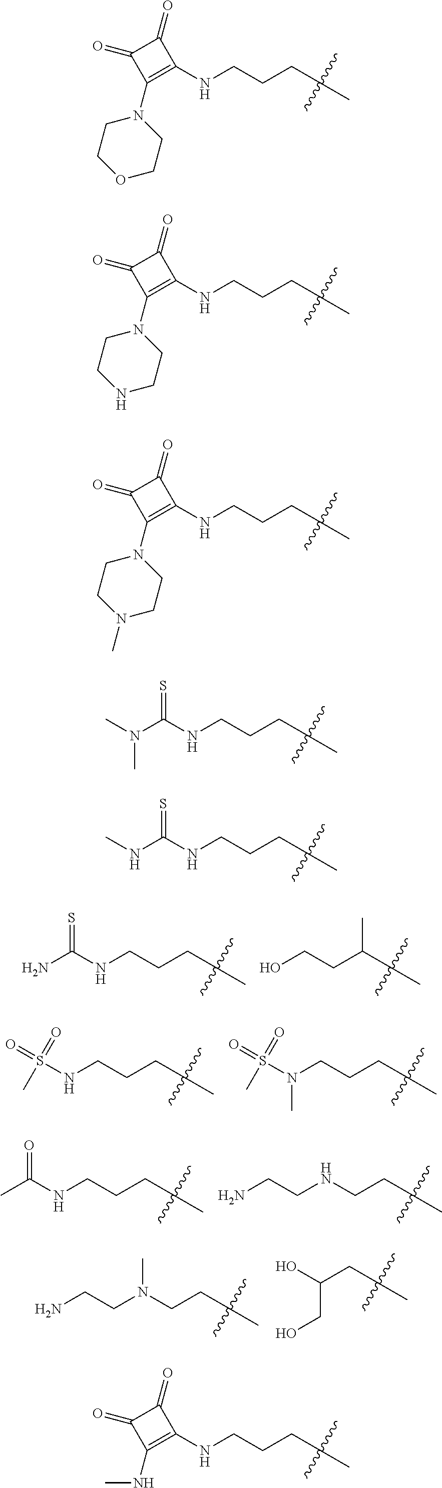

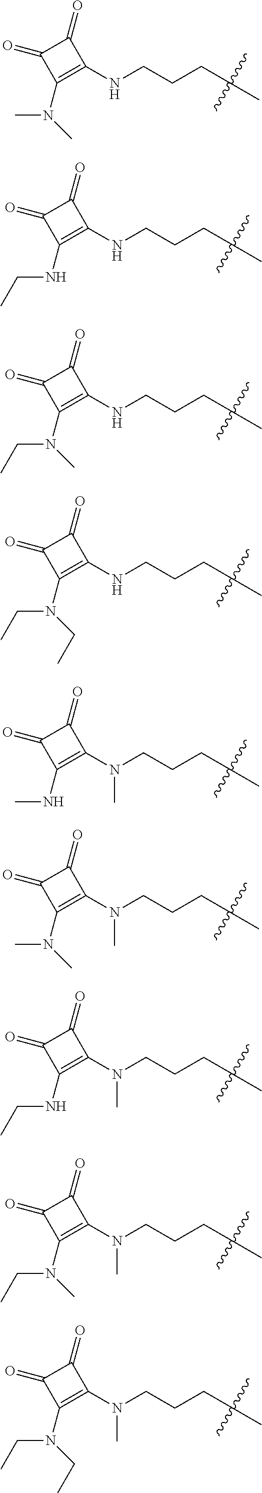

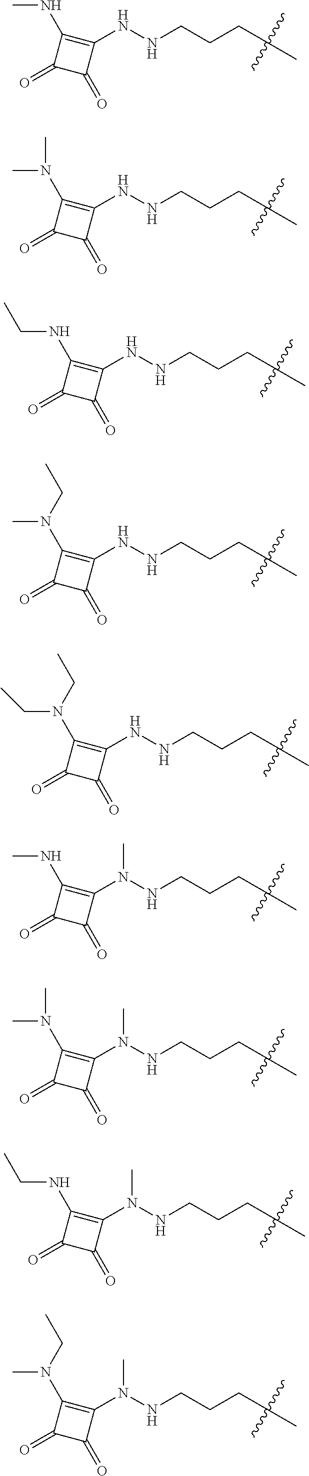

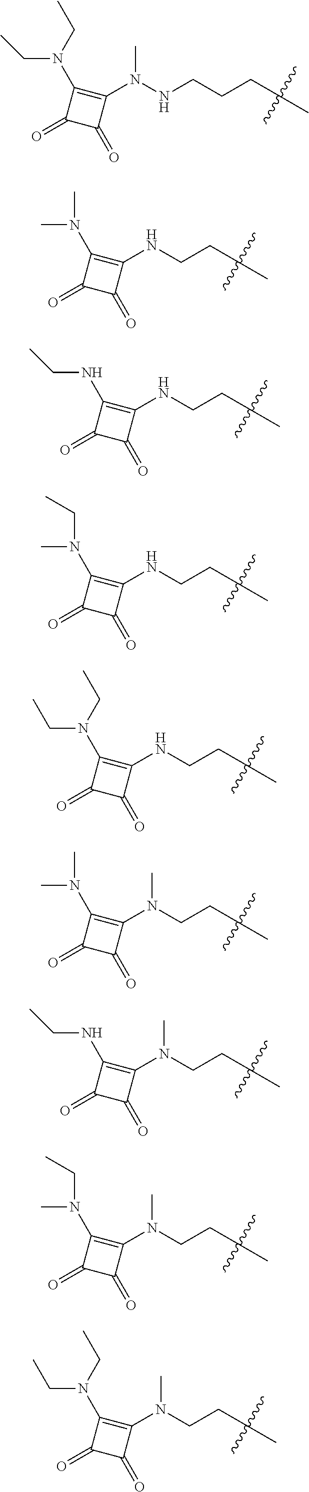

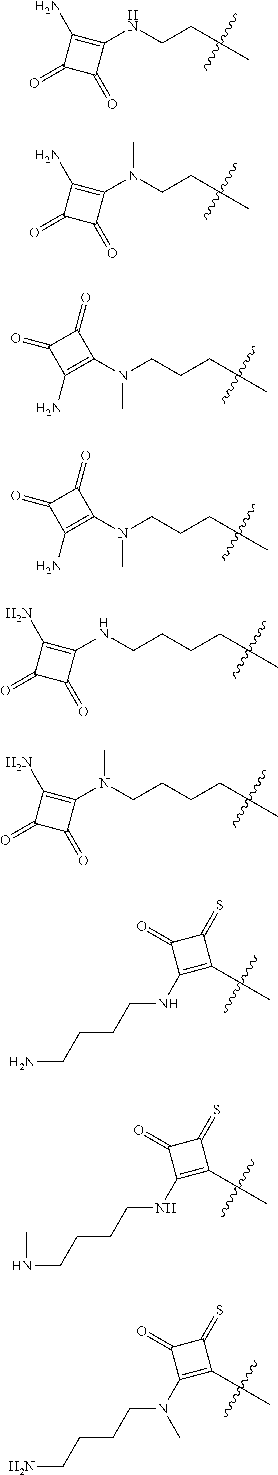

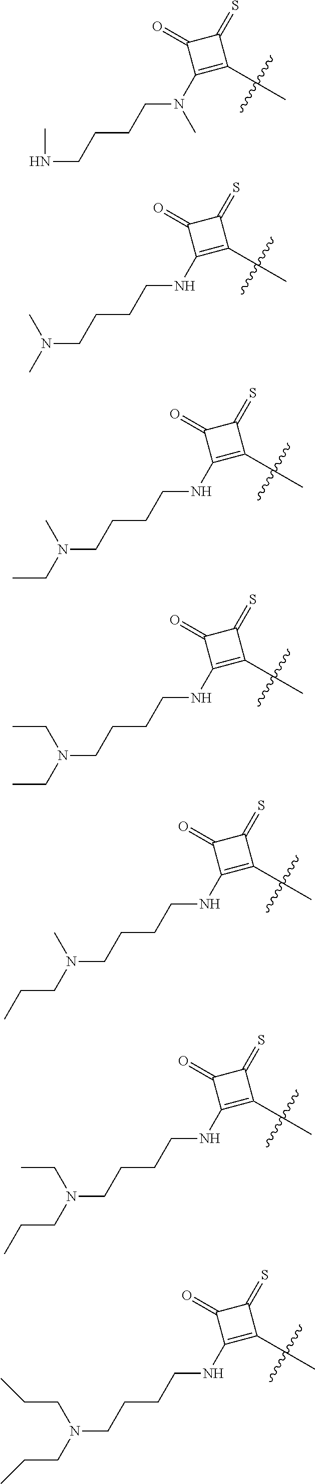

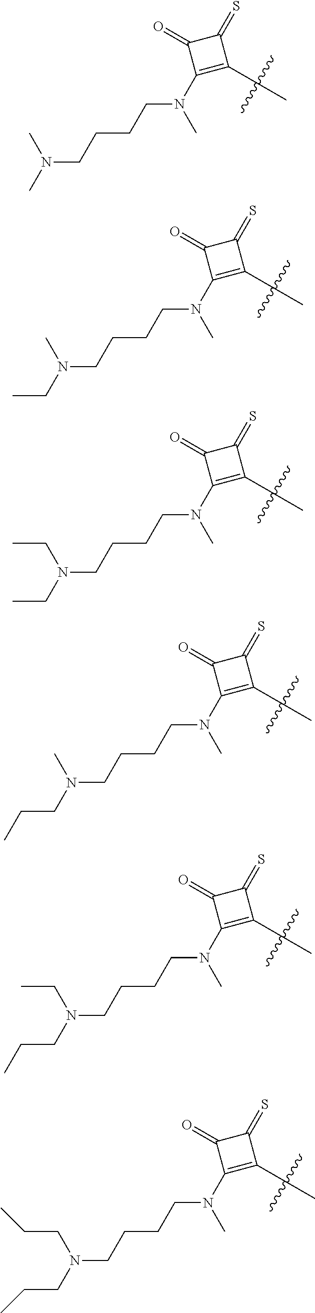 [SMILES: *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S]

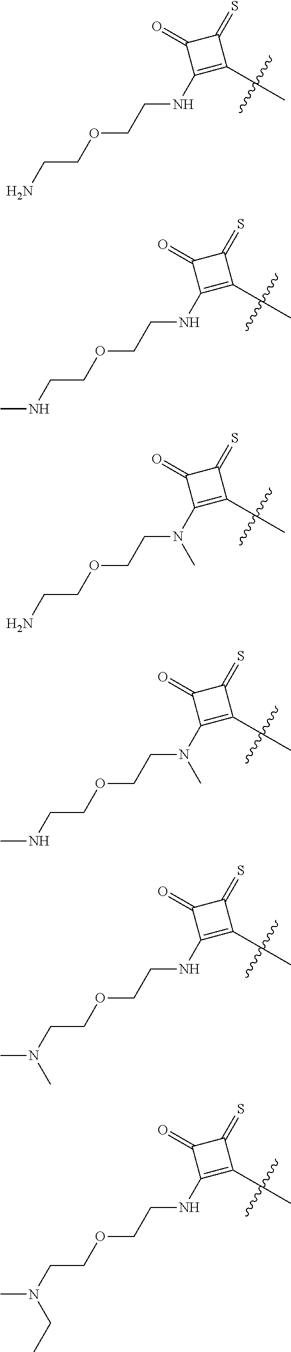 [SMILES: *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S ; *c1c(NCCOCCN(C)C)c(=O)c1=S ; *c1c(NCCOCCN(C)CC)c(=O)c1=S]

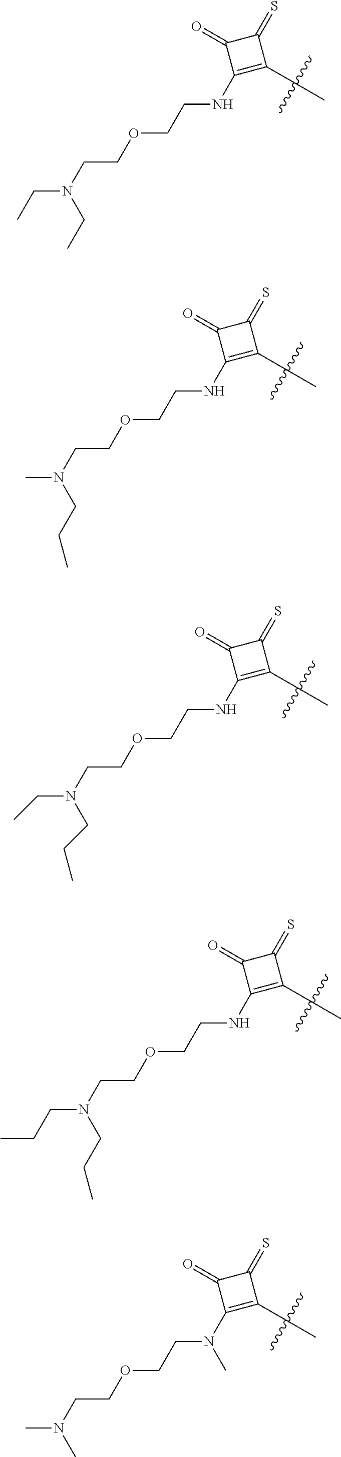 [SMILES: *c1c(NCCOCCN(CC)CC)c(=O)c1=S ; *c1c(NCCOCCN(C)CCC)c(=O)c1=S ; *c1c(NCCOCCN(CC)CCC)c(=O)c1=S ; *c1c(NCCOCCN(CCC)CCC)c(=O)c1=S ; *c1c(N(C)CCOCCN(C)C)c(=O)c1=S]

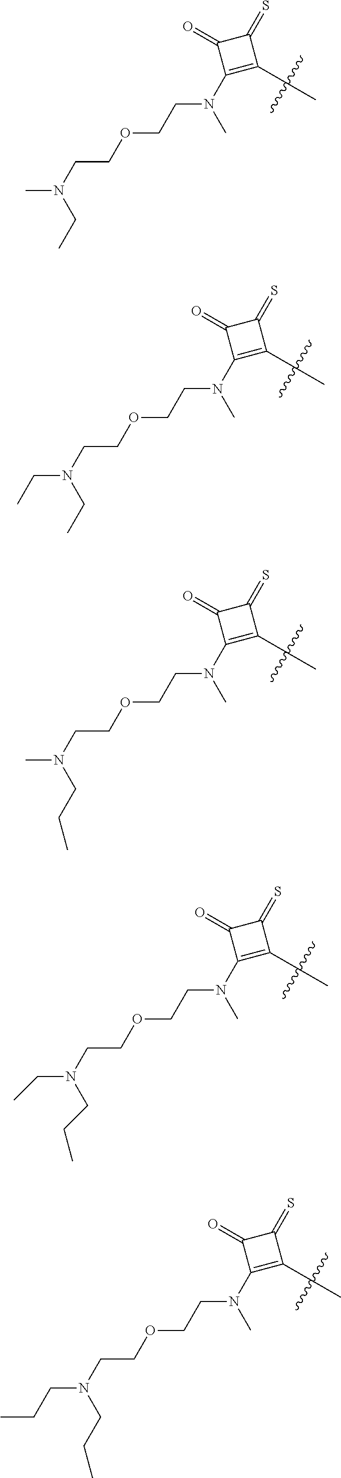 [SMILES: *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S ; *c1c(N(C)CCOCCN(CC)CCC)c(=O)c1=S ; *c1c(N(C)CCOCCN(CCC)CCC)c(=O)c1=S]

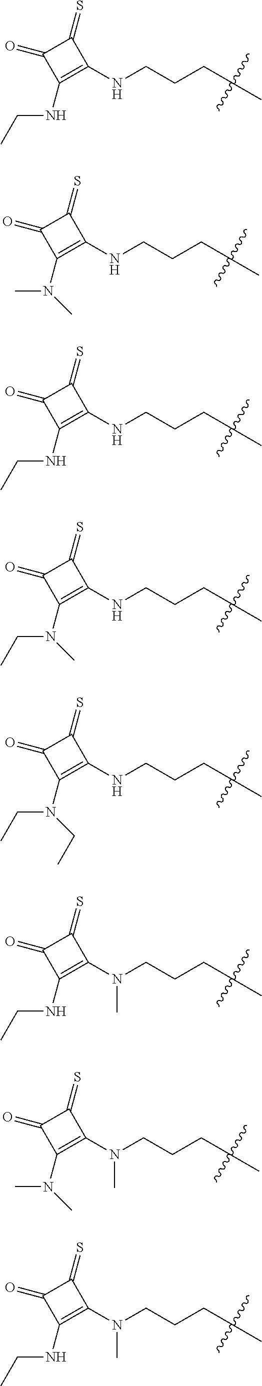

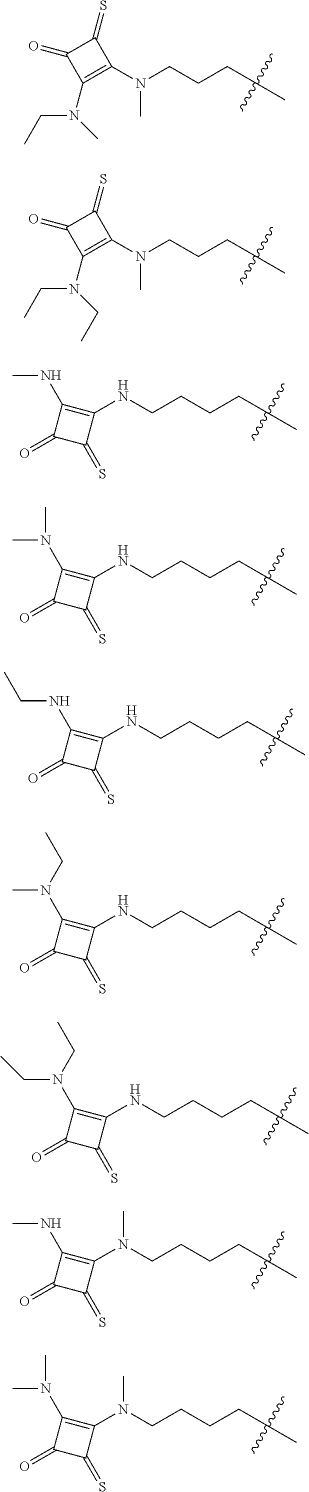

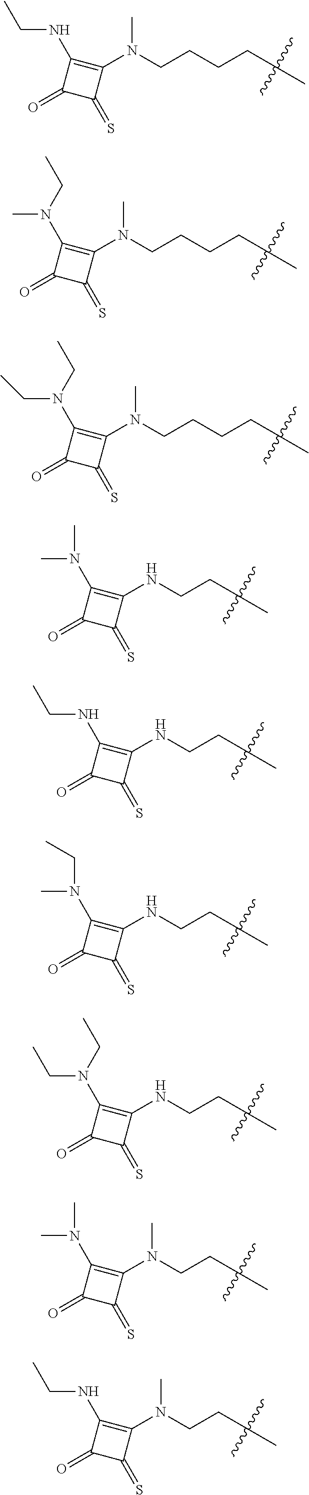

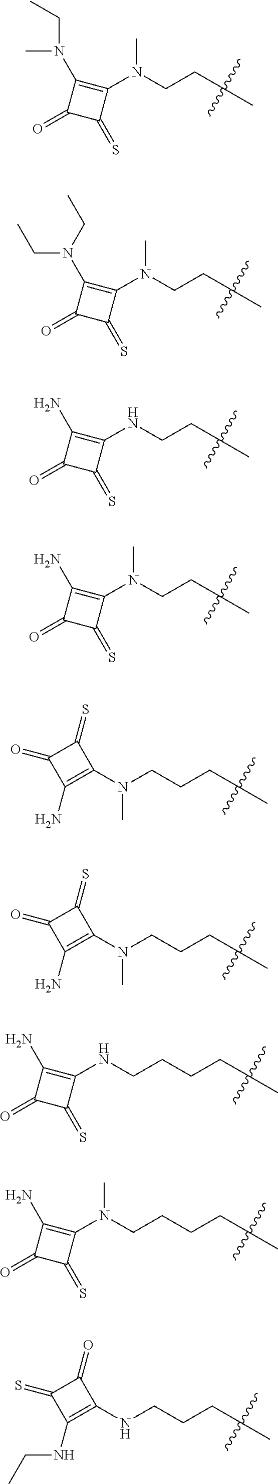

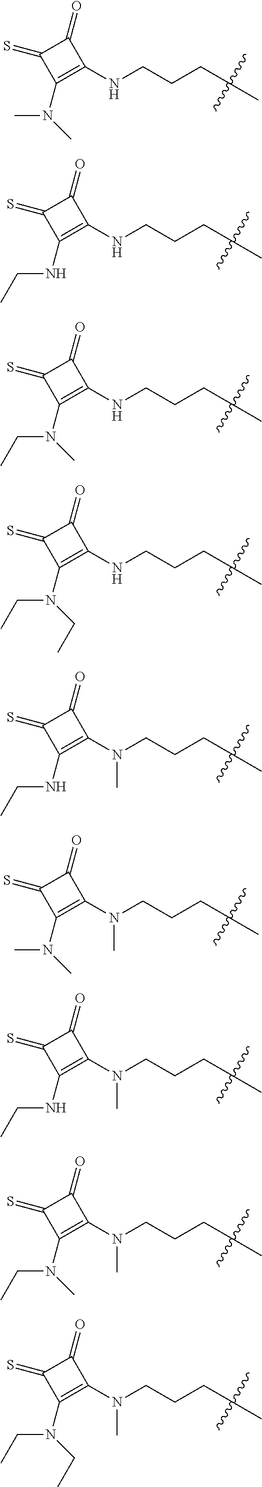

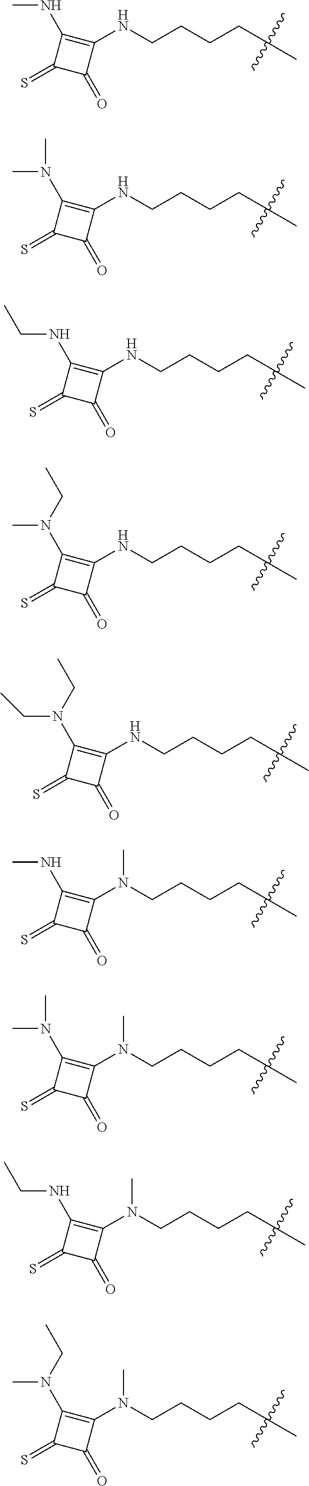

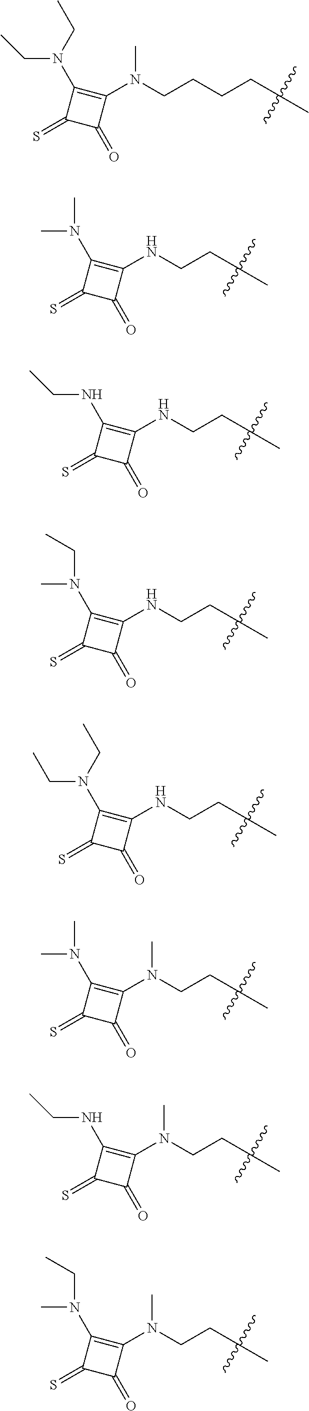

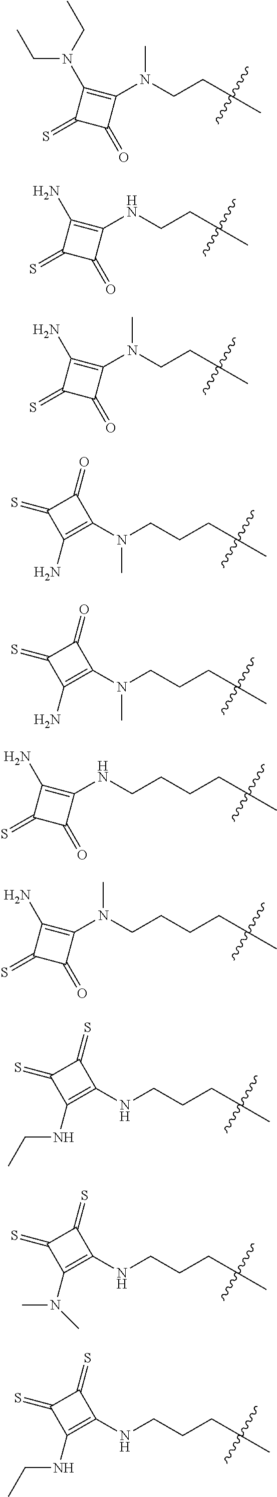

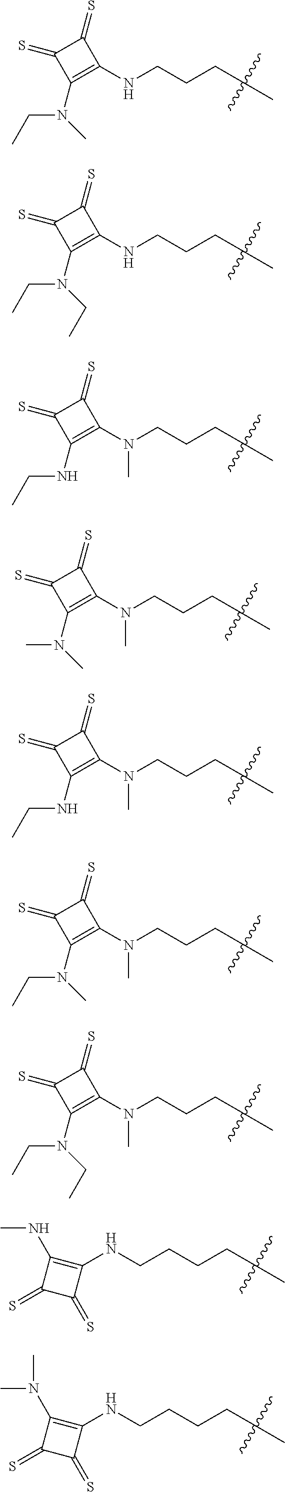

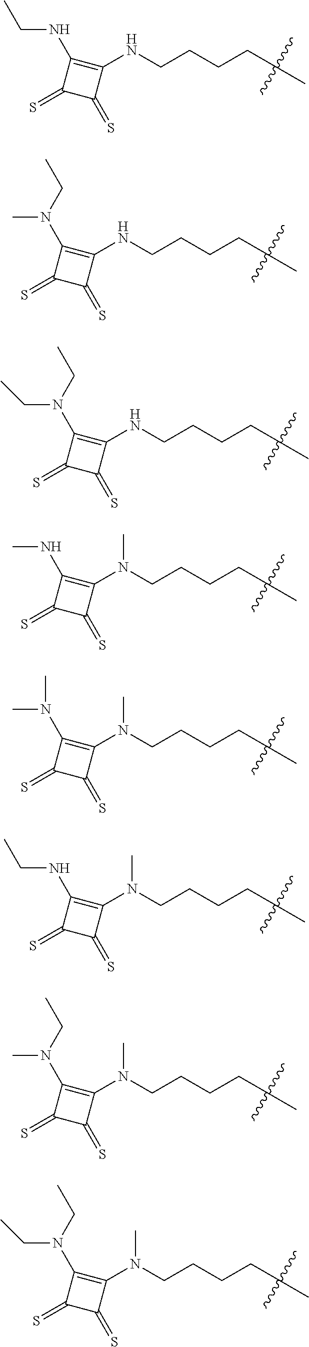

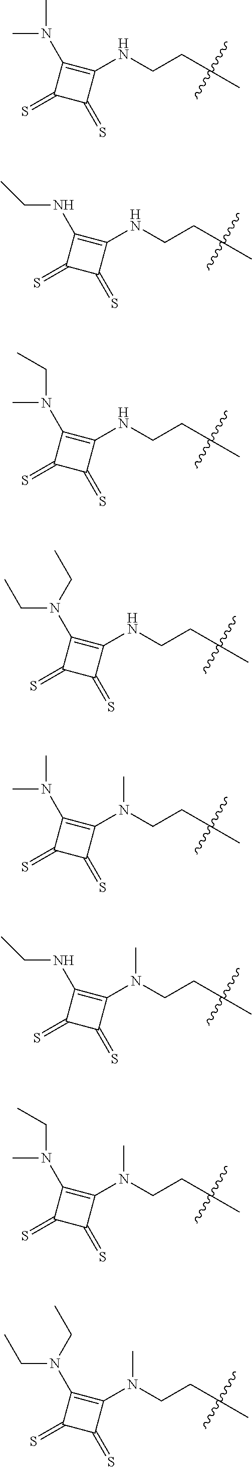

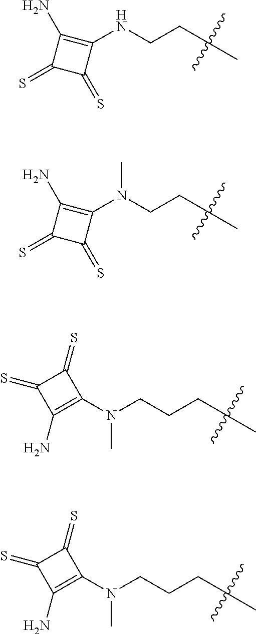 [SMILES: *CCNc1c(N)c(=S)c1=S ; *CCN(C)c1c(N)c(=S)c1=S ; *CCCN(C)c1c(N)c(=S)c1=S ; *CCCN(C)c1c(N)c(=S)c1=S]

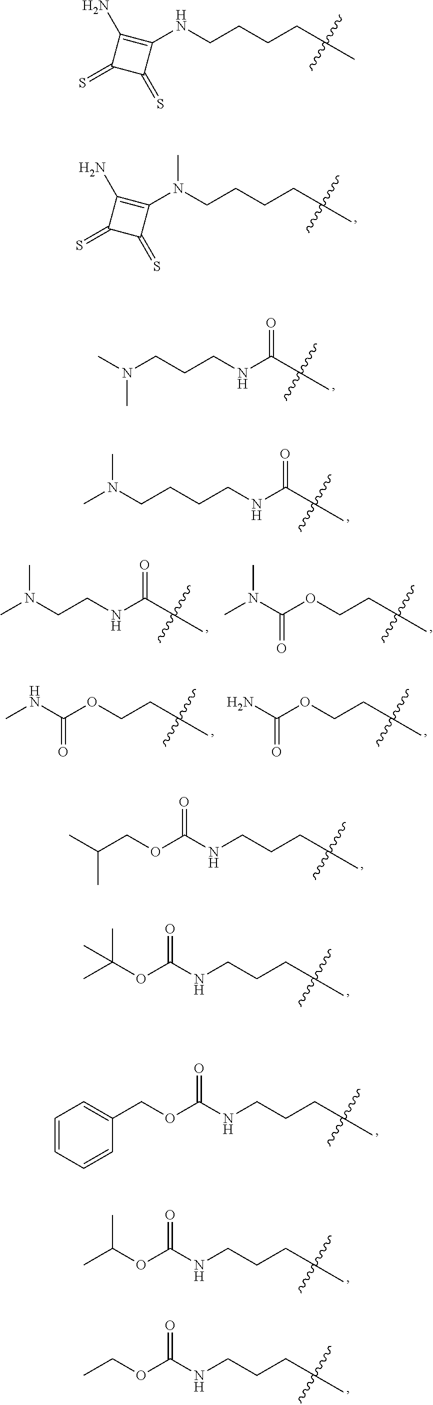

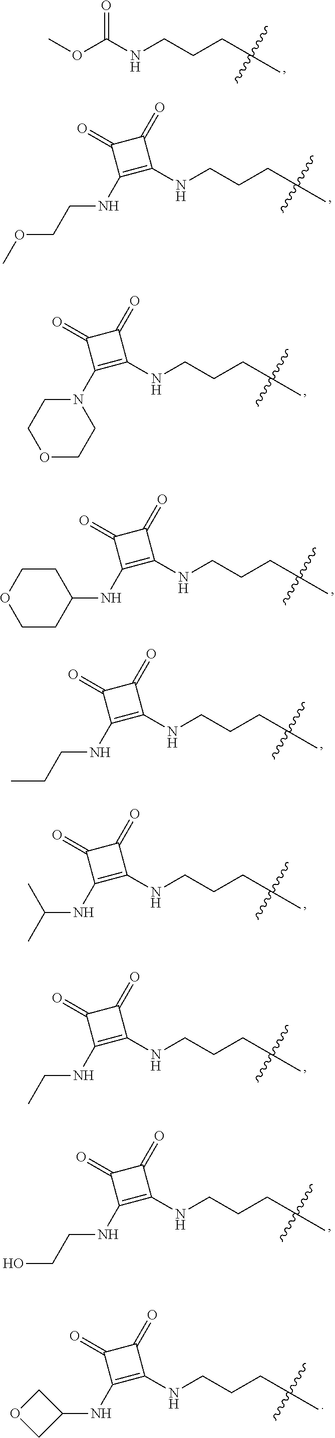

s1, s2, m1, m2, n1, p1, and p2

As defined herein, s1 is an integer from 4-10, inclusive. In certain embodiments, s1 is 4. In certain embodiments, s1 is 5. In certain embodiments, s1 is 6. In certain embodiments, s1 is 7. In certain embodiments, s1 is 8. In certain embodiments, s1 is 9. In certain embodiments, s1 is 10.

As defined herein, s2 is an integer from 4-10, inclusive. In certain embodiments, s2 is 4. In certain embodiments, s2 is 5. In certain embodiments, s2 is 6. In certain embodiments, s2 is 7. In certain embodiments, s2 is 8. In certain embodiments, s2 is 9. In certain embodiments, s2 is 10.

As defined herein, m1 is 0 or an integer from 1-10, inclusive. In certain embodiments, m1 is 0. In certain embodiments, m1 is 1. In certain embodiments, m1 is 2. In certain embodiments, m1 is 3. In certain embodiments, m1 is 4. In certain embodiments, m1 is 5. In certain embodiments, m1 is 6. In certain embodiments, m1 is 7. In certain embodiments, m1 is 8. In certain embodiments, m1 is 9. In certain embodiments, m1 is 10.

As defined herein, m2 is 0 or an integer from 1-10, inclusive. In certain embodiments, m2 is 0. In certain embodiments, m2 is 1. In certain embodiments, m2 is 2. In certain embodiments, m2 is 3. In certain embodiments, m2 is 4. In certain embodiments, m2 is 5. In certain embodiments, m2 is 6. In certain embodiments, m2 is 7. In certain embodiments, m2 is 8. In certain embodiments, m2 is 9. In certain embodiments, m2 is 10.

As defined herein, n1 is an integer from 1-10, inclusive. In certain embodiments, n1 is 1. In certain embodiments, n1 is 2. In certain embodiments, n1 is 3. In certain embodiments, n1 is 4. In certain embodiments, n1 is 5. In certain embodiments, n1 is 6. In certain embodiments, n1 is 7. In certain embodiments, n1 is 8. In certain embodiments, n1 is 9. In certain embodiments, n1 is 10.

As defined herein, p1 is 0 or an integer from 1-10, inclusive. In certain embodiments, p1 is 0. In certain embodiments, p1 is 1. In certain embodiments, p1 is 2. In certain embodiments, p1 is 3. In certain embodiments, p1 is 4. In certain embodiments, p1 is 5. In certain embodiments, p1 is 6. In certain embodiments, p1 is 7. In certain embodiments, p1 is 8. In certain embodiments, p1 is 9. In certain embodiments, p1 is 10.

As defined herein, p2 is 0 or an integer from 1-10, inclusive. In certain embodiments, p2 is 0. In certain embodiments, p2 is 1. In certain embodiments, p2 is 2. In certain embodiments, p2 is 3. In certain embodiments, p2 is 4. In certain embodiments, p2 is 5. In certain embodiments, p2 is 6. In certain embodiments, p2 is 7. In certain embodiments, p2 is 8. In certain embodiments, p2 is 9. In certain embodiments, p2 is 10.

R⁶ and R⁷

As defined herein, R⁶ hydrogen, halogen, or optionally substituted C₁₋₆ alkyl. In certain embodiments, R⁶ is hydrogen. In certain embodiments, R⁶ is halogen. In certain embodiments, R⁶ is —F. In certain embodiments, R⁶ is optionally substituted C₁₋₆ alkyl. In certain embodiments, R⁶ is unsubstituted C₁₋₆ alkyl. In certain embodiments, R⁶ is optionally substituted C₁₋₃ alkyl. In certain embodiments, R⁶ is unsubstituted C₁₋₃ alkyl.

In certain embodiments, R⁶ is C₁₋₆ alkyl substituted with one instance of —OR^{O}. In certain embodiments, R⁶ is C₁₋₆ alkyl substituted with one instance of —OH. In certain embodiments, R⁶ is C₁₋₃ alkyl substituted with one instance of —OR^{O}. In certain embodiments, R⁶ is C₁₋₃ alkyl substituted with one instance of —OH. In certain embodiments, R⁶ is —CH₂OH.

As defined herein, R⁷ hydrogen, halogen, or optionally substituted C₁₋₆ alkyl. In certain embodiments, R⁷ is hydrogen. In certain embodiments, R⁷ is halogen. In certain embodiments, R⁷ is —F. In certain embodiments, R⁷ is optionally substituted C₁₋₆ alkyl. In certain embodiments, R⁷ is unsubstituted C₁₋₆ alkyl. In certain embodiments, R⁷ is optionally substituted C₁₋₃ alkyl. In certain embodiments, R⁷ is unsubstituted C₁₋₃ alkyl.

In certain embodiments, R⁷ is C₁₋₆ alkyl substituted with one instance of —OR^{O}. In certain embodiments, R⁷ is C₁₋₆ alkyl substituted with one instance of —OH. In certain embodiments, R⁷ is C₁₋₃ alkyl substituted with one instance of —OR^{O}. In certain embodiments, R⁷ is C₁₋₃ alkyl substituted with one instance of —OH. In certain embodiments, R⁷ is —CH₂OH.

R^{O}, R^{N}, X, and n

As defined herein, each R^{O} is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or an oxygen protecting group. In certain embodiments, at least one R^{O} is hydrogen. In certain embodiments, at least one R^{O} is optionally substituted C₁₋₆ alkyl. In certain embodiments, at least one R^{O} is optionally substituted C₂₋₆ alkenyl. In certain embodiments, at least one R^{O} is optionally substituted C₂₋₆ alkynyl. In certain embodiments, at least one R^{O} is optionally substituted C₃₋₈ carbocyclyl. In certain embodiments, at least one R^{O} is optionally substituted C₆₋₁₀ aryl. In certain embodiments, at least one R^{O} is optionally substituted C₃₋₈ heterocyclyl. In certain embodiments, at least one R^{O} is optionally substituted C₅₋₁₀ heteroaryl. In certain embodiments, at least one R^{O} is optionally substituted C₁₋₆ acyl. In certain embodiments, at least one R^{O} is an oxygen protecting group.

In certain embodiments, at least one R^{O} is unsubstituted C₁₋₆ alkyl. In certain embodiments, at least one R^{O} is unsubstituted C₁₋₃ alkyl. In certain embodiments, at least one R^{O} is methyl, ethyl, n-propyl, isopropyl, n-butyl, sec-butyl, iso-butyl, or tert-butyl.

As defined herein, each R^{N} is independently hydrogen, —OR^{O}, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or a nitrogen protecting group, or two R^{N} attached to the same nitrogen atoms are joined together with the intervening atoms to form optionally substituted heterocyclyl or optionally substituted heteroaryl. In certain embodiments, at least one R^{N} is hydrogen. In certain embodiments, at least one R^{N} is —OR^{O}. In certain embodiments, at least one R^{N} is optionally substituted C₁₋₆ alkyl. In certain embodiments, at least one R^{N} is optionally substituted C₂₋₆ alkenyl. In certain embodiments, at least one R^{N} is optionally substituted C₂₋₆ alkynyl. In certain embodiments, at least one R^{N} is optionally substituted C₃₋₈ carbocyclyl. In certain embodiments, at least one R^{N} is optionally substituted C₆₋₁₀ aryl. In certain embodiments, at least one R^{N} is optionally substituted C₃₋₈ heterocyclyl. In certain embodiments, at least one R^{N} is optionally substituted C₅₋₁₀ heteroaryl. In certain embodiments, at least one R^{N} is optionally substituted C₁₋₆ acyl. In certain embodiments, at least one R^{N} is a nitrogen protecting group.

In certain embodiments, at least one R^{N} is unsubstituted C₁₋₆ alkyl. In certain embodiments, at least one R^{N} is unsubstituted C₁₋₃ alkyl. In certain embodiments, at least one R^{N} is methyl, ethyl, n-propyl, isopropyl, n-butyl, sec-butyl, iso-butyl, or tert-butyl.

In certain embodiments, two R^{N} attached to the same nitrogen atoms are joined together with the intervening atoms to form optionally substituted heterocyclyl. In certain embodiments, two R^{N} attached to the same nitrogen atoms are joined together with the intervening atoms to form optionally substituted heterocyclyl optionally substituted heteroaryl.

As defined herein, R⁵ is hydrogen, —CN, —NO₂, —OR^{O}, —S(O)₂R, —S(O)₂ (R^{N})₂, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, or optionally substituted C₁₋₆ acyl, or optionally a nitrogen protecting group when attached to a nitrogen atom. In certain embodiments, R⁵ is hydrogen. In certain embodiments, R⁵ is —CN. In certain embodiments, R⁵ is —NO₂. In certain embodiments, R⁵ is —OR^{O}. In certain embodiments, R⁵ is-S(O)₂R. In certain embodiments, R⁵ is-S(O)₂ (R^{N})₂. In certain embodiments, R⁵ is optionally substituted C₁₋₆ alkyl. In certain embodiments, R⁵ is optionally substituted C₂₋₆ alkenyl. In certain embodiments, R⁵ is optionally substituted C₂₋₆ alkynyl. In certain embodiments, R⁵ is optionally substituted C₃₋₈ carbocyclyl. In certain embodiments, R⁵ is optionally substituted C₆₋₁₀ aryl. In certain embodiments, R⁵ is optionally substituted C₃₋₈ heterocyclyl. In certain embodiments, R⁵ is optionally substituted C₅₋₁₀ heteroaryl. In certain embodiments, R⁵ is or optionally substituted C₁₋₆ acyl. In certain embodiments, R⁵ is a nitrogen protecting group.

As defined herein, each X is independently halogen. In certain embodiments, at least one X is —I. In certain embodiments, at least one X is —Br. In certain embodiments, at least one X is —Cl. In certain embodiments, at least one X is —F.

As defined herein n is 1, 2, 3, 4, or 5. In certain embodiments, n is 1. In certain embodiments, n is 2. In certain embodiments, n is 3. In certain embodiments, n is 4. In certain embodiments, n is 5.

### Lipid Nanoparticles (LNPs)

Also provided herein are nanoparticles (e.g., lipid nanoparticles (LNPs)) comprising at least one ionizable lipid (e.g., a compound of Formula (I), (I-a), (I-b), (I-c), (I-d), (I-e), or (I-f)) described herein.

Nanoparticle compositions include, for example, lipid nanoparticles (LNPs; e.g., empty LNPs or loaded LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or cross-linked to one another. Lipid bilayers may include one or more ligands, proteins, or channels. In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method.

Lipid nanoparticle (LNP) compositions provided herein comprise a lipid component including at least one ionizable lipid (e.g., a compound of Formula (I), (I-a), (I-b), (I-c), (I-d), (I-e), or (I-f)) described herein. For example, the lipid component of an LNP composition may include one or more of lipids of Table 1. LNP compositions may also include a variety of other components. For example, the lipid component of the LNP composition may include one or more other lipids in addition to an ionizable lipid (e.g., a compound of Formula (I), (I-a), (I-b), (I-c), (I-d), (I-e), or (I-f)). Other lipids include, without limitation, other cationic/ionizable lipids, phospholipids, structural lipids, and PEG lipids.

Lipid nanoparticle (LNP) compositions provided herein comprise a lipid component including at least one ionizable lipid (e.g., a compound of Formula (I), (I-a), (I-b), (I-c), (I-d), (I-e), or (I-f)) described herein. For example, the lipid component of an LNP composition may include one or more of lipids of Table 1. LNP compositions may also include a variety of other components. For example, the lipid component of the LNP composition may include one or more other lipids in addition to an ionizable lipid (e.g., a compound of Formula (I), (I-a), (I-b), (I-c), (I-d), (I-e), or (I-f)). Other lipids include, without limitation, other cationic/ionizable lipids, phospholipids, structural lipids, and PEG lipids.

### Cationic/Ionizable Lipids

The LNP may include one or more cationic and/or ionizable lipids (e.g., lipids that may have a positive or partial positive charge at physiological pH) in addition to an ionizable lipid (e.g., a compound of Formula (I), (I-a), (I-b), (I-c), (I-d), (I-e), or (I-f)) described herein. Cationic and/or ionizable lipids may be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, a cationic lipid may also be a lipid including a cyclic amine group.

Other examples of cationic/ionizable amino lipids can be found in, e.g., International PCT Application Publication Nos. WO 2017/049245, published Mar. 23, 2017; WO 2017/112865, published Jun. 29, 2017; WO 2018/170306, published Sep. 20, 2018; WO 2018/232120, published Dec. 20, 2018; WO 2020/061367, published Mar. 26, 2020; WO 2021/055835, published Mar. 25, 2021; WO 2021/055833, published Mar. 25, 2021; WO 2021/055849, published Mar. 25, 2021; and WO 2022/204288, published Sep. 29, 2022, the entire contents of each of which (including any generic or specific structures disclosed therein) is incorporated herein by reference.

### Structural Lipids

The LNP may include one or more structural lipids. In certain embodiments, the structural lipid is a steroid. Structural lipids can be selected from the group consisting of, but are not limited to, cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof. In some embodiments, the structural lipid is:

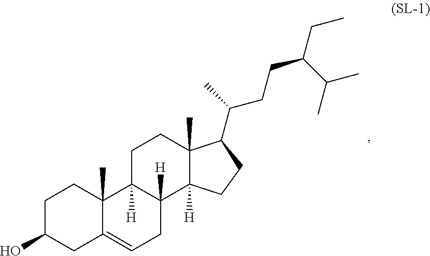

or a pharmaceutically acceptable salt thereof.

### Phospholipids

The LNP may include one or more phospholipids, such as one or more (poly) unsaturated phospholipids. In many instances, phospholipids can assemble into one or more lipid bilayers. In general, phospholipids may include a phospholipid moiety and one or more fatty acid moieties. For example, a phospholipid may be a lipid according to Formula (H):

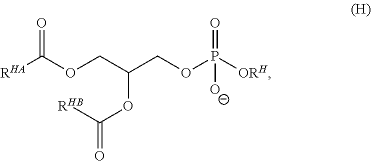

or a pharmaceutically acceptable salt thereof, wherein:
R^{H} is a phospholipid moiety;
and R^{A} and R^{B} are independently fatty acid moieties with or without unsaturation that may be the same or different.

A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of an LNP to facilitate membrane permeation or cellular recognition or in conjugating an LNP to a useful component such as a targeting or imaging moiety (e.g., a dye).

Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), and mixtures thereof. In some embodiments, an LNP includes DSPC. In certain embodiments, an LNP includes DOPE. In some embodiments, an LNP includes both DSPC and DOPE.

### PEG Lipids

The LNP may include one or more PEG lipids or PEG-modified lipids. Such species may be alternately referred to as PEGylated lipids. A PEG lipid is a lipid modified with polyethylene glycol unit. A PEG lipid may be selected from the non-limiting group consisting of PEG-modified phosphatidylethanolamines, PEG-modified phosphatidic acids, PEG-modified ceramides (PEG-CER), PEG-modified dialkylamines, PEG-modified diacylglycerols (PEG-DEG), PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In certain embodiments, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

In certain embodiments, PEG lipid is selected from the group consisting of 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). For example, in some embodiments, the PEG lipid is PEG-DMG.

In certain embodiments, the PEG lipid is a compound of Formula (PL-I):

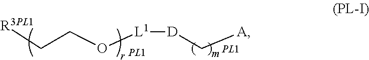

or a pharmaceutically acceptable salt thereof, wherein:
R^{3PL1} is —OR^{OPL1};
R^{OPL1} is hydrogen, optionally substituted alkyl, or an oxygen protecting group;
r^{PL1} is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, —O—, —N(R^{NPL1})—, —S—, —C(O)—, —C(O)N(R^{NPL1})—, —NR^{NPL1}C(O)—, —C(O)O—, —OC(O)—, —OC(O)O—, —OC(O)N(R^{NPL1})—, —NR^{NPL1}C(O)O—, or —NR^{NPL1}C(O)N(R^{NPL1})—;
D is a moiety obtained by click chemistry or a moiety cleavable under physiological conditions (e.g., an ester);
m^{PL1} is 0, 1, 2, 3, 4, 5, 6, 7, 8, 9, or 10;
A is of the formula:

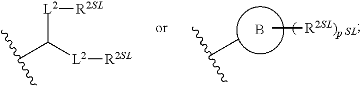

each instance of L² is independently a bond or optionally substituted C₁₋₆ alkylene, wherein one methylene unit of the optionally substituted C₁₋₆ alkylene is optionally replaced with —O—, —N(R^{NPL1})—, —S—, —C(O)—, —C(O)N(R^{NPL1})—, —NR^{NPL1}C(O)—, —C(O)O—, —OC(O)—, —OC(O)O—, —OC(O)N(R^{NPL1})—, —NR^{NPL1}C(O)O—, or —NR^{NPL1}C(O)N(R^{NPL1})—;
each instance of R²SL is independently optionally substituted C₁₋₃₀ alkyl, optionally substituted C₁₋₃₀ alkenyl, or optionally substituted C₁₋₃₀ alkynyl; optionally wherein one or more methylene units of R^{2SL} are independently replaced with optionally substituted carbocyclylene, optionally substituted heterocyclylene, optionally substituted arylene, optionally substituted heteroarylene, —N(R^{NPL1})—, —O—, —S—, —C(O)—, —C(O)N(R^{NPL1})—, —NR^{NPL1}C(O)—, —NR^{NPL1}C(O)N(R^{NPL1})—, —C(O)O—, —OC(O)—, —OC(O)O—, —OC(O)N(R^{NPL1})—, —NR^{NPL1}C(O)O—, —C(O)S—, —SC(O)—, —C(═NR^{NPL1})—, —C(═NR^{NPL1})N(R^{NPL1})—, —NR^{NPL1}C(═NR^{NPL1})—, —NR^{NPL1}C(═NR^{NPL1})N(R^{NPL1})—, —C(S)—, —C(S)N(R^{NPL1})—, —NR^{NPL1}C(S)—, —NR^{NPL1}C(S)N(R^{NPL1})—, —S(O)—, —OS(O)—, —S(O)O—, —OS(O)O—, —OS(O)₂—, —S(O)₂O—, —OS(O)₂O—, —N(R^{NPL1})S(O)—, —S(O)N(R^{NPL1})—, —N(R^{NPL1})S(O)N(R^{NPL1})—, —OS(O)N(R^{NPL1})—, —N(R^{NPL1})S(O)O—, —S(O)₂—, —N(R^{NPL1})S(O)₂—, —S(O)₂N(R^{NPL1})—, —N(R^{NPL1})S(O)₂N(R^{NPL1})—, —OS(O)₂N(R^{NPL1})—, or —N(R^{NPL1})S(O)₂O—;
   each instance of R^{NPL1} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p^{SL} is 1 or 2.

In certain embodiments, the PEG lipid is a compound of Formula (PL-I-OH):

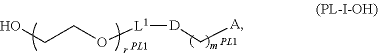

or a pharmaceutically acceptable salt thereof.

In certain embodiments, the PEG lipid is a compound of Formula (PL-II-OH):

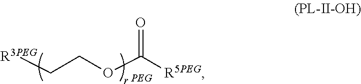

or a pharmaceutically acceptable salt thereof, wherein:
R^{3PEG} is —OR^{O};
R^{O} is hydrogen, C₁₋₆ alkyl or an oxygen protecting group;
r^{PEG} is an integer between 1 and 100;
R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C3-10 carbocyclylene, 4 to 10 membered heterocyclylene, C₆₋₁₀ arylene, 4 to 10 membered heteroarylene, —N(R^{NPEG})—, —O—, —S—, —C(O)—, —C(O)N(R^{NPEG})—, —NR^{NPEG}C(O)—, —NR^{NPEG}C(O)N(R^{NPEG})—, —C(O)O—, —OC(O)—, —OC(O)O—, —OC(O)N(R^{NPEG})—, —NR^{NPEG}C(O)O—, —C(O)S—, —SC(O)—, —C(═NR^{NPEG})—, —C(═NR^{NPEG})N(R^{NPEG})—, —NR^{NPEG}C(═NR^{NPEG})—, —NR^{NPEG}C(═NR^{NPEG})N(R^{NPEG})—, —C(S)—, —C(S)N(R^{NPEG})—, —NR^{NPEG}C(S)—, —NR^{NPEG}C(S)N(R^{NPEG})—, —S(O)—, —OS(O)—, —S(O)O—, —OS(O)O—, —OS(O)₂—, —S(O)₂O—, —OS(O)₂O—, —N(R^{NPEG})S(O)—, —S(O)N(R^{NPEG})—, —N(R^{NPEG})S(O)N(R^{NPEG})—, —OS(O)N(R^{NPEG})—, —N(R^{NPEG}S(O)O—, —S(O)₂—, —N(R^{NPEG}) S(O)₂—, —S(O)₂N(R^{NPEG})—, —N(R^{NPEG}) S(O)₂N(R^{NPEG})—, —OS(O)₂N(R^{NPEG})—, or —N(R^{NPEG})S(O)₂O—; and
each instance of R^{NPEG} is independently hydrogen, C₁₋₆ alkyl, or a nitrogen protecting group.

In certain embodiments, in the PEG lipid of Formula (PL-II-OH), r is an integer between 40 and 50. For example, r is selected from the group consisting of 40, 41, 42, 43, 44, 45, 46, 47, 48, 49 and 50. For example, r is 45.

In certain embodiments, in the PEG lipid of Formula (PL-II-OH), R⁵ is C₁₇ alkyl.

In certain embodiments, the PEG lipid is a compound of one of the following formulae:

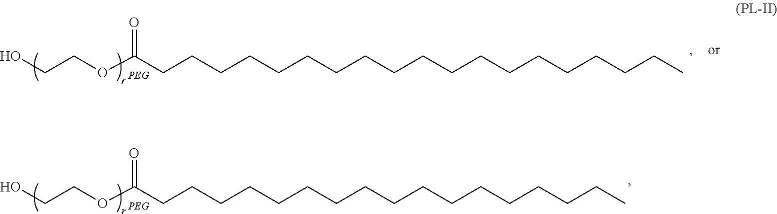

wherein r^{PEG} is an integer between 1 and 100.

In certain embodiments, the PEG lipid is a compound of Formula (PEG-1):

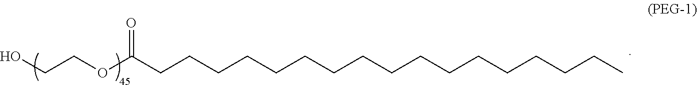

In certain embodiments, the PEG lipid is a compound of Formula (PL-III):

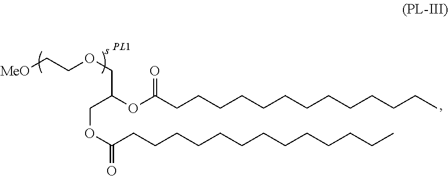

or a salt or isomer thereof, wherein s^{PL1} is an integer between 1 and 100.

In certain embodiments, the PEG lipid is a compound of following formula:

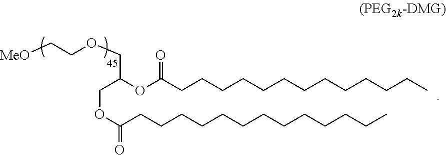

In certain embodiments, the incorporation of a PEG lipid of one of formulae (PL-I), PL-I—OH), (PL-II), (PL-II-OH), (PL-III), (PEG₂ₖ-DMG), or (PEG-1) in the LNP formulation can improve the pharmacokinetics and/or biodistribution of the LNP formulation. For example, incorporation of a PEG lipid of one of formulae (PL-II-OH), (PL-IIa-OH), (PL-II), or (PEG-1) in the LNP formulation can reduce the accelerated blood clearance (ABC) effect.

Other non-limiting examples of PEG lipids can be found in, e.g., International PCT Application Publication Nos. WO 2020/061284, published Mar. 26, 2020; and WO 2020/061295, published Mar. 26, 2020, the entire contents of each of which (including any generic or specific structures disclosed therein) is incorporated herein by reference.

### Adjuvants

In some embodiments, an LNP that includes one or more lipids described herein may further include one or more adjuvants, e.g., Glucopyranosyl Lipid Adjuvant (GLA), CpG oligodeoxynucleotides (e.g., Class A or B), poly(I:C), aluminum hydroxide, Pam3CSK4.

Therapeutic and/or Prophylactic Agents

LNPs may include one or more therapeutic and/or prophylactic agents. The disclosure features methods of delivering a therapeutic and/or prophylactic agent to a cell or organ, producing a polypeptide of interest in a cell, and treating or preventing a disease or disorder in a subject in need thereof, the method comprising administering to a subject and/or contacting a cell with an LNP comprising a therapeutic and/or prophylactic agent described herein. In some aspects, the disclosure features methods of delivering a therapeutic and/or prophylactic agent to a mammalian cell or organ, producing a polypeptide of interest in a mammalian cell, and treating or preventing a disease or disorder in a mammal in need thereof, the methods comprising administering to a mammal and/or contacting a mammalian cell with an LNP comprising a therapeutic and/or prophylactic agent described herein.

Therapeutic and/or prophylactic agents include biologically active substances and are alternately referred to as “active agents.” A therapeutic and/or prophylactic agent may be a substance that, once delivered to a cell or organ, brings about a desirable change in the cell, organ, or other bodily tissue or system. Such species may be useful in the treatment of one or more diseases, disorders, or conditions. In some embodiments, a therapeutic and/or prophylactic agent is a small molecule drug useful in the treatment of a particular disease, disorder, or condition.

In some embodiments, a therapeutic and/or prophylactic agent is a vaccine, a compound (e.g., a polynucleotide or nucleic acid molecule that encodes a protein or polypeptide) that elicits an immune response, and/or another therapeutic and/or prophylactic agent. Vaccines include compounds and preparations that are capable of providing immunity against one or more conditions related to infectious diseases and can include mRNAs encoding infectious disease derived antigens and/or epitopes. Vaccines also include compounds and preparations that direct an immune response against cancer cells and can include mRNAs encoding tumor cell derived antigens, epitopes, and/or neoepitopes. In some embodiments, a vaccine and/or a compound capable of eliciting an immune response is administered intramuscularly via a composition of the disclosure.

In other embodiments, a therapeutic and/or prophylactic agent is a protein, for example a protein needed to augment or replace a naturally-occurring protein of interest. Such proteins or polypeptides may be naturally occurring, or may be modified using methods known in the art, e.g., to increase half life. Exemplary proteins are intracellular, transmembrane, or secreted.

### Polynucleotides and Nucleic Acids

In some embodiments, a therapeutic and/or prophylactic agent is a nucleic acid. In some embodiments, a therapeutic and/or prophylactic agent is selected from ribonucleic acids (RNA) and a deoxyribonucleic acids (DNA). In some embodiments, a therapeutic and/or prophylactic agent is selected from the group consisting of plasmid expression vectors, viral expression vectors, and mixtures thereof.

In some embodiments, the therapeutic and/or prophylactic agent is an agent that enhances (i.e., increases, stimulates, upregulates) protein expression. Non-limiting examples of types of therapeutic and/or prophylactic agents that can be used for enhancing protein expression include RNAs, mRNAs, dsRNAs, CRISPR/Cas9 technology, ssDNAs and DNAs (e.g., expression vectors). The agent that upregulates protein expression may upregulate expression of a naturally occurring or non-naturally occurring protein (e.g., a chimeric protein that has been modified to improve half life, or one that comprises desirable amino acid changes). Exemplary proteins include intracellular, transmembrane, or secreted proteins, peptides, or polypeptides.

In some embodiments, the therapeutic and/or prophylactic agent is a DNA therapeutic and/or prophylactic agent. For example, in some embodiments, when the therapeutic and/or prophylactic agent is a DNA, the DNA is selected from the group consisting of a double-stranded DNA, a single-stranded DNA (ssDNA), a partially double-stranded DNA i.e., has a portion that is double-stranded and a portion that is single-stranded, a triple stranded DNA, and a partially triple-stranded DNA, i.e., has a portion that is triple stranded and a portion that is double stranded. In some embodiments, the DNA is selected from the group consisting of a circular DNA, a linear DNA, and mixtures thereof.

A DNA therapeutic and/or prophylactic agent can be a DNA molecule that is capable of transferring a gene into a cell, e.g., that encodes and can express a transcript. In other embodiments, the DNA molecule is a synthetic molecule, e.g., a synthetic DNA molecule produced in vitro. In some embodiments, the DNA molecule is a recombinant molecule. Non-limiting exemplary DNA therapeutic and/or prophylactic agents include plasmid expression vectors and viral expression vectors.

The DNA therapeutic and/or prophylactic agents described herein, e.g., DNA vectors, can include a variety of different features. The DNA therapeutic and/or prophylactic agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

For example, in some embodiments, when the therapeutic and/or prophylactic agent is a RNA, the RNA is selected from the group consisting of a single-stranded RNA, a double-stranded RNA (dsRNA), a partially double-stranded RNA, and mixtures thereof. In some embodiments, the RNA is selected from the group consisting of a circular RNA, a linear RNA, and mixtures thereof.

For example, in some embodiments, when the therapeutic and/or prophylactic agent is a RNA, the RNA is selected from the group consisting of a short interfering RNA (siRNA), an asymmetrical interfering RNA (aiRNA), a RNA interference (RNAi) molecule, a microRNA (miRNA), an antagomir, an antisense RNA, a ribozyme, a Dicer-substrate RNA (dsRNA), a small hairpin RNA (shRNA), a messenger RNA (mRNA), locked nucleic acids (LNAs) and CRISPR/Cas9 technology, and mixtures thereof.

For example, in some embodiments, when the therapeutic and/or prophylactic agent is a RNA, the RNA is selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.

In some embodiments, the therapeutic and/or prophylactic agent is an mRNA. In some embodiments, the therapeutic and/or prophylactic agents is a modified mRNA (mmRNA).

In some embodiments, the therapeutic and/or prophylactic agent is an mRNA that incorporates a micro-RNA binding site (miR binding site). Further, in some embodiments, an mRNA includes one or more of a stem loop, a chain terminating nucleoside, a polyA sequence, a polyadenylation signal, and/or a 5′ cap structure.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. In some embodiments, all uracils or uridines are modified. When all nucleobases, nucleosides, or nucleotides are modified, e.g., all uracils or uridines, the mRNA can be referred to as “fully modified,” e.g., for uracil or uridine.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain terminating nucleotide into an mRNA, for example at the 3′-terminus, may result in stabilization of the mRNA.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A poly A sequence may also comprise stabilizing nucleotides or analogs. For example, a poly A sequence can include deoxythymidine, e.g., inverted (or reverse linkage) deoxythymidine (dT), as a stabilizing nucleotide or analog. Details on using inverted dT and other stabilizing poly A sequence modifications can be found, for example, in WO2017/049275 A2, the content of which is incorporated herein by reference. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site. MicroRNA binding sites (or miR binding sites) can be used to regulate mRNA expression in various tissues or cell types. In exemplary embodiments, miR binding sites are engineered into 3′ UTR sequences of an mRNA to regulate, e.g., enhance degradation of mRNA in cells or tissues expressing the cognate miR. Such regulation is useful to regulate or control “off-target” expression in mRNAs, i.e., expression in undesired cells or tissues in vivo. Details on using miR binding sites can be found, for example, in WO 2017/062513 A2, the content of which is incorporated herein by reference.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (m5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp3U), 1-methyl-3-(3-amino-3-carboxypropyl) pseudouridine (acp3 ψ), 5-(isopentenylaminomethyl) uridine (inm5U), 5-(isopentenylaminomethyl)-2-thio-uridine (inm5s2U), a-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include a-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms216A), N6-(cis-hydroxyisopentenyl) adenosine (io6A), 2-methylthio-N6-(cis-hydroxyisopentenyl) adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include a-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (02yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, a-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, 06-methyl-guanosine, 2′-F-ara-guanosine, and 2′-F-guanosine.

Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, the modified nucleobase is 1-methyl-pseudouridine (m1ψ), 5-methoxy-uridine (mo5U), 5-methyl-cytidine (m5C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is N1-methylpseudouridine (m1ψ) and the mRNA of the disclosure is fully modified with N1-methylpseudouridine (m1ψ). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In certain embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

The mmRNAs of the disclosure can include a combination of modifications to the sugar, the nucleobase, and/or the internucleoside linkage. These combinations can include any one or more modifications described herein.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, CA) and/or proprietary methods. In some embodiments, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In some embodiments, mRNAs are made using IVT enzymatic synthesis methods. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme.

Either enzymatic or chemical ligation methods may be used to conjugate polynucleotides or their regions with different functional moieties, such as targeting or delivery agents, fluorescent labels, liquids, nanoparticles, etc.

Therapeutic and/or Prophylactic Agents for Reducing Protein Expression

In some embodiments, the therapeutic and/or prophylactic agent is a therapeutic and/or prophylactic agent that reduces (i.e., decreases, inhibits, downregulates) protein expression. Non-limiting examples of types of therapeutic and/or prophylactic agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

Peptide/Polypeptide Therapeutic and/or Prophylactic Agents

In some embodiments, the therapeutic and/or prophylactic agent is a peptide therapeutic and/or prophylactic agent. In some embodiments the therapeutic and/or prophylactic agent is a polypeptide therapeutic and/or prophylactic agent.

In some embodiments, the peptide or polypeptide is naturally-derived, e.g., isolated from a natural source. In other embodiments, the peptide or polypeptide is a synthetic molecule, e.g., a synthetic peptide or polypeptide produced in vitro. In some embodiments, the peptide or polypeptide is a recombinant molecule. In some embodiments, the peptide or polypeptide is a chimeric molecule. In some embodiments, the peptide or polypeptide is a fusion molecule. In some embodiments, the peptide or polypeptide therapeutic and/or prophylactic agent of the composition is a naturally occurring peptide or polypeptide. In some embodiments, the peptide or polypeptide therapeutic and/or prophylactic agent in the composition is a modified version of a naturally occurring peptide or polypeptide (e.g., contains less than 3, less than 5, less than 10, less than 15, less than 20, or less than 25 amino substitutions, deletions, or additions compared to its wild type, naturally occurring peptide or polypeptide counterpart).

### Other LNP Components

An LNP may include one or more components in addition to those described in the preceding sections. For example, an LNP may include one or more small hydrophobic molecules such as a vitamin (e.g., vitamin A or vitamin E) or a sterol.

LNPs may also include one or more permeability enhancer molecules, carbohydrates, polymers, surface altering agents, or other components. Carbohydrates may include simple sugars (e.g., glucose) and polysaccharides (e.g., glycogen and derivatives and analogs thereof).

A polymer may be included in and/or used to encapsulate or partially encapsulate a nanoparticle composition. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl (meth)acrylate) (PMMA), poly(ethyl (meth)acrylate), poly(butyl (meth)acrylate), poly(isobutyl (meth)acrylate), poly(hexyl (meth)acrylate), poly(isodecyl (meth)acrylate), poly(lauryl (meth)acrylate), poly(phenyl (meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho) esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of an LNP (e.g., by coating, adsorption, covalent linkage, or other process).

AN LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of an LNP may also be conjugated with one or more useful antibodies. Functional groups and conjugates useful in targeted cell delivery, imaging, and membrane permeation are well known in the art.

In addition to these components, LNPs may include any substance useful in pharmaceutical compositions. For example, the LNP may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included.

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

A binding agent may be starch (e.g. cornstarch and starch paste); gelatin; sugars (e.g. sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Production of Nanoparticle Compositions

In some embodiments, nanoparticles (e.g., LNPs) comprising lipids of the disclosure are prepared by first combining the ionizable lipid (e.g., a compound of Formula (I), (I-a), (I-b), (I-c), (I-d), (I-e), or (I-f)), a phospholipid (e.g., DOPE or DSPC), a PEG lipid (e.g., PEG-DMG or PL-II (e.g., PEG-1)), and a structural lipid (e.g., cholesterol) in a buffer solution and then forming the nanoparticle, e.g., via nanoprecipitation. In some embodiments, nanoparticles of the disclosure are made according to methods described e.g., in International Patent Application Publication No. WO 2020/160397.

In certain embodiments, lipid stock solutions are mixed with mRNA stock solution (e.g., in a microfluidic mixer). The resulting LNPs can be buffer exchanged (e.g., by dialysis) into a storage buffer. The LNP solutions can then be concentrated and optionally purified by a sterile filter.

### Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a therapeutic and/or prophylactic agent (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of therapeutic and/or prophylactic agent in the nanoparticle composition can be calculated based on the extinction coefficient of the therapeutic and/or prophylactic agent used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, CA) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### In Vivo Formulation Studies

In order to monitor how effectively various nanoparticle compositions deliver therapeutic and/or prophylactic agents to targeted cells, different nanoparticle compositions including a particular therapeutic and/or prophylactic agent (for example, a modified or naturally occurring RNA such as an mRNA) are prepared and administered to animal populations. Animals (e.g., mice, rats, or non-human primates) are intravenously, intramuscularly, intraarterially, or intratumorally administered a single dose including a nanoparticle composition comprising a lipid of the disclosure and an mRNA expressing a protein, e.g., human erythropoietin (hEPO) or luciferase. A control composition including PBS may also be employed.

Upon administration of nanoparticle compositions to an animal, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the animals for evaluation may include blood, sera, and tissue (for example, muscle tissue from the site of an intramuscular injection and internal tissue); sample collection may involve sacrifice of the animals.

ELISA test kits may provide hEPO concentrations in milli International Units per mL (mIU/mL), e.g., per mL of tested serum. Typically, the EPO concentration of unknown sample is calculated from a calibration curve. Standards (used for the calibration curve) are calibrated according to the 2. IRP WHO preparation. In some embodiments, mIU/mL corresponds to 6.6 μg/mL. Nanoparticle compositions including mRNA are useful in the evaluation of the efficacy and usefulness of various formulations for the delivery of therapeutic and/or prophylactic agents. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the therapeutic and/or prophylactic agent by a given nanoparticle composition relative to other nanoparticle compositions or the absence thereof.

In some embodiments, an in vivo expression assay may be used to assess potency of expression of lipids of the disclosure. In some embodiments, the protein expression (hEPO) may be measured in mice following administration of a nanoparticle comprising a lipid of the disclosure (e.g., a loaded LNP). In some embodiments, lipid nanoparticles of the instant disclosure may be intravenously administered to mice (e.g., CD-1 mice).

In some embodiments, lipid nanoparticles (LNPs) may include DSPC as a phospholipid, cholesterol as a structural lipid, PL-II (e.g., PEG-1) as a PEG lipid, an ionizable lipid (e.g., a compound of Formula (I), (I-a), (I-b), (I-c), (I-d), (I-e), or (I-f)), and an mRNA encoding hEPO. In some embodiments, lipid nanoparticles (LNPs) may include DSPC as a phospholipid, cholesterol as a structural lipid, PL-III (e.g., PEG_{2K}DMG) as a PEG lipid, an ionizable lipid (e.g., a compound of Formula (I), (I-a), (I-b), (I-c), (I-d), (I-e), or (I-f)), and an mRNA encoding hEPO.

In some embodiments, the concentration of hEPO in serum may be tested after administration (e.g., about six hours after injection). In some embodiments, residual levels of the lipids of the disclosure in organs or tissue of the subject after administration (e.g., 6 h, 12 h, 18 h, 24 h, 36h, or 48 h after administration) are measured. In some embodiments, the residual levels of the lipids of the disclosure in the liver are measured. In some embodiments, an in vitro expression assay may be used to assess nanoparticles of the disclosure. In some embodiments, cells (e.g., HeLa) may be plated in an imaging plate (e.g., poly-D-lysine coated) and cultured in serum (e.g., human serum, mouse serum, cynomolgus monkey serum or fetal bovine serum). In some embodiments, lipid nanoparticles of the disclosure comprising an mRNA expressing fluorescent protein (e.g., green fluorescent protein (GFP)) and a fluorescent lipid (e.g., rhodamine-DOPE) may be added to the plate and the plate imaged for uptake and expression. In some embodiments, expression may be evaluated by measuring fluorescence (e.g., from GFP). In some embodiments, uptake (accumulation) may be evaluated by measuring the fluorescence signal from a fluorescent lipid (e.g., rhodamine-DOPE).

### LNP Formulations

LNPs may include a lipid component and one or more additional components, such as a therapeutic and/or prophylactic agent. An LNP may be designed for one or more specific applications or targets. The elements of an LNP may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, an ionizable lipid (e.g., a compound of Formula (I), (I-a), (I-b), (I-c), (I-d), (I-e), or (I-f)), a phospholipid (such as an unsaturated lipid, e.g., DOPE or DSPC), a PEG lipid, and a structural lipid. The elements of the lipid component may be provided in specific fractions.

In some embodiments, the lipid component of a nanoparticle composition includes an ionizable lipid (e.g., a compound of Formula (I), (I-a), (I-b), (I-c), (I-d), (I-e), or (I-f)), a phospholipid, a PEG lipid, and a structural lipid. In certain embodiments, the lipid component of the nanoparticle composition includes about 30 mol % to about 60 mol % ionizable lipid (e.g., a compound of Formula (I), (I-a), (I-b), (I-c), (I-d), (I-e), or (I-f)), about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % ionizable lipid (e.g., a compound of Formula (I), (I-a), (I-b), (I-c), (I-d), (I-e), or (I-f)), about 5 mol % to about 25 mol % phospholipid, about 30 mol % to about 40 mol % structural lipid, and about 0 mol % to about 10 mol % of PEG lipid. In a particular embodiment, the lipid component includes about 50 mol % ionizable lipid (e.g., a compound of Formula (I), (I-a), (I-b), (I-c), (I-d), (I-e), or (I-f)), about 10 mol % phospholipid, about 38.5 mol % structural lipid, and about 1.5 mol % of PEG lipid. In another particular embodiment, the lipid component includes about 40 mol % ionizable lipid (e.g., a compound of Formula (I), (I-a), (I-b), (I-c), (I-d), (I-e), or (I-f)), about 20 mol % phospholipid, about 38.5 mol % structural lipid, and about 1.5 mol % of PEG lipid. In some embodiments, the phospholipid may be DOPE or DSPC. In other embodiments, the PEG lipid may be PL-II (e.g., PEG-1), or PL-III (e.g., PEG₂ₖ-DMG) and/or the structural lipid may be cholesterol.

In some embodiments an empty lipid nanoparticle (empty LNP) comprises an ionizable lipid (e.g., a compound of Formula (I), (I-a), (I-b), (I-c), (I-d), (I-e), or (I-f)), a phospholipid, a structural lipid, and a PEG lipid. In some embodiments a loaded lipid nanoparticle (loaded LNP) comprises an ionizable lipid (e.g., a compound of Formula (I), (I-a), (I-b), (I-c), (I-d), (I-e), or (I-f)), a phospholipid, a structural lipid, a PEG lipid, and one or more therapeutic and/or prophylactic agents.

In some embodiments, the LNP comprises the ionizable lipid (e.g., a compound of Formula (I), (I-a), (I-b), (I-c), (I-d), (I-e), or (I-f)), in an amount from about 40% to about 60%.

In some embodiments, the LNP comprises the phospholipid in an amount from about 0% to about 20%. For example, in some embodiments, the LNP comprises DSPC in an amount from about 0% to about 20%.

In some embodiments, the LNP comprises the structural lipid in an amount from about 30% to about 50%. For example, in some embodiments, the LNP comprises cholesterol in an amount from about 30% to about 50%.

In some embodiments, the LNP comprises the PEG lipid in an amount from about 0% to about 5%. For example, in some embodiments, the LNP comprises PL-II (e.g., PEG-1) or PEG₂ₖ-DMG in an amount from about 0% to about 5%.

In some embodiments, the LNP comprises about 40 mol % to about 60 mol % of the ionizable lipid (e.g., a compound of Formula (I), (I-a), (I-b), (I-c), (I-d), (I-e), or (I-f)), about 0 mol % to about 20 mol % phospholipid, about 30 mol % to about 50 mol % structural lipid, and about 0 mol % to about 5 mol % PEG lipid. In some embodiments, the LNP comprises about 40 mol % to about 60 mol % of the ionizable lipid (e.g., a compound of Formula (I), (I-a), (I-b), (I-c), (I-d), (I-e), or (I-f)), about 0 mol % to about 20 mol % DSPC, about 30 mol % to about 50 mol % cholesterol, and about 0 mol % to about 5 mol % PL-III (e.g., PEG₂ₖ-DMG). In some embodiments, the LNP comprises about 40 mol % to about 60 mol % of the lipid of Table 1, about 0 mol % to about 20 mol % DSPC, about 30 mol % to about 50 mol % cholesterol, and about 0 mol % to about 5 mol % PL-III (e.g., PEG₂ₖ-DMG). In some embodiments, the LNP comprises about 40 mol % to about 60 mol % of the ionizable lipid (e.g., a compound of Formula (I), (I-a), (I-b), (I-c), (I-d), (I-e), or (I-f)), about 0 mol % to about 20 mol % DSPC, about 30 mol % to about 50 mol % cholesterol, and about 0 mol % to about 5 mol % PL-II (e.g., PEG-1). In some embodiments, the LNP comprises about 40 mol % to about 60 mol % of the lipid of Table 1, about 0 mol % to about 20 mol % DSPC, about 30 mol % to about 50 mol % cholesterol, and about 0 mol % to about 5 mol % PL-II (e.g., PEG-1).

In some embodiments, the LNP comprises an ionizable lipid (e.g., a compound of Formula (I), (I-a), (I-b), (I-c), (I-d), (I-e), or (I-f)), a phospholipid, a structural lipid, and a PEG lipid, wherein the phospholipid is DSPC and the structural lipid is cholesterol. In some embodiments, the LNP comprises a lipid of Table 1, a phospholipid, a structural lipid, and a PEG lipid, wherein the phospholipid is DSPC and the structural lipid is cholesterol.

In some embodiments, the LNP comprises an ionizable lipid (e.g., a compound of Formula (I), (I-a), (I-b), (I-c), (I-d), (I-e), or (I-f)), a phospholipid, a structural lipid, and a PEG lipid, wherein the structural lipid is cholesterol and the PEG lipid is PL-III (e.g., PEG₂ₖ-DMG). In some embodiments, the LNP comprises a lipid of Table 1, a phospholipid, a structural lipid, and a PEG lipid, wherein the structural lipid is cholesterol and the PEG lipid is PL-III (e.g., PEG₂ₖ-DMG). In some embodiments, the LNP comprises an ionizable lipid (e.g., a compound of Formula (I), (I-a), (I-b), (I-c), (I-d), (I-e), or (I-f)), a phospholipid, a structural lipid, and a PEG lipid, wherein the structural lipid is cholesterol and the PEG lipid is PL-II (e.g., PEG-1). In some embodiments, the LNP comprises a lipid of Table 1, a phospholipid, a structural lipid, and a PEG lipid, wherein the structural lipid is cholesterol and the PEG lipid is PL-II (e.g., PEG-1).

In some embodiments, the LNP comprises an ionizable lipid (e.g., a compound of Formula (I), (I-a), (I-b), (I-c), (I-d), (I-e), or (I-f)), a phospholipid, a structural lipid, and a PEG lipid, wherein the phospholipid is DSPC and the PEG lipid is PL-III (e.g., PEG₂ₖ-DMG). In some embodiments, the LNP comprises a lipid of Table 1, a phospholipid, a structural lipid, and a PEG lipid, wherein the phospholipid is DSPC and the PEG lipid is PL-III (e.g., PEG₂ₖ-DMG). In some embodiments, the LNP comprises an ionizable lipid (e.g., a compound of Formula (I), (I-a), (I-b), (I-c), (I-d), (I-e), or (I-f)), a phospholipid, a structural lipid, and a PEG lipid, wherein the phospholipid is DSPC and the PEG lipid is PL-II (e.g., PEG-1). In some embodiments, the LNP comprises a lipid of Table 1, a phospholipid, a structural lipid, and a PEG lipid, wherein the phospholipid is DSPC and the PEG lipid is PL-II (e.g., PEG-1).

In some embodiments, the LNP comprises an ionizable lipid (e.g., a compound of Formula (I), (I-a), (I-b), (I-c), (I-d), (I-e), or (I-f)), a phospholipid, a structural lipid, and a PEG lipid, wherein the phospholipid is DSPC, the structural lipid is cholesterol, and the PEG lipid is PL-III (e.g., PEG₂ₖ-DMG). In some embodiments, the LNP comprises a lipid of Table 1, a phospholipid, a structural lipid, and a PEG lipid, wherein the phospholipid is DSPC, the structural lipid is cholesterol, and the PEG lipid is PL-III (e.g., PEG₂ₖ-DMG). In some embodiments, the LNP comprises a lipid of Table 1, a phospholipid, a structural lipid, and a PEG lipid, wherein the phospholipid is DSPC, the structural lipid is cholesterol, and the PEG lipid is PL-III (e.g., PEG₂ₖ-DMG). In some embodiments, the LNP comprises an ionizable lipid (e.g., a compound of Formula (I), (I-a), (I-b), (I-c), (I-d), (I-e), or (I-f)), a phospholipid, a structural lipid, and a PEG lipid, wherein the phospholipid is DSPC, the structural lipid is cholesterol, and the PEG lipid is PL-II (e.g., PEG-1). In some embodiments, the LNP comprises a lipid of Table 1, a phospholipid, a structural lipid, and a PEG lipid, wherein the phospholipid is DSPC, the structural lipid is cholesterol, and the PEG lipid is PL-II (e.g., PEG-1).

LNPs may be designed for one or more specific applications or targets. For example, a nanoparticle composition may be designed to deliver a therapeutic and/or prophylactic agent such as an RNA to a particular cell, tissue, organ, or system or group thereof in a mammal's body. Physiochemical properties of LNPs may be altered in order to increase selectivity for particular bodily targets. For instance, particle sizes may be adjusted based on the fenestration sizes of different organs. The therapeutic and/or prophylactic agents included in a nanoparticle composition may also be selected based on the desired delivery target or targets. For example, a therapeutic and/or prophylactic agents may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver.

The amount of a therapeutic and/or prophylactic agent in a nanoparticle composition may depend on the size, composition, desired target and/or application, or other properties of the nanoparticle composition as well as on the properties of the therapeutic and/or prophylactic agent. For example, the amount of an RNA useful in a nanoparticle composition may depend on the size, sequence, and other characteristics of the RNA. The relative amounts of a therapeutic and/or prophylactic agent and other elements (e.g., lipids) in a nanoparticle composition may also vary. In some embodiments, the wt/wt ratio of the lipid component to a therapeutic and/or prophylactic agent in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic agent may be from about 10:1 to about 40:1. In certain embodiments, the wt/wt ratio is about 20:1.

The amount of a therapeutic and/or prophylactic agent in a nanoparticle composition may, for example, be measured using absorption spectroscopy (e.g., ultraviolet-visible spectroscopy).

In some embodiments, a nanoparticle composition includes one or more RNAs, and the one or more RNAs, lipids, and amounts thereof may be selected to provide a specific N: P ratio. The N: P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N: P ratio may be from about 2:1 to about 8:1. In other embodiments, the N: P ratio is from about 5:1 to about 8:1. For example, the N: P ratio may be about 5.0:1, about 5.5:1, about 5.67:1, about 6.0:1, about 6.5:1, or about 7.0:1. For example, the N: P ratio may be about 5.67:1.

### Physical Properties of LNPs

The characteristics of an LNP may depend on the components thereof. For example, an LNP including cholesterol as a structural lipid may have different characteristics than an LNP that includes a different structural lipid. Similarly, the characteristics of an LNP may depend on the absolute or relative amounts of its components. For instance, an LNP including a higher molar fraction of a phospholipid may have different characteristics than an LNP including a lower molar fraction of a phospholipid. Characteristics may also vary depending on the method and conditions of preparation of the nanoparticle composition.

LNPs may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

In some embodiments, the mean diameter of a lipid nanoparticle of the disclosure is between 10s of nm and 100s of nm as measured by dynamic light scattering (DLS). For example, in some embodiments, the mean diameter a lipid nanoparticle of the disclosure is from about 40 nm to about 150 nm. In some embodiments, the mean diameter a lipid nanoparticle of the disclosure is about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean diameter of an LNP is from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 150 nm, from about 70 nm to about 130 nm, from about 70 nm to about 100 nm, from about 70 nm to about 90 nm, from about 70 nm to about 80 nm, from about 80 nm to about 150 nm, from about 80 nm to about 130 nm, from about 80 nm to about 100 nm, from about 80 nm to about 90 nm, from about 90 nm to about 150 nm, from about 90 nm to about 130 nm, or from about 90 nm to about 100 nm. In certain embodiments, the mean diameter of an LNP of the disclosure is from about 70 nm to about 130 nm or from about 70 nm to about 100 nm. In some embodiments, the mean diameter of a nanoparticle of the disclosure is about 80 nm. In some embodiments, the mean diameter of a nanoparticle of the disclosure is about 100 nm. In some embodiments, the mean diameter of a nanoparticle of the disclosure is about 110 nm. In some embodiments, the mean diameter of a nanoparticle of the disclosure is about 120 nm.

In some embodiments, the polydispersity index (“PDI”) of a plurality of LNPs formulated with lipids of the disclosure is less than 0.3. In some embodiments, plurality of LNPs formulated with lipids of the disclosure has a polydispersity index of from about 0 to about 0.25. In some embodiments, plurality of LNPs formulated with lipids of the disclosure has a polydispersity index of from about 0.10 to about 0.20.

Surface hydrophobicity of nanoparticles of the disclosure can be measured by Generalized Polarization by Laurdan (GPL). In this method, Laurdan, a fluorescent aminonaphthalene ketone lipid, is post-inserted into the nanoparticle surface and the fluorescence spectrum of Laurdan is collected to determine the normalized Generalized Polarization (N-GP). In some embodiments, nanoparticles formulated with lipids of the disclosure have a surface hydrophobicity expressed as N-GP of between about 0.5 and about 1.5. For example, in some embodiments, nanoparticles formulated with lipids of the disclosure have a surface hydrophobicity expressed as N-GP of about 0.5, about 0.6, about 0.7, about 0.8, about 0.9, about 1.0, about 1.1, about 1.2, about 1.3, about 1.4, or about 1.5. In some embodiments, nanoparticles formulated with lipids of the disclosure have a surface hydrophobicity expressed as N-GP of about 1.0 or about 1.1.

The zeta potential of an LNP may be used to indicate the electrokinetic potential of the composition. For example, the zeta potential may describe the surface charge of a nanoparticle composition. LNPs with relatively low charges, positive or negative, are generally desirable, as more highly charged species may interact undesirably with cells, tissues, and other elements in the body. In some embodiments, the zeta potential of an LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic agent describes the amount of therapeutic and/or prophylactic agent that is encapsulated or otherwise associated with an LNP after preparation, relative to the initial amount provided. The encapsulation efficiency is desired to be high (e.g., close to 100%). The encapsulation efficiency may be measured, for example, by comparing the amount of therapeutic and/or prophylactic agent in a solution containing a loaded LNP before and after breaking up the loaded LNP with one or more organic solvents or detergents. Fluorescence may be used to measure the amount of free therapeutic and/or prophylactic agent (e.g., RNA) in a solution. For the loaded LNPs formulated with lipids of the disclosure, the encapsulation efficiency of a therapeutic and/or prophylactic agent is at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency is at least 80%. In some embodiments, the encapsulation efficiency is at least 90%. In some embodiments, the encapsulation efficiency of the therapeutic and/or prophylactic agent is between 80% and 100%.

### Pharmaceutical Compositions

LNPs may be formulated in whole or in part as pharmaceutical compositions. Pharmaceutical compositions may include one or more LNPs. In one embodiment, a pharmaceutical composition comprises a population of LNPs. For example, a pharmaceutical composition may include one or more LNPs including one or more different therapeutic and/or prophylactic agents. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a nanoparticle composition. An excipient or accessory ingredient may be incompatible with a component of an LNP if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more LNPs, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more LNPs.

In certain embodiments, the LNPs and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., —130° C. or −150° C.). For example, the pharmaceutical composition comprising an ionizable lipid (e.g., a compound of Formula (I), (I-a), (I-b), (I-c), (I-d), (I-e), or (I-f)) is a solution that is refrigerated for storage and/or shipment at, for example, about −20° C., −30° C., −40° C., −50° C., −60° C., −70° C., or −80° C. In certain embodiments, the disclosure also relates to a method of increasing stability of the LNPs and/or pharmaceutical compositions comprising an ionizable lipid (e.g., a compound of Formula (I), (I-a), (I-b), (I-c), (I-d), (I-e), or (I-f)) by storing the LNPs and/or pharmaceutical compositions at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., —90° C., —130° C. or −150° C.). For example, the LNPs and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.). In some embodiments, the formulation is stabilized for at least 4 weeks at about 4° C. In certain embodiments, the pharmaceutical composition of the disclosure comprises an LNP disclosed herein and a pharmaceutically acceptable carrier selected from one or more of Tris, an acetate (e.g., sodium acetate), an citrate (e.g., sodium citrate), saline, PBS, and sucrose. In certain embodiments, the pharmaceutical composition of the disclosure has a pH value between about 7 and 8 (e.g., 6.8 6.9, 7.0, 7.1, 7.2, 7.3, 7.4, 7.5, 7.6, 7.7, 7.8, 7.9 or 8.0, or between 7.5 and 8 or between 7 and 7.8). For example, a pharmaceutical composition of the disclosure comprises an LNP disclosed herein, Tris, saline and sucrose, and has a pH of about 7.5-8, which is suitable for storage and/or shipment at, for example, about −20° C. For example, a pharmaceutical composition of the disclosure comprises an LNP disclosed herein and PBS and has a pH of about 7-7.8, suitable for storage and/or shipment at, for example, about 4° C. or lower.

“Stability,” “stabilized,” and “stable” in the context of the present disclosure refers to the resistance of LNPs and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

In some embodiments, a pharmaceutical composition of the disclosure comprises a empty LNP or a loaded LNP, a cryoprotectant, a buffer, or a combination thereof.

In some embodiments, the cryoprotectant comprises one or more cryoprotective agents, and each of the one or more cryoprotective agents is independently a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG₂ₖ-DMG, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium acetate, sodium chloride, sodium formate, sodium malonate, sodium nitrate, sodium sulfate, or any hydrate thereof), or any combination thereof. In some embodiments, the cryoprotectant comprises sucrose. In some embodiments, the cryoprotectant and/or excipient is sucrose. In some embodiments, the cryoprotectant comprises sodium acetate. In some embodiments, the cryoprotectant and/or excipient is sodium acetate. In some embodiments, the cryoprotectant comprises sucrose and sodium acetate.

In some embodiments, the buffer is selected from the group consisting of an acetate buffer, a citrate buffer, a phosphate buffer, a tris buffer, and combinations thereof.

LNPs and/or pharmaceutical compositions including one or more LNPs may be administered to any patient or subject, including those patients or subjects that may benefit from a therapeutic effect provided by the delivery of a therapeutic and/or prophylactic agent to one or more particular cells, tissues, organs, or systems or groups thereof. Although the descriptions provided herein of LNPs and pharmaceutical compositions including LNPs are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, horses, sheep, cats, dogs, mice, and/or rats. The subject lipid nanoparticles can also be employed for in vitro and ex vivo uses.

A pharmaceutical composition including one or more LNPs may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutic and/or prophylactic agents, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay, silicates), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices. Intradermal compositions may be administered by devices which limit the effective penetration length of a needle into the skin. Jet injection devices which deliver liquid compositions to the dermis via a liquid jet injector and/or via a needle which pierces the stratum corneum and produces a jet which reaches the dermis are suitable. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e., by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

mRNA Therapies

mRNA as a drug modality has the potential to deliver secreted proteins as well as intracellular proteins and transmembrane proteins. mRNA as a drug modality has the potential to deliver transmembrane and intracellular proteins, i.e., targets that standard biologics are unable to access owing to their inability to cross the cell membrane when delivered in protein form. One major challenge to making mRNA based therapies a reality is the identification of an optimal delivery vehicle. Due to its large size, chemical instability and potential immunogenicity, mRNA requires a delivery vehicle that can offer protection from endo- and exo-nucleases, as well as shield the cargo from immune sentinels. Lipid nanoparticles (LNPs) have been identified as a leading option in this regard.

Key performance criteria for a lipid nanoparticle delivery system are to maximize cellular uptake and enable efficient release of mRNA from the endosome. In one embodiment, the subject LNPs comprising the novel lipids disclosed herein, demonstrate improvements in at least one of cellular uptake and endosomal release. At the same time the LNP must provide a stable drug product and be able to be dosed safely at therapeutically relevant levels. LNPs are multi-component systems which typically consist of an amino lipid, phospholipid, cholesterol, and a PEG-lipid. Each component is required for aspects of efficient delivery of the nucleic acid cargo and stability of the particle. The key component thought to drive cellular uptake, endosomal escape, and tolerability is the amino lipid. Cholesterol and the PEG-lipid contribute to the stability of the drug product both in vivo and on the shelf, while the phospholipid provides additional fusogenicity to the LNP, thus helping to drive endosomal escape and rendering the nucleic acid bioavailable in the cytosol of cells.

Several amino lipid series have been developed for oligonucleotide delivery over the past couple of decades, including the amino lipid MC3 (DLin-MC3-DMA). MC3-based LNPs have been shown to be effective in delivering mRNA. LNPs of this class are quickly opsonized by apolipoprotein E (ApoE) when delivered intravenously, which enables cellular uptake by the low density lipoprotein receptor (LDLr). However, concerns remain that MC3's long tissue half-life could contribute to unfavorable side effects hindering its use for chronic therapies. In addition, extensive literature evidence suggests that chronic dosing of lipid nanoparticles can produce several toxic sides effects including complement activation-related pseudo allergy (CARPA) and liver damage. Hence, to unleash the potential of mRNA and other nucleic acid, nucleoptide or peptide based therapies for humans, a class of LNPs with increased delivery efficiency along with a metabolic and toxicity profile that would enable chronic dosing in humans is needed.

The ability to treat a broad swath of diseases requires the flexibility to safely dose chronically at varying dose levels. Through systematic optimization of the amino lipid structure, the lipids of the disclosure were identified as lipids that balance chemical stability, improved efficiency of delivery due to improved endosomal escape, rapid in vivo metabolism, and a clean toxicity profile. The combination of these features provides a drug candidate that can be dosed chronically without activation of the immune system. Initial rodent screens led to the identification of a lead lipid with good delivery efficiency and pharmacokinetics. The lead LNP was profiled further in non-human primate for efficiency of delivery after single and repeat dosing. Finally, the optimized LNPs were evaluated in one-month repeat dose toxicity studies in rat and non-human primate. Without wishing to be bound by theory, the novel ionizable lipids of the instant disclosure have the improved cellular delivery, improved protein expression, and improved biodegradability properties that can lead to greater than 2 fold, 5 fold, 10 fold, 15 fold, or 20 fold increase in mRNA expression in cells as compared to LNPs which lack a lipid of the invention. In another embodiment, an LNP comprising a lipid of the invention can result in specific (e.g., preferential) delivery to a certain cell type or types as compared other cell types, thereby resulting in a greater than 2 fold, 5 fold, 10 fold, 15 fold, or 20 fold increase in mRNA expression in certain cells or tissues as compared to LNPs which lack a lipid of the invention. These improvements over the art allow for the safe and effective use of mRNA-based therapies in acute and chronic diseases.

### Methods of Treatment and Uses

In some aspects, the disclosure provides a method of delivering a therapeutic and/or prophylactic agent to a cell (e.g., a mammalian cell). This method includes the step of contacting the cell with a loaded LNP or a pharmaceutical composition of the disclosure, whereby the therapeutic and/or prophylactic agent is delivered to the cell. In some embodiments, the cell is in a subject and the contacting comprises administering the cell to the subject. In some embodiments, the method comprises the step of administering to the subject a lipid nanoparticle comprising an ionizable lipid (e.g., a compound of Formula (I), (I-a), (I-b), (I-c), (I-d), (I-e), or (I-f)), a phospholipid, a structural lipid, a PEG lipid, and one or more therapeutic and/or prophylactic agents, whereby the therapeutic and/or prophylactic agent is delivered to the cell.

In some embodiments, the disclosure provides a method of delivering a therapeutic and/or prophylactic agent to a cell within a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-III (e.g., PEG₂ₖ-DMG), and one or more therapeutic and/or prophylactic agents selected from a nucleotide, a polypeptide, and a nucleic acid (e.g., an RNA). In some embodiments, the disclosure provides a method of delivering a therapeutic and/or prophylactic agent to a cell within a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-II (e.g., PEG-1), and one or more therapeutic and/or prophylactic agents selected from a nucleotide, a polypeptide, and a nucleic acid (e.g., an RNA).

In some aspects, the disclosure provides a method of delivering (e.g., specifically delivering) a therapeutic and/or prophylactic agent to a mammalian organ or tissue (e.g., a liver, kidney, spleen, or lung). This method includes the step of contacting the organ or tissue with a loaded LNP or a pharmaceutical composition of the disclosure, whereby the therapeutic and/or prophylactic agent is delivered to the target organ or tissue. In some embodiments, the method comprises the step of administering to the subject a lipid nanoparticle comprising an ionizable lipid (e.g., a compound of Formula (I), (I-a), (I-b), (I-c), (I-d), (I-e), or (I-f)), a phospholipid, a structural lipid, a PEG lipid, and one or more therapeutic and/or prophylactic agents, whereby the therapeutic and/or prophylactic agent is delivered to the target organ or tissue.

In some embodiments, the disclosure provides a method of specifically delivering a therapeutic and/or prophylactic agent to an organ of a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-III (e.g., PEG₂ₖ-DMG), and one or more therapeutic and/or prophylactic agents selected from a nucleotide, a polypeptide, and a nucleic acid (e.g., an RNA). In some embodiments, the disclosure provides a method of specifically delivering a therapeutic and/or prophylactic agent to an organ of a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-II (e.g., PEG-1), and one or more therapeutic and/or prophylactic agents selected from a nucleotide, a polypeptide, and a nucleic acid (e.g., an RNA).

In some aspects, the disclosure features a method for the enhanced delivery of a therapeutic and/or prophylactic agent (e.g., an mRNA) to a target tissue (e.g., a liver, spleen, or lung). This method includes the step of contacting the cell with a loaded LNP or a pharmaceutical composition of the disclosure, whereby the therapeutic and/or prophylactic agent is delivered to the target tissue (e.g., a liver, kidney, spleen, or lung). In some embodiments, the method comprises the step of administering to the subject a lipid nanoparticle comprising an ionizable lipid (e.g., a compound of Formula (I), (I-a), (I-b), (I-c), (I-d), (I-e), or (I-f)), a phospholipid, a structural lipid, a PEG lipid, and one or more therapeutic and/or prophylactic agents, whereby the therapeutic and/or prophylactic agent is delivered to the target tissue (e.g., a liver, kidney, spleen, or lung).

In some embodiments, the disclosure provides a method for the enhanced delivery of a therapeutic and/or prophylactic agent to a target tissue, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-III (e.g., PEG₂ₖ-DMG), and one or more therapeutic and/or prophylactic agents selected from a nucleotide, a polypeptide, and a nucleic acid (e.g., an RNA). In some embodiments, the disclosure provides a method for the enhanced delivery of a therapeutic and/or prophylactic agent to a target tissue, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-II (e.g., PEG-1), and one or more therapeutic and/or prophylactic agents selected from a nucleotide, a polypeptide, and a nucleic acid (e.g., an RNA).

In some aspects, the disclosure provides a method of producing a polypeptide of interest in a cell (e.g., a mammalian cell). This method includes the step of contacting the cell with a loaded LNP or a pharmaceutical composition of the disclosure, wherein the loaded LNP or pharmaceutical composition comprises an mRNA, whereby the mRNA is capable of being translated in the cell to produce the polypeptide. In some embodiments, the cell is in a subject and the contacting comprises administering the cell to the subject. In some embodiments, the method comprises the step of administering to the subject a lipid nanoparticle comprising an ionizable lipid (e.g., a compound of Formula (I), (I-a), (I-b), (I-c), (I-d), (I-e), or (I-f)), a phospholipid, a structural lipid, a PEG lipid, and an mRNA, whereby the mRNA is capable of being translated in the cell to produce the polypeptide.

In some embodiments, the disclosure provides a method of producing a polypeptide of interest in a cell, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-III (e.g., PEG₂ₖ-DMG), and an mRNA. For example, in some embodiments, the disclosure provides a method of producing a polypeptide of interest in a cell, wherein the method comprises the step of administering to the subject a lipid nanoparticle comprising a lipid of Table 1, DSPC, cholesterol, and PL-III (e.g., PEG₂ₖ-DMG), and an mRNA. In some embodiments, the disclosure provides a method of producing a polypeptide of interest in a cell, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-II (e.g., PEG-1), and an mRNA. For example, in some embodiments, the disclosure provides a method of producing a polypeptide of interest in a cell, wherein the method comprises the step of administering to the subject a lipid nanoparticle comprising a lipid of Table 1, DSPC, cholesterol, and PL-II (e.g., PEG-1), and an mRNA.

In some aspects, the disclosure provides a method of treating or preventing a disease or disorder in a mammal (e.g., a human) in need thereof. The method includes the step of administering to the mammal the loaded LNP or a pharmaceutical composition of the disclosure. In some aspects, the disclosure provides a method of treating a disease or disorder in a mammal (e.g., a human) in need thereof. The method includes the step of administering to the mammal the loaded LNP or a pharmaceutical composition of the disclosure. In some aspects, the disclosure provides a method of preventing a disease or disorder in a mammal (e.g., a human) in need thereof. The method includes the step of administering to the mammal the loaded LNP or a pharmaceutical composition of the disclosure. In some embodiments, the method comprises the step of administering to the subject a lipid nanoparticle comprising an ionizable lipid (e.g., a compound of Formula (I), (I-a), (I-b), (I-c), (I-d), (I-e), or (I-f)), a phospholipid, a structural lipid, a PEG lipid, and one or more therapeutic and/or prophylactic agents, whereby the therapeutic and/or prophylactic agent is delivered to the cell. In some embodiments, the disease or disorder is characterized by dysfunctional or aberrant protein or polypeptide activity. For example, the disease or disorder is selected from the group consisting of rare diseases, infectious diseases, cancer and proliferative diseases, genetic diseases, autoimmune diseases, diabetes, neurodegenerative diseases, cardio- and reno-vascular diseases, and metabolic diseases.

In some embodiments, the disclosure provides a method of treating or preventing a disease or disorder in a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-III (e.g., PEG₂ₖ-DMG), and one or more therapeutic and/or prophylactic agents selected from a nucleotide, a polypeptide, and a nucleic acid (e.g., an RNA). In some embodiments, the disclosure provides a method of treating a disease or disorder in a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-III (e.g., PEG₂ₖ-DMG), and one or more therapeutic and/or prophylactic agents selected from a nucleotide, a polypeptide, and a nucleic acid (e.g., an RNA). In some embodiments, the disclosure provides a method of preventing a disease or disorder in a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-III (e.g., PEG₂ₖ-DMG), and one or more therapeutic and/or prophylactic agents selected from a nucleotide, a polypeptide, and a nucleic acid (e.g., an RNA).

For example, in some embodiments, the disclosure provides a method of treating or preventing a disease or disorder in a subject, wherein the method comprises the step of administering to the subject a lipid nanoparticle comprising a lipid of Table 1, DSPC, cholesterol, and PL-III (e.g., PEG₂ₖ-DMG), and one or more therapeutic and/or prophylactic agents selected from a nucleotide, a polypeptide, and a nucleic acid (e.g., an RNA). For example, in some embodiments, the disclosure provides a method of treating a disease or disorder in a subject, wherein the method comprises the step of administering to the subject a lipid nanoparticle comprising a lipid of Table 1, DSPC, cholesterol, and PL-III (e.g., PEG₂ₖ-DMG), and one or more therapeutic and/or prophylactic agents selected from a nucleotide, a polypeptide, and a nucleic acid (e.g., an RNA). For example, in some embodiments, the disclosure provides a method of preventing a disease or disorder in a subject, wherein the method comprises the step of administering to the subject a lipid nanoparticle comprising a lipid of Table 1, DSPC, cholesterol, and PL-III (e.g., PEG₂ₖ-DMG), and one or more therapeutic and/or prophylactic agents selected from a nucleotide, a polypeptide, and a nucleic acid (e.g., an RNA).

In some embodiments, the disclosure provides a method of treating or preventing a disease or disorder in a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-II (e.g., PEG-1), and one or more therapeutic and/or prophylactic agents selected from a nucleotide, a polypeptide, and a nucleic acid (e.g., an RNA). In some embodiments, the disclosure provides a method of treating a disease or disorder in a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-II (e.g., PEG-1), and one or more therapeutic and/or prophylactic agents selected from a nucleotide, a polypeptide, and a nucleic acid (e.g., an RNA). In some embodiments, the disclosure provides a method of preventing a disease or disorder in a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-II (e.g., PEG-1), and one or more therapeutic and/or prophylactic agents selected from a nucleotide, a polypeptide, and a nucleic acid (e.g., an RNA).

For example, in some embodiments, the disclosure provides a method of treating or preventing a disease or disorder in a subject, wherein the method comprises the step of administering to the subject a lipid nanoparticle comprising a lipid of Table 1, DSPC, cholesterol, and PL-II (e.g., PEG-1), and one or more therapeutic and/or prophylactic agents selected from a nucleotide, a polypeptide, and a nucleic acid (e.g., an RNA). For example, in some embodiments, the disclosure provides a method of treating a disease or disorder in a subject, wherein the method comprises the step of administering to the subject a lipid nanoparticle comprising a lipid of Table 1, DSPC, cholesterol, and PL-II (e.g., PEG-1), and one or more therapeutic and/or prophylactic agents selected from a nucleotide, a polypeptide, and a nucleic acid (e.g., an RNA). For example, in some embodiments, the disclosure provides a method of preventing a disease or disorder in a subject, wherein the method comprises the step of administering to the subject a lipid nanoparticle comprising a lipid of Table 1, DSPC, cholesterol, and PL-II (e.g., PEG-1), and one or more therapeutic and/or prophylactic agents selected from a nucleotide, a polypeptide, and a nucleic acid (e.g., an RNA).

In yet another aspect, the disclosure features a method of lowering immunogenicity comprising introducing loaded LNP or a pharmaceutical composition of the disclosure into cells, wherein the loaded LNP or a pharmaceutical composition reduces the induction of the cellular immune response of the cells to the loaded LNP or a pharmaceutical composition, as compared to the induction of the cellular immune response in cells induced by a reference composition. In some embodiments, the cell is in a subject and the contacting comprises administering the cell to the subject. In some embodiments, the method comprises the step of administering to the subject a lipid nanoparticle comprising an ionizable lipid (e.g., a compound of Formula (I), (I-a), (I-b), (I-c), (I-d), (I-e), or (I-f)), a phospholipid, a structural lipid, a PEG lipid, and one or more therapeutic and/or prophylactic agents selected from a nucleotide, a polypeptide, and a nucleic acid (e.g., an RNA), wherein the lipid nanoparticle comprising an ionizable lipid (e.g., a compound of Formula (I), (I-a), (I-b), (I-c), (I-d), (I-e), or (I-f)) reduces the induction of the cellular immune response of the cells to the lipid nanoparticle comprising an ionizable lipid (e.g., a compound of Formula (I), (I-a), (I-b), (I-c), (I-d), (I-e), or (I-f)), as compared to the induction of the cellular immune response in cells induced by a reference composition. For example, the cellular immune response is an innate immune response, an adaptive immune response, or both.

In some embodiments, the disclosure provides a method of lowering immunogenicity in a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-III (e.g., PEG₂ₖ-DMG), and one or more therapeutic and/or prophylactic agents selected from a nucleotide, a polypeptide, and a nucleic acid (e.g., an RNA). For example, in some embodiments, the disclosure provides a method of lowering immunogenicity in a subject, wherein the method comprises the step of administering to the subject a lipid nanoparticle comprising a lipid of Table 1, DSPC, cholesterol, and PL-III (e.g., PEG₂ₖ-DMG), and one or more therapeutic and/or prophylactic agents selected from a nucleotide, a polypeptide, and a nucleic acid (e.g., an RNA).

In some embodiments, the disclosure provides a method of lowering immunogenicity in a subject, wherein the method comprises the step of administering to the subject a lipid nanoparticle comprising an ionizable lipid (e.g., a compound of Formula (I), (I-a), (I-b), (I-c), (I-d), (I-e), or (I-f)), DSPC, cholesterol, and PL-II (e.g., PEG-1), and one or more therapeutic and/or prophylactic agents selected from a nucleotide, a polypeptide, and a nucleic acid (e.g., an RNA). For example, in some embodiments, the disclosure provides a method of lowering immunogenicity in a subject, wherein the method comprises the step of administering to the subject a lipid nanoparticle comprising a lipid of Table 1, DSPC, cholesterol, and PL-II (e.g., PEG-1), and one or more therapeutic and/or prophylactic agents selected from a nucleotide, a polypeptide, and a nucleic acid (e.g., an RNA).

### Methods of Producing Polypeptides in Cells

The present disclosure provides methods of producing a polypeptide of interest in a cell (e.g., mammalian cell). Methods of producing polypeptides involve contacting a cell (e.g., mammalian cell) with an LNP including an mRNA encoding the polypeptide of interest. Upon contacting the cell with the nanoparticle composition, the mRNA may be taken up and translated in the cell to produce the polypeptide of interest.

In general, the step of contacting a cell (e.g., mammalian cell) with an LNP including an mRNA encoding a polypeptide of interest may be performed in vivo, ex vivo, in culture, or in vitro. The amount of LNP contacted with a cell, and/or the amount of mRNA therein, may depend on the type of cell or tissue being contacted, the means of administration, the physiochemical characteristics of the LNP and the mRNA (e.g., size, charge, and chemical composition) therein, and other factors. In general, an effective amount of the LNP will allow for efficient polypeptide production in the cell. Metrics for efficiency may include polypeptide translation (indicated by polypeptide expression), level of mRNA degradation, and immune response indicators.

The step of contacting an LNP including an mRNA with a cell may involve or cause transfection. A phospholipid including in the lipid component of an LNP may facilitate transfection and/or increase transfection efficiency, for example, by interacting and/or fusing with a cellular or intracellular membrane. Transfection may allow for the translation of the mRNA within the cell.

In some embodiments, the LNPs described herein may be used therapeutically. For example, an mRNA included in an LNP may encode a therapeutic polypeptide (e.g., in a translatable region) and produce the therapeutic polypeptide upon contacting and/or entry (e.g., transfection) into a cell. In other embodiments, an mRNA included in an LNP may encode a polypeptide that may improve or increase the immunity of a subject. For example, an mRNA may encode a granulocyte-colony stimulating factor or trastuzumab.

In certain embodiments, an mRNA included in an LNP may encode a recombinant polypeptide that may replace one or more polypeptides that may be substantially absent in a cell contacted with the nanoparticle composition. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

In some embodiments, contacting a cell with an LNP including an mRNA may reduce the innate immune response of a cell to an exogenous nucleic acid. A cell may be contacted with a first LNP including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

Methods of Delivering Therapeutic and/or Prophylactic Agents to Cells and Organs

The present disclosure provides methods of delivering a therapeutic and/or prophylactic agent to a cell (e.g., mammalian cell) or organ. Delivery of a therapeutic and/or prophylactic agent to a cell involves administering an LNP including the therapeutic and/or prophylactic agent to a subject, where administration of the composition involves contacting the cell with the composition. For example, a protein, cytotoxic agent, radioactive ion, chemotherapeutic and/or prophylactic agent, or nucleic acid (such as an RNA, e.g., mRNA) may be delivered to a cell or organ. In the instance that a therapeutic and/or prophylactic agent is an mRNA, upon contacting a cell with the nanoparticle composition, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, an LNP may target a particular type or class of cells (e.g., cells of a particular organ or system thereof). For example, an LNP including a therapeutic and/or prophylactic agent of interest may be specifically delivered to a mammalian liver, kidney, spleen, or lung. Specific delivery to a particular class of cells, an organ, or a system or group thereof implies that a higher proportion of lipid nanoparticles (e.g., loaded LNPs) including a therapeutic and/or prophylactic agent are delivered to the destination (e.g., tissue) of interest relative to other destinations. In some embodiments, specific delivery of a loaded LNP comprising an mRNA may result in a greater than 2 fold, 5 fold, 10 fold, 15 fold, or 20 fold increase in mRNA expression in cells of the targeted destination (e.g., tissue of interest, such as a liver) as compared to cells of another destination (e.g., the spleen). In some embodiments, the tissue of interest is selected from the group consisting of a liver, a kidney, a lung, a spleen, and tumor tissue (e.g., via intratumoral injection).

In some embodiments, delivery of an mRNA comprised in a loaded LNP of the disclosure (i.e., a lipid nanoparticle formulated with a lipid of the disclosure) results in a greater than 2 fold, 5 fold, 10 fold, 15 fold, or 20 fold increase in mRNA expression as compared to delivery of an mRNA comprised in an LNP formulated with another lipid (i.e., without any of the ionizable lipids described herein (e.g., compounds of Formulae (I), (I-a), (I-b), (I-c), (I-d), (I-e), and (I-f)).

As another example of targeted or specific delivery, an mRNA that encodes a protein-binding partner (e.g., an antibody or functional fragment thereof, a scaffold protein, or a peptide) or a receptor on a cell surface may be included in a nanoparticle composition. An mRNA may additionally or instead be used to direct the synthesis and extracellular localization of lipids, carbohydrates, or other biological moieties. Alternatively, other therapeutic and/or prophylactic agents or elements (e.g., lipids or ligands) of an LNP may be selected based on their affinity for particular receptors (e.g., low density lipoprotein receptors) such that an LNP may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)2 fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tribodies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In some embodiments, multiple antibodies are expressed on the surface of a cell, and each antibody can have a different specificity for a desired target. Such approaches can increase the avidity and specificity of targeting interactions. A ligand can be selected, e.g., by a person skilled in the biological arts, based on the desired localization or function of the cell.

Targeted cells may include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells.

In some embodiments, an LNP may target hepatocytes. Apolipoprotiens such as apolipoprotein E (apoE) have been shown to associate with neutral or near neutral lipid-containing LNPs in the body, and are known to associate with receptors such as low-density lipoprotein receptors (LDLRs) found on the surface of hepatocytes. Thus, an LNP including a lipid component with a neutral or near neutral charge that is administered to a subject may acquire apoE in a subject's body and may subsequently deliver a therapeutic and/or prophylactic agent (e.g., an RNA) to hepatocytes including LDLRs in a targeted manner.

### Methods of Treating or Preventing Diseases and Disorders

LNPs may be useful for treating or preventing a disease, disorder, or condition. In particular, such compositions may be useful in treating or preventing a disease, disorder, or condition characterized by missing or aberrant protein or polypeptide activity. LNPs may be useful for treating a disease, disorder, or condition. In particular, such compositions may be useful in treating a disease, disorder, or condition characterized by missing or aberrant protein or polypeptide activity. LNPs may be useful for preventing a disease, disorder, or condition. In particular, such compositions may be useful in preventing a disease, disorder, or condition characterized by missing or aberrant protein or polypeptide activity. For example, an LNP comprising an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may be useful in the treatment and prevention of acute diseases, disorders, or conditions such as sepsis, stroke, and myocardial infarction. Because translation may occur rapidly, the methods and compositions may be useful in the treatment of acute diseases, disorders, or conditions such as sepsis, stroke, and myocardial infarction. Because translation may occur rapidly, the methods and compositions may be useful in the prevention of acute diseases, disorders, or conditions such as sepsis, stroke, and myocardial infarction. A therapeutic and/or prophylactic agent included in an LNP may also be capable of altering the rate of transcription of a given species, thereby affecting gene expression.

Diseases, disorders, and/or conditions characterized by dysfunctional or aberrant protein or polypeptide activity for which a composition may be administered include, but are not limited to, rare diseases, infectious diseases (as both vaccines and therapeutics), cancer and proliferative diseases, genetic diseases, autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. The present disclosure provides a method for treating and preventing such diseases, disorders, and/or conditions in a subject by administering an LNP including an RNA and a lipid component including an ionizable lipid (e.g., a compound of Formula (I), (I-a), (I-b), (I-c), (I-d), (I-e), or (I-f)), a phospholipid (optionally unsaturated), a PEG lipid, and a structural lipid, wherein the RNA may be an mRNA encoding a polypeptide that antagonizes or otherwise overcomes an aberrant protein activity present in the cell of the subject. The present disclosure provides a method for treating such diseases, disorders, and/or conditions in a subject by administering an LNP including an RNA and a lipid component including an ionizable lipid (e.g., a compound of Formula (I), (I-a), (I-b), (I-c), (I-d), (I-e), or (I-f)), a phospholipid (optionally unsaturated), a PEG lipid, and a structural lipid, wherein the RNA may be an mRNA encoding a polypeptide that antagonizes or otherwise overcomes an aberrant protein activity present in the cell of the subject. The present disclosure provides a method for preventing such diseases, disorders, and/or conditions in a subject by administering an LNP including an RNA and a lipid component including an ionizable lipid (e.g., a compound of Formula (I), (I-a), (I-b), (I-c), (I-d), (I-e), or (I-f)), a phospholipid (optionally unsaturated), a PEG lipid, and a structural lipid, wherein the RNA may be an mRNA encoding a polypeptide that antagonizes or otherwise overcomes an aberrant protein activity present in the cell of the subject.

The disclosure provides methods involving administering LNPs including one or more therapeutic and/or prophylactic agents and pharmaceutical compositions including the same. The terms therapeutic and prophylactic can be used interchangeably herein with respect to features and embodiments of the present disclosure. Therapeutic compositions, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identify of a disorder being treated, if any; the one or more therapeutic and/or prophylactic agents employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A loaded LNP may be administered by any route. In some embodiments, compositions, including prophylactic, diagnostic, or imaging compositions including one or more loaded LNPs described herein, are administered by one or more of a variety of routes, including oral, intravenous, intramuscular, intra-arterial, subcutaneous, trans- or intra-dermal, interdermal, intraperitoneal, mucosal, nasal, intratumoral, intranasal; by inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by any other parenteral route of administration or by inhalation. However, the present disclosure encompasses the delivery or administration of compositions described herein by any appropriate route taking into consideration likely advances in the sciences of drug delivery. In general, the most appropriate route of administration will depend upon a variety of factors including the nature of the loaded LNP including one or more therapeutic and/or prophylactic agents (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a therapeutic and/or prophylactic agent (e.g., an mRNA) in a given dose, where a dose of 1 mg/kg (mpk) provides 1 mg of a therapeutic and/or prophylactic agent per 1 kg of subject body weight. In some embodiments, a dose of about 0.001 mg/kg to about 10 mg/kg of a therapeutic and/or prophylactic agent of a loaded LNP may be administered. In other embodiments, a dose of about 0.005 mg/kg to about 2.5 mg/kg of a therapeutic and/or prophylactic agent may be administered. In certain embodiments, a dose of about 0.1 mg/kg to about 1 mg/kg may be administered. In other embodiments, a dose of about 0.05 mg/kg to about 0.25 mg/kg may be administered. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

LNPs including one or more therapeutic and/or prophylactic agents may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more LNPs including one or more different therapeutic and/or prophylactic agents may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic and/or prophylactic agent), or they may achieve different effects (e.g., control of any adverse effects, such as infusion related reactions).

AN LNP may be used in combination with an agent to increase the effectiveness and/or therapeutic window of the composition. Such an agent may be, for example, an anti-inflammatory compound, a steroid (e.g., a corticosteroid), a statin, an estradiol, a BTK inhibitor, an S1P1 agonist, a glucocorticoid receptor modulator (GRM), or an anti-histamine. In some embodiments, an LNP may be used in combination with dexamethasone, methotrexate, acetaminophen, an H1 receptor blocker, or an H2 receptor blocker. In some embodiments, a method of treating a subject in need thereof or of delivering a therapeutic and/or prophylactic agent to a subject (e.g., a mammal) may involve pre-treating the subject with one or more agents prior to administering a nanoparticle composition. For example, a subject may be pre-treated with a useful amount (e.g., 10 mg, 20 mg, 30 mg, 40 mg, 50 mg, 60 mg, 70 mg, 80 mg, 90 mg, 100 mg, or any other useful amount) of dexamethasone, methotrexate, acetaminophen, an H1 receptor blocker, or an H2 receptor blocker. Pre-treatment may occur 24 or fewer hours (e.g., 24 hours, 20 hours, 16 hours, 12 hours, 8 hours, 4 hours, 2 hours, 1 hour, 50 minutes, 40 minutes, 30 minutes, 20 minutes, or 10 minutes) before administration of the LNP and may occur one, two, or more times in, for example, increasing dosage amounts.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

The examples provided below include procedures, intermediates, and characterization data useful, e.g., for the preparation of compounds provided herein. All synthetic steps, procedures, compounds (e.g., synthetic intermediates), reaction conditions, reaction mixtures, reagents, etc. are included herein as aspects of the present disclosure.

### Example 1: Synthesis of Malonate-Containing Lipids

Compounds provided herein can be prepared, for example, and shown in Scheme 1 and Schemes 2 and 3 below.

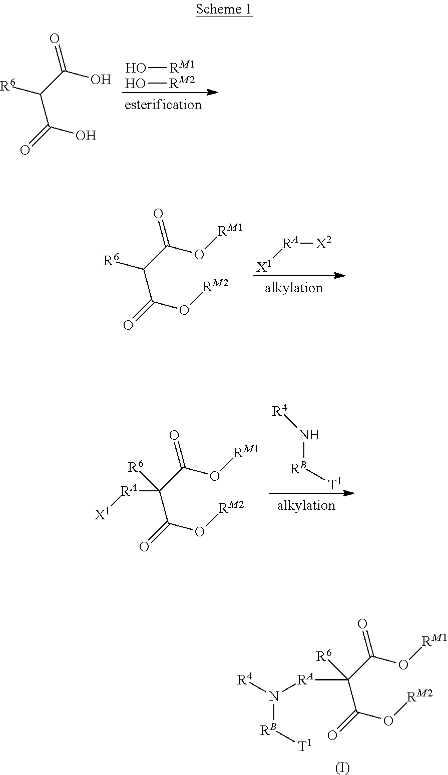

X¹, X²=each independently halogen or a leaving group
   Dioctyl malonate

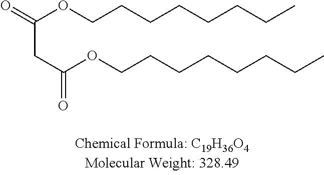 [SMILES: CCCCCCCCOC(=O)CC(=O)OCCCCCCCC]

To a round bottom flask equipped with a stir bar was added malonic acid (2.00 g, 19.3 mmol) and DCM (192 mL). To the resulting mixture was added 1-octanol (6.63 mL, 42.3 mmol), DMAP (1.17 g, 9.61 mmol), and EDCl (9.21 g, 48.0 mmol). The resulting mixture was allowed to stir at room temperature overnight. The reaction was then diluted with water and the layers were separated. The aqueous layer was extracted with DCM. The combined organics were dried (MgSO₄), filtered, and concentrated. The crude residue was purified by silica gel chromatography (0-10-20% EtOAc:hexanes) to give dioctyl malonate (4.90 g, 78%) as a clear oil. ¹H NMR (300 MHz, CDCl₃) δ: ppm 4.21-4.00 (m, 4H); 3.42-3.32 (m, 1H); 2.10-2.00 (m, 1H); 1.74-1.53 (m, 4H); 1.45-1.18 (m, 20H); 0.98-0.80 (m, 6H).

Dioctyl 2-(6-bromohexyl)malonate

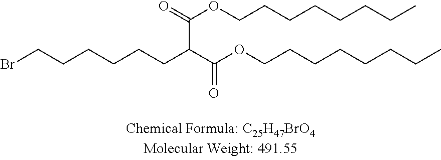 [SMILES: CCCCCCCCOC(=O)C(CCCCCCBr)C(=O)OCCCCCCCC]

A mixture of 1,6-dibromohexane (1.23 mL, 8.00 mmol), benzyltriethylammonium chloride (5.00 mg, 24.0 μmol), and potassium carbonate (1.66 g, 12.0 mmol) was heated at 100° C. 1,3-dioctyl propanedioate (5.09 mL, 12.0 mmol) was then added dropwise and the resulting mixture was allowed to stir for 2 h at 100° C. The reaction mixture was then cooled to rt, filtered with DCM, and concentrated by rotary evaporator with the heating bath at 95° C. The crude residue was purified by silica gel chromatography (0-20% EtOAc:hexanes) to give dioctyl 2-(6-bromohexyl)malonate (549 mg, 14%) as a clear viscous oil. ¹H NMR (300 MHz, CDCl₃) δ: ppm 4.12 (ddd, 4H, J=6.0, 6.0, 3.0 Hz); 3.39 (t, 2H, J=6.0 Hz); 3.31 (t, 1H, J=9.0 Hz); 1.95-1.78 (m, 4H); 1.69-1.56 (m, 4H); 1.50-1.19 (m, 26H); 0.93-0.83 (m, 6H).

Dihexyl 2-(6-bromohexyl)malonate

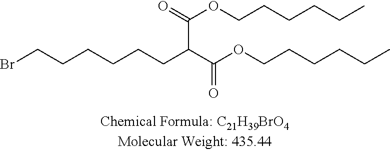 [SMILES: CCCCCCOC(=O)C(CCCCCCBr)C(=O)OCCCCCC]

A mixture of 1,6-dibromohexane (0.941 mL, 6.12 mmol), benzyltriethylammonium chloride (8.00 mg, 37.0 μmol), and potassium carbonate (2.54 g, 18.4 mmol) was heated at 100° C. 1,3-dihexyl propanedioate (5.20 mL, 18.4 mmol) was then added dropwise and the resulting mixture was allowed to stir for 2 h at 100° C. The reaction mixture was then cooled to rt, filtered, and concentrated by rotary evaporator with the heating bath at 95° C. The crude residue was purified by silica gel chromatography (0-20% EtOAc:hexanes) to give dihexyl 2-(6-bromohexyl)malonate (488 mg, 18%) as a clear viscous oil. ¹H NMR (300 MHz, CDCl₃) δ: ppm 4.12 (ddd, 4H, J=9.0, 9.0, 3.0 Hz); 3.39 (t, 2H, J=6.0 Hz); 3.32 (t, 1H, J=6.0 Hz); 1.94-1.79 (m, 4H); 1.69-1.56 (m, 4H); 1.51-1.20 (m, 18H); 0.93-0.83 (m, 6H).

### Compound M1: Dihexyl 2-(6-((8-(heptadecan-9-yloxy)-8-oxooctyl)(2-hydroxyethyl)amino)hexyl)malonate

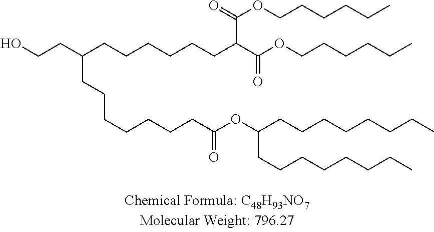 [SMILES: CCCCCCCCC(CCCCCCCC)OC(=O)CCCCCCCC(CCO)CCCCCCC(C(=O)OCCCCCC)C(=O)OCCCCCC]

To a solution of dihexyl 2-(6-bromohexyl)malonate (207 mg, 0.48 mmol) and heptadecan-9-yl 8-[(2-hydroxyethyl)amino]octanoate (200 mg, 0.45 mmol) in cyclopentyl methyl ether (2.00 mL) and acetonitrile (2.00 mL) was added potassium iodide (83.0 mg, 0.50 mmol) and potassium carbonate (375 mg, 2.72 mmol). The reaction was allowed to stir at 80° C. for 16 h. After reaction completion as indicated by LC/MS and TLC analysis, the solvent was removed under vacuum. The resulting residue was diluted with DCM and washed with water. The organic layer was separated, washed with brine, dried (MgSO₄), filtered, and concentrated. The crude residue was purified by silica gel chromatography (0-100% 80:20:1 DCM:MeOH:NH₄OH/DCM) to afford dihexyl 2-(6-((8-(heptadecan-9-yloxy)-8-oxooctyl)(2-hydroxyethyl)amino)hexyl)malonate (226 mg, 63%) as a light-yellow transparent oil. UPLC/ELSD: RT=2.71 min. MS(ES): m/z (MH⁺) 796.87 for C₄₈H₉₃NO₇. ¹H NMR (300 MHz, CDCl₃) δ: ppm 4.83 (pentet, 1H, J=6.0 Hz); 4.08 (dt, 4H, J=6.0, 6.0, 3.0 Hz); 3.49 (t, 2H, J=6.0 Hz); 3.28 (t, 1H, J=6.0 Hz); 3.34-3.12 (br s, 1H); 2.54 (t, 2H, J=6.0 Hz); 2.41 (t, 4H, J=6.0 Hz); 2.24 (t, 2H, J=9.0 Hz); 1.85 (br dt, 2H, J=6.0, 6.0 Hz); 1.66-1.52 (m, 6H); 1.52-1.34 (m, 9H); 1.34-1.13 (m, 46H), 0.92-0.76 (m, 12H).

### Compound M2: Dioctyl 2-(6-((8-(heptadecan-9-yloxy)-8-oxooctyl)(2-hydroxyethyl)amino)hexyl)malonate

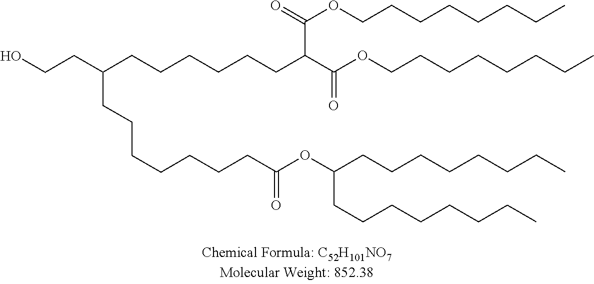 [SMILES: CCCCCCCCOC(=O)C(CCCCCCC(CCO)CCCCCCCC(=O)OC(CCCCCCCC)CCCCCCCC)C(=O)OCCCCCCCC]

To a solution of dioctyl 2-(6-bromohexyl)malonate (160 mg, 0.33 mmol) and heptadecan-9-yl 8-[(2-hydroxyethyl)amino]octanoate (137 mg, 0.31 mmol) in cyclopentyl methyl ether (1.40 mL) and acetonitrile (1.40 mL) was added potassium iodide (57.0 mg, 0.34 mmol) and potassium carbonate (257 mg, 1.86 mmol). The reaction was allowed to stir at 80° C. for 16 h. After reaction completion as indicated by LC/MS and TLC analysis, the solvent was removed under vacuum. The resulting residue was diluted with DCM and washed with water. The organic layer was separated, washed with brine, dried (MgSO₄), filtered, and concentrated. The crude residue was purified by silica gel chromatography (0-100% 80:20:1 DCM:MeOH:NH₄OH/DCM) to afford dioctyl 2-(6-((8-(heptadecan-9-yloxy)-8-oxooctyl) (2-hydroxyethyl)amino)hexyl)malonate (205 mg, 78%) as a clear viscous oil. UPLC/ELSD: RT=2.85 min. MS(ES): m/z (MH⁺) 852.24 for C₅₂H₁₀₁NO₇. ¹H NMR (300 MHz, CDCl₃) δ: ppm 4.86 (pentet, 1H, J=6.0 Hz); 4.12 (dt, 4H, J=6.0, 3.0 Hz); 3.53 (br t, 2H, J=6.0 Hz); 3.31 (t, 1H, J=9.0 Hz); 3.01 (br s, 1H); 2.58 (br t, 2H, J=6.0 Hz); 2.44 (br t, 4H, J=6.0 Hz); 2.28 (t, 2H, J=6.0 Hz); 1.88 (br q, 2H, J=6.0 Hz); 1.70-1.55 (m, 6H); 1.55-1.18 (m, 63H); 0.98-0.79 (m, 12H).

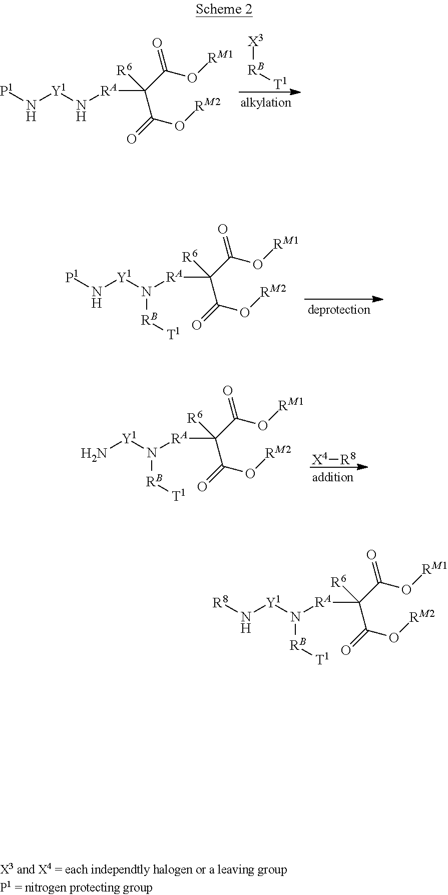

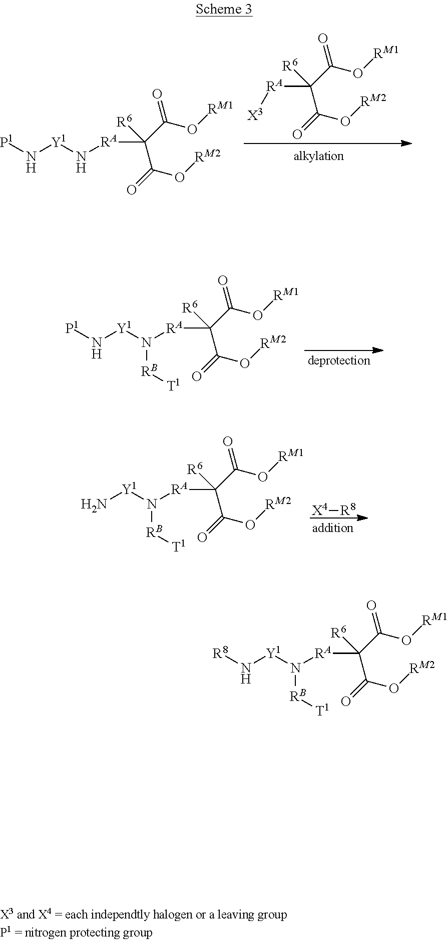

Dihexyl 2-(6-((3-((tert-butoxycarbonyl)amino)propyl)amino)hexyl)malonate

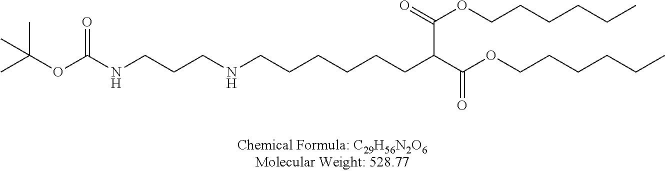 [SMILES: CCCCCCOC(=O)C(CCCCCCNCCCNC(=O)OC(C)(C)C)C(=O)OCCCCCC]

To a solution of N-Boc-1,3-propanediamine (381 mg, 2.19 mmol) in EtOH (2.3 mL) heated to 65° C. was added a solution of dihexyl 2-(6-bromohexyl)malonate (272 mg, 0.63 mmol) in EtOH (1.2 mL) over 0.25h. The reaction was stirred at 65° C. for 16 h. The reaction was then cooled room temperature and EtOH was removed via vacuum. The crude material was dissolved in EtOAc and washed with saturated aqueous NaHCO₃. The EtOAc layer was washed with brine, dried over MgSO₄, filtered, and concentrated. The crude residue was purified by silica gel chromatography (0-100% 80:20:1 DCM:MeOH:NH₄OH/DCM) to afford dihexyl 2-(6-((3-((tert-butoxycarbonyl)amino)propyl)amino)hexyl) malonate (247 mg, 75%) as a yellow transparent oil. ¹H NMR (300 MHz, CDCl₃) δ: ppm 5.15 (br s, 1H); 4.11 (dt, 4H, J=6.0, 3.0 Hz); 3.31 (t, 1H, J=6.0 Hz); 3.19 (br q, 2H, J=2H); 2.65 (t, 2H, J=9.0 Hz); 2.56 (t, 2H, J=6.0 Hz); 1.88 (br q, 2H, J=6.0 Hz); 1.70-1.56 (m, 6H); 1.50-1.40 (br m, 2H); 1.44 (s, 9H); 1.39-1.20 (m, 18H); 0.93-0.83 (m, 6H).

Dihexyl 2-(6-((3-((tert-butoxycarbonyl)amino)propyl)(8-(heptadecan-9-yloxy)-8-oxooctyl)amino)hexyl)malonate

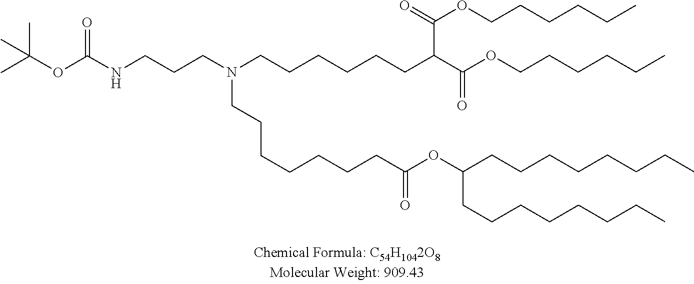 [SMILES: CCCCCCCCC(CCCCCCCC)OC(=O)CCCCCCCN(CCCCCCC(C(=O)OCCCCCC)C(=O)OCCCCCC)CCCNC(=O)OC(C)(C)C]

To a solution of dihexyl 2-(6-((3-((tert-butoxycarbonyl)amino)propyl)amino)hexyl)malonate (247 mg, 0.47 mmol) and heptadecan-9-yl 8-bromooctanoate (226 mg, 0.49 mmol) in propionitrile (1.30 mL) was added potassium carbonate (97 mg, 0.70 mmol) and potassium iodide (12 mg, 0.070 mmol). The reaction was heated to 80° C. and allowed to stir overnight. After reaction completion as indicated by LC/MS and TLC analysis, the reaction cooled to room temperature and vacuum filtered. The reaction residue and filtered solid washed with propionitrile, and the filtrate was concentrated in vacuo at 40° C. The crude product was dissolved in heptane and washed twice with acetonitrile. Upon separation of layers, the heptane layer was collected, dried with MgSO₄, filtered, and concentrated. The crude residue was purified by silica gel chromatography (0-100% 80:20:1 DCM:MeOH:NH₄OH/DCM) to afford dihexyl 2-(6-((3-((tert-butoxycarbonyl)amino)propyl)(8-(heptadecan-9-yloxy)-8-oxooctyl)amino)hexyl)malonate (190 mg, 45%) as a yellow transparent oil. ¹H NMR (300 MHz, CDCl₃) δ: ppm 5.63 (br s, 1H); 4.86 (pentet, 1H, J=6.0 Hz); 4.12 (dt, 4H, J=6.0, 3.0 Hz); 3.31 (t, 1H, J=9.0 Hz); 3.17 (br q, 2H, J=6.0 Hz); 2.43 (br t, 2H, J=9.0 Hz); 2.34 (br t, 4H, J=6.0 Hz); 2.27 (t, 2H, J=6.0 Hz); 1.88 (br q, 2H, J=6.0 Hz); 1.69-1.55 (m, 8H); 1.55-1.46 (m, 5H); 1.45-1.38 (s, 11H); 1.37-1.17 (m, 48H); 0.95-0.82 (m, 12H).

Dihexyl 2-(6-((3-aminopropyl)(8-(heptadecan-9-yloxy)-8-oxooctyl)amino)hexyl)malonate

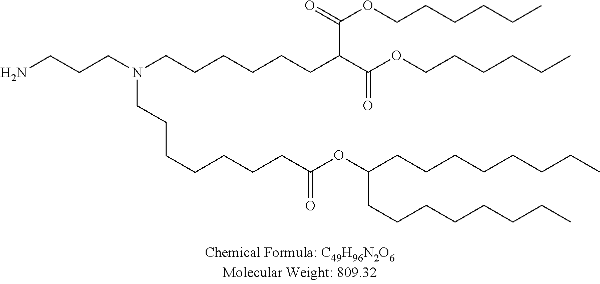 [SMILES: CCCCCCCCC(CCCCCCCC)OC(=O)CCCCCCCN(CCCN)CCCCCCC(C(=O)OCCCCCC)C(=O)OCCCCCC]

To a solution of dihexyl 2-(6-((3-((tert-butoxycarbonyl)amino)propyl)(8-(heptadecan-9-yloxy)-8-oxooctyl)amino)hexyl)malonate (190 mg, 0.209 mmol) in methylene chloride (4.2 mL) was added trifluoroacetic acid (320 μL, 4.18 mmol) dropwise via addition funnel. The reaction mixture was allowed to stir at room temperature for 4 h prior to monitoring by TLC and LCMS for completion. The reaction was then concentrated in vacuo and carefully quenched with saturated aqueous NaHCO₃ and diluted further with DCM. The layers were separated, and the aqueous layer was extracted with DCM. The combined organics were then washed with brine, dried (MgSO₄), filtered, and concentrated. The crude residue was purified by silica gel chromatography (0-100% 80:20:1 DCM:MeOH:NH₄OH/DCM) to afford dihexyl 2-(6-((3-aminopropyl)(8-(heptadecan-9-yloxy)-8-oxooctyl)amino)hexyl)malonate (124 mg, 73%) as a yellow transparent oil. ¹H NMR (300 MHz, CDCl₃) δ: ppm 5.21 (br s, 2H); 4.84 (pentet, 1H, J=6.0 Hz); 4.09 (dt, 4H, J=6.0, 3.0 Hz); 3.29 (t, 1H, J=6.0 Hz); 2.89 (br t, 2H, J=6.0 Hz); 2.53 (t, 2H, J=6.0 Hz); 2.39 (br t, 4H, J=6.0 Hz); 2.25 (t, 2H, J=6.0 Hz); 1.85 (br q, 2H, J=9.0 Hz); 1.73-1.53 (m, 8H); 1.52-1.12 (m, 56H); 0.95-0.75 (m, 12H).

### Compound M3: Dihexyl 2-(6-((8-(heptadecan-9-yloxy)-8-oxooctyl)(3-((2-(methylamino)-3,4-dioxocyclobut-1-en-1-yl)amino)propyl)amino)hexyl)malonate

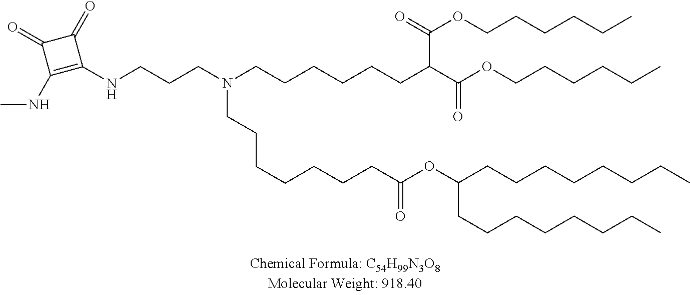 [SMILES: CCCCCCCCC(CCCCCCCC)OC(=O)CCCCCCCN(CCCCCCC(C(=O)OCCCCCC)C(=O)OCCCCCC)CCCNc1c(NC)c(=O)c1=O]

A round bottom flask equipped with a stir bar was charged with dihexyl 2-(6-((3-aminopropyl)(8-(heptadecan-9-yloxy)-8-oxooctyl)amino)hexyl)malonate (124 mg, 0.15 mmol), 3-methoxy-4-(methylamino)cyclobut-3-ene-1,2-dione (28 mg, 0.20 mmol), and 2-Methyl THF (1 mL). To this reaction mixture was added 10% aqueous potassium carbonate (1 mL). The resulting mixture was heated to 45° C. and stirred overnight. After confirmation of conversion by LC/MS and TLC (10% MeOH:DCM 1% NH₄OH), The reaction cooled to room temperature and diluted with water. Organic layer was separated, and the aqueous layer was extracted 2× with heptane. The organic layers were combined, washed 3× with a 1:1 acetonitrile/water mixture, dried (MgSO₄), filtered, and concentrated in vacuo. The crude residue was azeotroped with MeOH and concentrated, and this process was repeated 3× in total. The crude residue was purified by silica gel chromatography (0-100% 80:20:1 DCM:MeOH:NH₄OH/DCM) to afford dihexyl 2-(6-((8-(heptadecan-9-yloxy)-8-oxooctyl)(3-((2-(methylamino)-3,4-dioxocyclobut-1-en-1-yl)amino)propyl)amino)hexyl)malonate (42 mg, 30%) as a white waxy solid. UPLC/ELSD: RT=2.70 min. MS(ES): m/z (MH⁺) 919.46 for C₅₄H₉₉N₃O₈. ¹H NMR (300 MHz, CDCl₃) δ: ppm 7.32 (br s, 1H); 4.84 (pentet, 1H, J=6.0 Hz); 4.11 (dt, 4H, J=6.0, 3.0 Hz); 3.64 (br s, 2H); 3.31 (t, 1H, J=9.0 Hz); 3.27 (d, 3H, J=6.0 Hz); 2.54 (br t, 2H, J=6.0 Hz); 2.41 (br t, 4H, J=6.0 Hz); 2.27 (t, 2H, J=9.0 Hz); 1.87 (br q, 2H, J=9.0 Hz); 1.75 (br t, 2H, J=6.0 Hz); 1.69-1.55 (m, 6H); 1.55-1.15 (m, 57H); 0.94-0.80 (m, 12H).

### Compound M4: Dihexyl 2-(6-((3-acetamidopropyl)(8-(heptadecan-9-yloxy)-8-oxooctyl)amino)hexyl)malonate

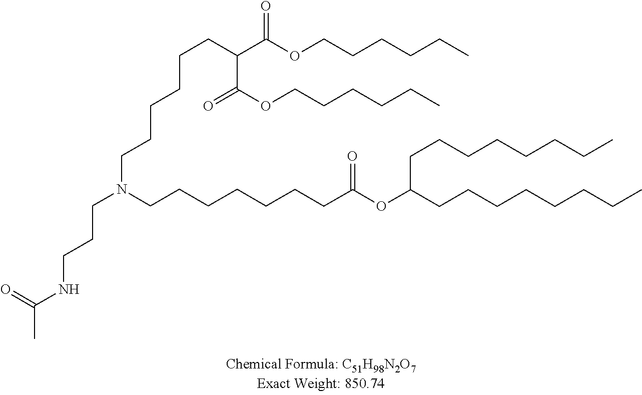 [SMILES: CCCCCCCCC(CCCCCCCC)OC(=O)CCCCCCCN(CCCCCCC(C(=O)OCCCCCC)C(=O)OCCCCCC)CCCNC(C)=O]

To a solution of 1,3-dihexyl 2-{6-[(3-aminopropyl) [8-(heptadecan-9-yloxy)-8-oxooctyl]amino]hexyl}propanedioate (0.25 g, 0.31 mmol) in DCM (2 mL) added acetyl chloride (0.033 mL, 0.463 mmol) and triethylamine (0.109 mL, 0.772 mmol). The reaction was allowed to stir at rt for 2 h. The residue was diluted with DCM and extracted with water. The organic layer was separated, washed with brine, dried with Na₂SO₄, filtered, and evaporated under vacuum. The residue was purified by flash chromatography (ISCO) by 0-100% (a solution of 20% MeOH, 80% DCM, 1% NH₄OH) in DCM to obtain dihexyl 2-(6-((3-acetamidopropyl)(8-(heptadecan-9-yloxy)-8-oxooctyl)amino)hexyl)malonate in 64% yield (0.174 g, 0.198 mmol). UPLC/ELSD: RT=2.81 min. MS (ES): m/z (MH⁺) 851.67 for C₅₁H₉₈N₂O₇. ¹H NMR (300 MHz, CDCl₃) δ: ppm 7.21 (bs, 1H); 4.88 (m, 1H); 4.15 (m, 4H); 3.45-3.27 (m, 3H); 3.19-2.82 (bm, 6H); 2.30 (t, 2H); 2.17-1.18 (m, 69H); 0.91 (m, 12H).

### Compound M5: Dihexyl 2-(6-((8-(heptadecan-9-yloxy)-8-oxooctyl)(3-propionamidopropyl)amino)hexyl)malonate

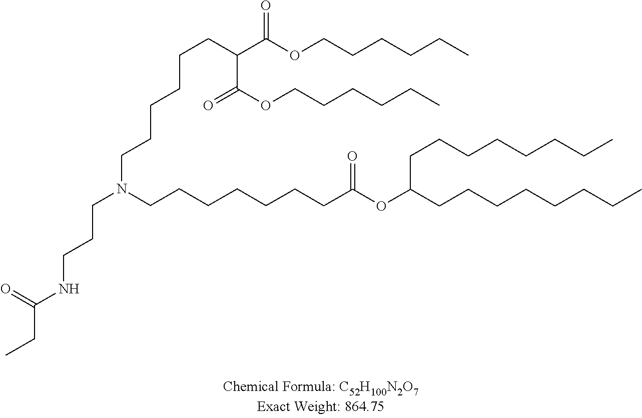 [SMILES: CCCCCCCCC(CCCCCCCC)OC(=O)CCCCCCCN(CCCCCCC(C(=O)OCCCCCC)C(=O)OCCCCCC)CCCNC(=O)CC]

Prepared as described for compound M4 using propionyl chloride as the acylating reagent. UPLC/ELSD: RT=2.84 min. MS(ES): m/z (MH⁺) 865.82 for C₅₂H₁₀₀N₂O₇. ¹H NMR (300 MHz, CDCl₃) δ: ppm 4.89 (m, 1H); 4.13 (m, 4H); 3.34 (m, 3H); 2.60-2.23 (m, 8H); 2.18 (m; 2H); 1.91 (m, 2H); 1.74-1.05 (m, 67H); 0.92 (m, 12H).

### Compound M6: Dihexyl 2-(6-((3-(cyclopropanecarboxamido)propyl)(8-(heptadecan-9-yloxy)-8-oxooctyl)amino)hexyl)malonate

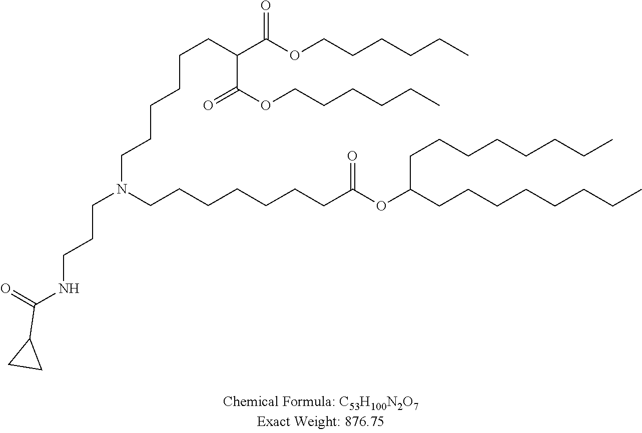 [SMILES: CCCCCCCCC(CCCCCCCC)OC(=O)CCCCCCCN(CCCCCCC(C(=O)OCCCCCC)C(=O)OCCCCCC)CCCNC(=O)C1CC1]

Prepared as described for compound M4 using cyclopropanecarbonyl chloride as the acylating reagent. UPLC/ELSD: RT=2.84 min. MS(ES): m/z (MH⁺) 877.58 for C₅₃H₁₀₀N₂O₇. ¹H NMR (300 MHz, CDCl₃) δ: ppm 7.48 (bs, 1H); 4.88 (m, 1H); 4.13 (m, 4H); 3.47-3.26 (m, 3H); 3.16-2.84 (bm, 3H); 2.61-2.24 (m, 5H); 2.09 (bm, 1H); 1.98-1.17 (m, 66H); 0.92 (m, 14H); 0.73 (bm, 2H).

### Compound M12: 1,3-dihexyl 2-{6-[(3-{[2-(methylamino)-3,4-dioxocyclobut-1-en-1-yl]amino}propyl)[8-oxo-8-(tridecan-7-yloxy)octyl]amino]hexyl}propanedioate

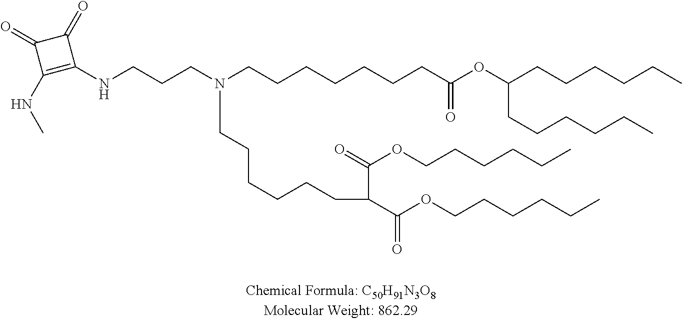 [SMILES: CCCCCCOC(=O)C(CCCCCCN(CCCCCCCC(=O)OC(CCCCCC)CCCCCC)CCCNc1c(NC)c(=O)c1=O)C(=O)OCCCCCC]

For procedure see Compound M3: Dihexyl 2-(6-((8-(heptadecan-9-yloxy)-8-oxooctyl)(3-((2-(methylamino)-3,4-dioxocyclobut-1-en-1-yl)amino)propyl)amino)hexyl)malonate. UPLC/ELSD: RT=13.113. MS(ES): m/z (MH⁺) 863.96. ¹H NMR (300 MHz, CD₃OD) δ 4.08 (td, J=6.4, 5.3 Hz, 4H), 3.56 (d, J=9.7 Hz, 2H), 3.21 (s, 2H), 2.56-2.43 (m, 3H), 2.40 (d, J=7.8 Hz, 2H), 2.27 (t, J=7.2 Hz, 2H), 1.77 (dq, J=21.6, 7.2 Hz, 4H), 1.58 (q, J=6.9 Hz, 6H), 1.47 (dd, J=15.0, 7.3 Hz, 7H), 1.37-1.23 (m, 35H), 0.87 (td, J=5.8, 3.9 Hz, 12H).

### 1,3-dihexyl 2-{6-[(3-aminopropyl)[8-oxo-8-(tridecan-7-yloxy) octyl]amino]hexyl}propanedioate

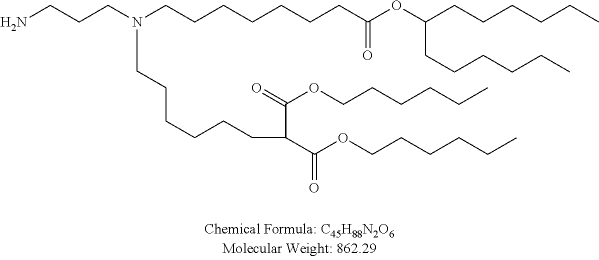 [SMILES: CCCCCCOC(=O)C(CCCCCCN(CCCN)CCCCCCCC(=O)OC(CCCCCC)CCCCCC)C(=O)OCCCCCC]

For procedure see Dihexyl 2-(6-((3-aminopropyl)(8-(heptadecan-9-yloxy)-8-oxooctyl)amino)hexyl)malonate. MS(ES): m/z (M+) 753.58. ¹H NMR (300 MHz, CD₃OD) δ 4.12 (q, J=6.3 Hz, 4H), 2.85 (t, J=7.0 Hz, 2H), 2.53 (dt, J=30.0, 7.5 Hz, 6H), 2.31 (t, J=7.2 Hz, 2H), 1.86 (d, J=8.2 Hz, 2H), 1.67 (dt, J=23.0, 6.5 Hz, 17H), 1.41-1.26 (m, 40H), 1.25-1.11 (m, 4H), 0.91 (td, J=6.3, 4.0 Hz, 12H).

### 1,3-dihexyl 2-[6-({3-[(tert-butoxycarbonyl)amino]propyl}[8-oxo-8-(tridecan-7-yloxy)octyl]amino)hexyl]propanedioate

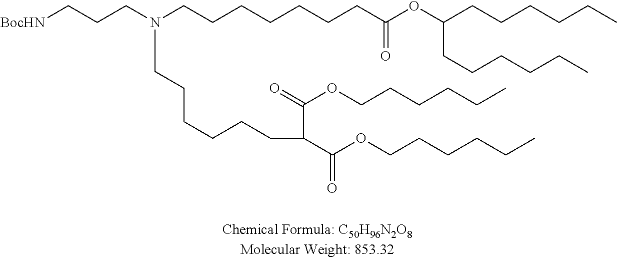 [SMILES: CCCCCCOC(=O)C(CCCCCCN(CCCCCCCC(=O)OC(CCCCCC)CCCCCC)CCCNC(=O)OC(C)(C)C)C(=O)OCCCCCC]

For procedure see Dihexyl 2-(6-((3-((tert-butoxycarbonyl)amino)propyl)(8-(heptadecan-9-yloxy)-8-oxooctyl)amino)hexyl)malonate. UPLC/ELSD: RT=8.301 min. MS(ES): m/z (MH⁺) 854.09. ¹H NMR (300 MHz, CDCl₃) δ 4.11 (td, J=6.7, 2.5 Hz, 4H), 3.66 (s, 2H), 3.37-3.23 (m, 4H), 2.63 (s, 2H), 2.49 (t, J=7.7 Hz, 4H), 2.28 (t, J=7.4 Hz, 2H), 1.94-1.74 (m, 4H), 1.63 (p, J=6.7 Hz, 6H), 1.54-1.39 (m, 8H), 1.38-1.17 (m, 44H), 0.96-0.82 (m, 12H).

### 1,3-dihexyl 2-[6-({3-[(tert-butoxycarbonyl)amino]propyl}amino)hexyl]propanedioate

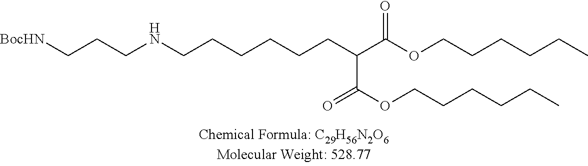 [SMILES: CCCCCCOC(=O)C(CCCCCCNCCCNC(=O)OC(C)(C)C)C(=O)OCCCCCC]

To a 100 mL round bottom flask equipped with a 60 mL addition funnel, and tert-butyl N-(3-aminopropyl) carbamate (5.602 g, 32.151 mmol, 3.5 equiv.) was added EtOH (30.62 mL, 0.3 M) and the solution was stirred at 400 rpm open to air. The solution was heated to bath temperature 66° C. Once at temperature 1,3-dihexyl 2-(6-bromohexyl) propanedioate (4.00 g, 9.186 mmol, 1 equiv.) in EtOH (18.372 mL, 0.5 M) was added dropwise via addition funnel. After complete addition the solution was allowed to stir at 66° C., 400 rpm, under air atmosphere. The reaction was monitored by LCMS. After 24 h the reaction was done. Turned off the heat and let the solution cool to room temperature. Solvent was removed in vacuo and dissolved the crude in 300 mL MTBE. Washed the organic layer with satd NaHCO₃aq and brine. Back extracted the aqueous layer with 200 mL MTBE. Combined the organic layers and dried over Na₂SO₄, filtered, and concentrated in vacuo. Crude material purified via flash column chromatography; 220 g silica gel, 0-20% MeOH in DCM with 1% NH₄OH. Collected fractions a56-b26 yielding 1,3-dihexyl 2-[6-({3-[(tert-butoxycarbonyl)amino]propyl}amino)hexyl]propanedioate (1.804 g, 3.412 mmol, 37.14% yield) as a yellow oil. LCMS and HNMR supports structure to be desired product. CAD purity was 84%. UPLC/ELSD: RT=12.811 min. MS (EZ): m/z (MH⁺) 529.53. ¹H NMR (300 MHz, CDCl₃) δ 4.09 (td, J=6.7, 2.1 Hz, 4H), 3.29 (t, J=7.5 Hz, 1H), 3.17 (q, J=6.3 Hz, 2H), 2.63 (t, J=6.6 Hz, 2H), 2.54 (t, J=7.1 Hz, 2H), 1.86 (q, J=7.2 Hz, 2H), 1.69-1.52 (m, 7H), 1.41 (s, 11H), 1.39-1.21 (m, 19H), 0.91-0.81 (m, 6H).

### tridecan-7-yl 8-bromooctanoate

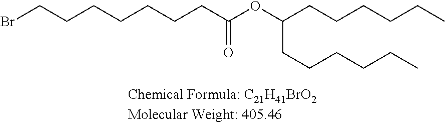 [SMILES: CCCCCCC(CCCCCC)OC(=O)CCCCCCCBr]

For procedure see 1,3-dihexyl 2-{[(8-bromooctanoyl)oxy]methyl}-2-methylpropanedioate. ¹H NMR (300 MHz, CDCl₃) δ 4.87 (p, J=6.2 Hz, 1H), 3.40 (t, J=6.8 Hz, 2H), 2.28 (t, J=7.4 Hz, 2H), 1.93-1.71 (m, 2H), 1.70-1.57 (m, 2H), 1.50 (h, J=5.7 Hz, 6H), 1.44-1.22 (m, 21H), 0.93-0.82 (m, 6H).

### Compound M11: 1,3-dihexyl 2-{6-[(3-{[2-(methylamino)-3,4-dioxocyclobut-1-en-1-yl]amino}propyl)[8-oxo-8-(pentadecan-8-yloxy)octyl]amino]hexyl}propanedioate

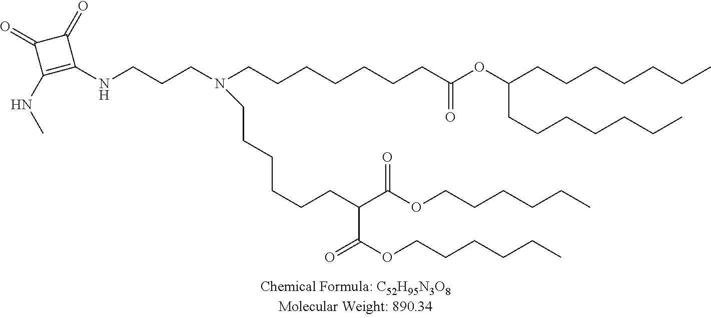 [SMILES: CCCCCCCC(CCCCCCC)OC(=O)CCCCCCCN(CCCCCCC(C(=O)OCCCCCC)C(=O)OCCCCCC)CCCNc1c(NC)c(=O)c1=O]

For procedure see Compound M3: Dihexyl 2-(6-((8-(heptadecan-9-yloxy)-8-oxooctyl)(3-((2-(methylamino)-3,4-dioxocyclobut-1-en-1-yl)amino)propyl)amino)hexyl)malonate. UPLC/ELSD: RT=13.975 min. MS(ES): m/z (MH⁺) 891.22. ¹H NMR (300 MHz, CD₃OD) δ 4.12 (td, J=6.5, 5.3 Hz, 4H), 3.61 (s, 1H), 3.35 (s, 12H), 3.25 (s, 3H), 2.50 (dt, J=27.7, 7.6 Hz, 6H), 2.31 (t, J=7.2 Hz, 2H), 1.87-1.71 (m, 4H), 1.68-1.44 (m, 13H), 1.42-1.25 (m, 42H), 1.15 (d, J=6.2 Hz, 3H), 0.91 (td, J=6.0, 3.9 Hz, 12H).

### 1,3-dihexyl 2-{6-[(3-aminopropyl)[8-oxo-8-(pentadecan-8-yloxy) octyl]amino]hexyl}propanedioate

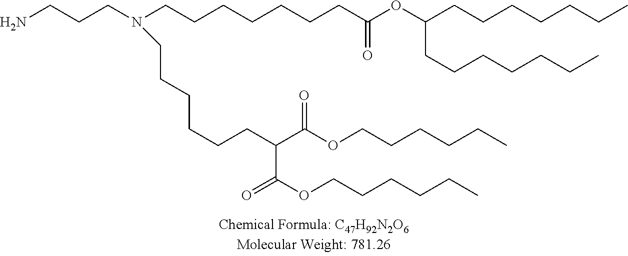 [SMILES: CCCCCCCC(CCCCCCC)OC(=O)CCCCCCCN(CCCN)CCCCCCC(C(=O)OCCCCCC)C(=O)OCCCCCC]

For procedure see Dihexyl 2-(6-((3-aminopropyl) (8-(heptadecan-9-yloxy)-8-oxooctyl)amino)hexyl)malonate. MS(ES): m/z (M+) 781.95. ¹H NMR (300 MHz, CD₃OD) δ 5.49 (s, 4H), 4.22-4.03 (m, 4H), 2.52 (dt, J=27.2, 7.4 Hz, 6H), 1.75-1.58 (m, 8H), 1.45-1.26 (m, 44H), 0.97-0.85 (m, 12H).

### 1,3-dihexyl 2-[6-({3-[(tert-butoxycarbonyl)amino]propyl}[8-oxo-8-(pentadecan-8-yloxy)octyl]amino)hexyl]propanedioate

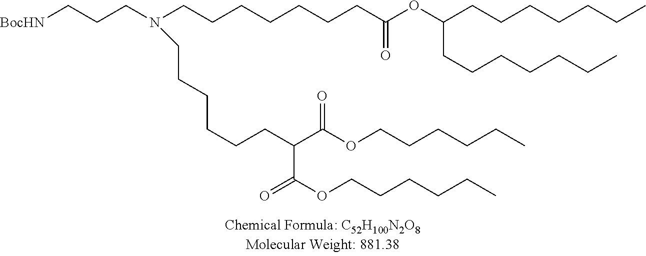 [SMILES: CCCCCCCC(CCCCCCC)OC(=O)CCCCCCCN(CCCCCCC(C(=O)OCCCCCC)C(=O)OCCCCCC)CCCNC(=O)OC(C)(C)C]

For procedure see Dihexyl 2-(6-((3-((tert-butoxycarbonyl)amino)propyl)(8-(heptadecan-9-yloxy)-8-oxooctyl)amino)hexyl)malonate. UPLC/ELSD RT=14.028 min. MS ES: m/z (MH⁺) 882.09. ¹H NMR (300 MHz, CDCl₃) δ 4.28-3.91 (m, 4H), 3.03 (dd, J=15.2, 8.5 Hz, 2H), 2.44 (dt, J=15.5, 7.6 Hz, 6H), 2.27 (t, J=7.2 Hz, 2H), 1.83 (t, J=7.4 Hz, 2H), 1.40 (s, 10H), 1.33-1.23 (m, 39H), 0.87 (td, J=6.7, 4.6 Hz, 12H).

### pentadecan-8-yl 8-bromooctanoate

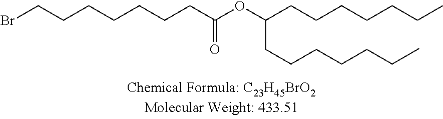 [SMILES: CCCCCCCC(CCCCCCC)OC(=O)CCCCCCCBr]

For procedure see 1,3-dihexyl 2-{[(8-bromooctanoyl)oxy]methyl}-2-methylpropanedioate. ¹H NMR (300 MHz, CDCl₃) δ 4.86 (p, J=6.2 Hz, 1H), 3.39 (t, J=6.8 Hz, 2H), 2.28 (t, J=7.4 Hz, 2H), 1.92-1.71 (m, 2H), 1.70-1.55 (m, 3H), 1.55-1.38 (m, 7H), 1.38-1.16 (m, 26H), 0.96 (d, J=6.6 Hz, 1H), 0.87 (td, J=6.6, 2.0 Hz, 8H).

### Compound M9: 1,3-dipentyl 2-(6-{[8-(heptadecan-9-yloxy)-8-oxooctyl](3-{[2-(methylamino)-3,4-dioxocyclobut-1-en-1-yl]amino}propyl)amino}hexyl)propanedioate

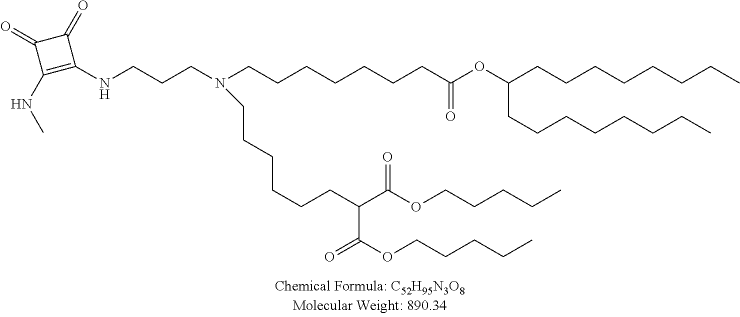 [SMILES: CCCCCCCCC(CCCCCCCC)OC(=O)CCCCCCCN(CCCCCCC(C(=O)OCCCCC)C(=O)OCCCCC)CCCNc1c(NC)c(=O)c1=O]

For procedure see Compound M3: Dihexyl 2-(6-((8-(heptadecan-9-yloxy)-8-oxooctyl) (3-((2-(methylamino)-3,4-dioxocyclobut-1-en-1-yl)amino)propyl)amino)hexyl)malonate. UPLC/ELSD: RT=9.083 min. MS(ES): m/z (MH⁺) 891.96. ¹H NMR (300 MHz, CDCl₃) δ 4.86 (q, J=6.2 Hz, 1H), 4.12 (td, J=6.7, 2.4 Hz, 4H), 3.67 (s, 1H), 3.49 (s, 12H), 3.32 (t, J=7.4 Hz, 1H), 3.27 (d, J=4.8 Hz, 3H), 2.64 (s, 2H), 2.49 (s, 3H), 2.28 (t, J=7.4 Hz, 2H), 1.62 (q, J=6.6 Hz, 9H), 1.33 (dt, J=9.5, 4.1 Hz, 25H), 1.25 (s, 17H), 1.05 (s, 1H), 0.89 (q, J=7.0 Hz, 13H).

### 1,3-dipentyl 2-{6-[(3-aminopropyl)[8-(heptadecan-9-yloxy)-8-oxooctyl]amino]hexyl}propanedioate

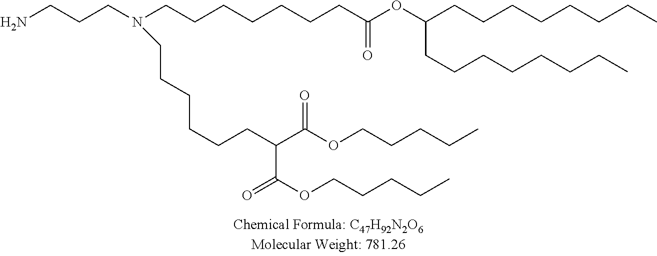 [SMILES: CCCCCCCCC(CCCCCCCC)OC(=O)CCCCCCCN(CCCN)CCCCCCC(C(=O)OCCCCC)C(=O)OCCCCC]

For procedure see Dihexyl 2-(6-((3-aminopropyl) (8-(heptadecan-9-yloxy)-8-oxooctyl)amino)hexyl)malonate. MS(ES): m/z (MH⁺) 782.07. ¹H NMR (300 MHz, CD₃OD) δ 4.12 (q, J=6.6 Hz, 4H), 3.31-2.99 (m, 12H), 2.32 (t, J=7.3 Hz, 2H), 2.17-2.01 (m, 2H), 1.86 (q, J=7.5 Hz, 2H), 1.75-1.59 (m, 10H), 1.57 (s, 5H), 1.53 (d, J=5.9 Hz, 3H), 1.44-1.34 (m, 17H), 1.32 (s, 10H), 1.29 (s, 16H), 0.91 (q, J=6.9 Hz, 12H).

### 1,3-dibutyl 2-[6-({3-[(tert-butoxycarbonyl)amino]propyl}[8-(heptadecan-9-yloxy)-8-oxooctyl]amino)hexyl]propanedioate

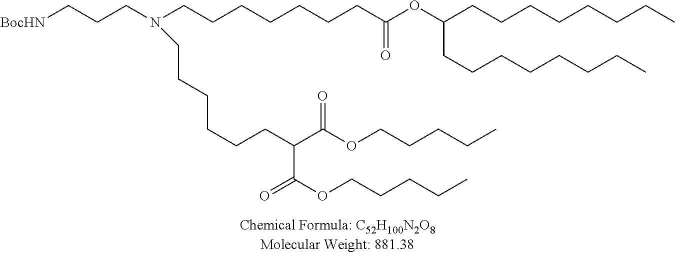 [SMILES: CCCCCCCCC(CCCCCCCC)OC(=O)CCCCCCCN(CCCCCCC(C(=O)OCCCCC)C(=O)OCCCCC)CCCNC(=O)OC(C)(C)C]

For procedure see Dihexyl 2-(6-((3-((tert-butoxycarbonyl)amino)propyl) (8-(heptadecan-9-yloxy)-8-oxooctyl)amino)hexyl)malonate. UPLC/ELSD: RT=10.028 min. MS(ES): m/z (MH⁺) 882.21. ¹H NMR (300 MHz, CDCl₃) δ 5.63 (s, 1H), 4.86 (p, J=6.2 Hz, 1H), 4.11 (td, J=6.7, 2.2 Hz, 4H), 3.31 (t, J=7.5 Hz, 1H), 3.16 (q, J=6.0 Hz, 2H), 2.48-2.21 (m, 8H), 1.88 (q, J=7.4 Hz, 2H), 1.67 (d, J=11.4 Hz, 3H), 1.64-1.55 (m, 7H), 1.50 (h, J=5.7 Hz, 5H), 1.43 (s, 11H), 1.37-1.22 (m, 44H), 0.88 (q, J=6.9 Hz, 12H).

### 1,3-dipentyl 2-(6-bromohexyl)propanedioate

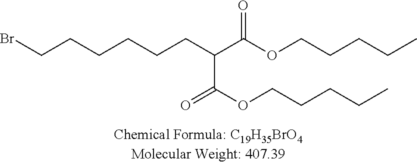 [SMILES: CCCCCOC(=O)C(CCCCCCBr)C(=O)OCCCCC]

For procedure see Dioctyl 2-(6-bromohexyl)malonate. ¹H NMR (300 MHz, CDCl₃) δ 4.10 (q, J=6.6 Hz, 4H), 3.35 (dt, J=22.9, 7.1 Hz, 3H), 1.84 (ddt, J=15.2, 8.9, 5.8 Hz, 5H), 1.61 (h, J=6.9 Hz, 5H), 1.51-1.31 (m, 10H), 1.29 (d, J=3.9 Hz, 5H), 1.15 (ddt, J=15.3, 12.1, 5.6 Hz, 2H), 0.94-0.84 (m, 6H).

### 1,3-dipentyl propanedioate

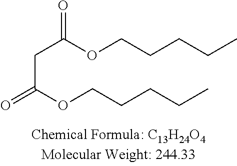 [SMILES: CCCCCOC(=O)CC(=O)OCCCCC]

For procedure see 1,3-dihexyl 2-(hydroxymethyl)-2-methylpropanedioate. ¹H NMR (300 MHz, CDCl₃) δ 4.12 (td, J=6.8, 1.1 Hz, 4H), 3.35 (d, J=1.0 Hz, 2H), 1.73-1.56 (m, 5H), 1.44-1.27 (m, 9H), 1.25 (d, J=3.0 Hz, 1H), 0.99-0.79 (m, 8H).

### Compound M10: 1,3-dibutyl 2-(6-{[8-(heptadecan-9-yloxy)-8-oxooctyl](3-{[2-(methylamino)-3,4-dioxocyclobut-1-en-1-yl]amino}propyl)amino}hexyl)propanedioate

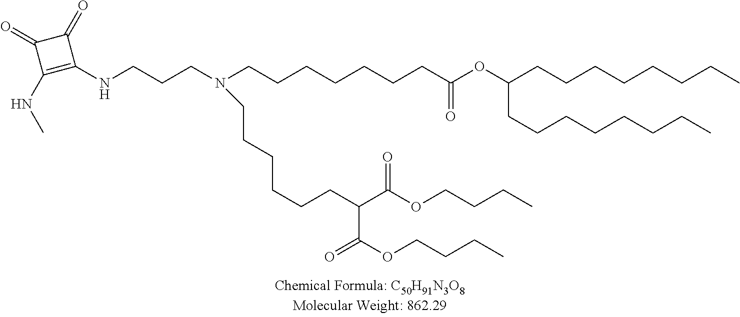 [SMILES: CCCCCCCCC(CCCCCCCC)OC(=O)CCCCCCCN(CCCCCCC(C(=O)OCCCC)C(=O)OCCCC)CCCNc1c(NC)c(=O)c1=O]

For procedure see Compound M3: Dihexyl 2-(6-((8-(heptadecan-9-yloxy)-8-oxooctyl) (3-((2-(methylamino)-3,4-dioxocyclobut-1-en-1-yl)amino)propyl)amino)hexyl)malonate. UPLC/ELSD RT=7.483 min. MS(ES): m/z (MH⁺) 863.10. ¹H NMR (300 MHz, CDCl₃) δ 4.85 (p, J=6.1 Hz, 1H), 4.13 (td, J=6.6, 2.6 Hz, 4H), 3.65 (s, 1H), 3.49 (d, J=3.0 Hz, 10H), 3.38-3.22 (m, 4H), 2.53 (t, J=5.7 Hz, 2H), 2.40 (t, J=7.6 Hz, 4H), 2.29 (t, J=7.4 Hz, 2H), 1.88 (d, J=7.1 Hz, 2H), 1.74 (t, J=6.0 Hz, 2H), 1.67-1.54 (m, 13H), 1.49 (s, OH), 1.46-1.36 (m, 7H), 1.28 (d, J=17.3 Hz, 34H), 1.00 (s, 3H), 0.97-0.82 (m, 12H).

### 1,3-dibutyl 2-{6-[(3-aminopropyl) [8-(heptadecan-9-yloxy)-8-oxooctyl]amino]hexyl}propanedioate

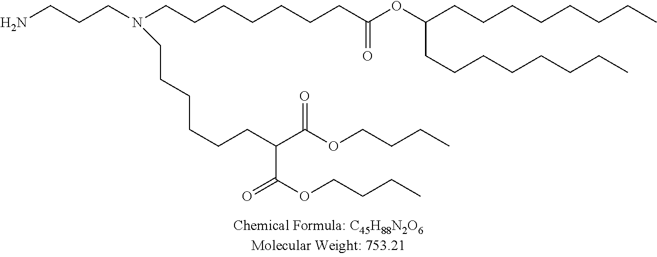 [SMILES: CCCCCCCCC(CCCCCCCC)OC(=O)CCCCCCCN(CCCN)CCCCCCC(C(=O)OCCCC)C(=O)OCCCC]

For procedure see Dihexyl 2-(6-((3-aminopropyl) (8-(heptadecan-9-yloxy)-8-oxooctyl)amino)hexyl)malonate. MS(ES): m/z (M+) 753.95. ¹H NMR (300 MHz, CD₃OD) δ 4.21-4.05 (m, 4H), 3.21 (dt, J=29.8, 8.3 Hz, 10H), 3.10-2.99 (m, 3H), 2.32 (t, J=7.3 Hz, 2H), 2.15-2.01 (m, 2H), 1.86 (q, J=7.4 Hz, 2H), 1.71 (s, 3H), 1.69-1.49 (m, 14H), 1.39 (d, J=7.8 Hz, 19H), 1.29 (s, 25H), 1.00-0.85 (m, 12H).

### 1,3-dibutyl 2-[6-({3-[(tert-butoxycarbonyl)amino]propyl}[8-(heptadecan-9-yloxy)-8-oxooctyl]amino)hexyl]propanedioate

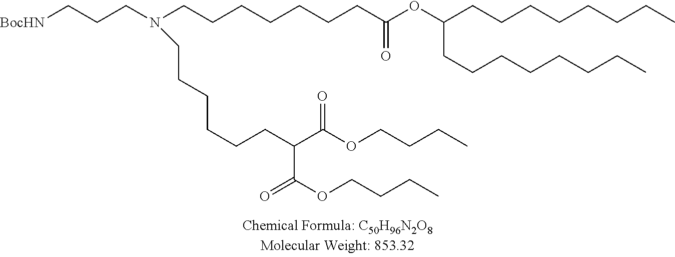 [SMILES: CCCCCCCCC(CCCCCCCC)OC(=O)CCCCCCCN(CCCCCCC(C(=O)OCCCC)C(=O)OCCCC)CCCNC(=O)OC(C)(C)C]

For procedure see Dihexyl 2-(6-((3-((tert-butoxycarbonyl)amino)propyl) (8-(heptadecan-9-yloxy)-8-oxooctyl)amino)hexyl)malonate. UPLC/ELSD RT=8.783 min. MS(ES): m/z (MH⁺) 854.09. ¹H NMR (300 MHz, CDCl₃) δ 5.63 (s, 1H), 4.86 (p, J=6.2 Hz, 1H), 4.12 (td, J=6.6, 2.6 Hz, 4H), 3.31 (t, J=7.5 Hz, 1H), 3.16 (q, J=6.1 Hz, 2H), 2.48-2.35 (m, 3H), 2.35-2.21 (m, 5H), 1.88 (q, J=7.4 Hz, 2H), 1.69-1.53 (m, 9H), 1.49 (d, J=6.0 Hz, 4H), 1.43 (s, 11H), 1.40-1.32 (m, 9H), 1.32-1.27 (m, 11H), 1.25 (s, 22H), 0.97-0.82 (m, 12H).

### 1,3-dibutyl 2-(6-bromohexyl) propanedioate

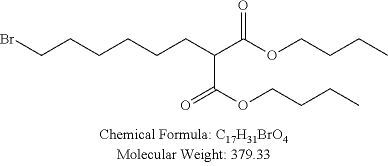 [SMILES: CCCCOC(=O)C(CCCCCCBr)C(=O)OCCCC]

For procedure see Dioctyl 2-(6-bromohexyl)malonate. ¹H NMR (300 MHz, CDCl₃) δ 4.13 (td, J=6.6, 2.4 Hz, 5H), 3.39 (t, J=6.8 Hz, 2H), 3.31 (t, J=7.5 Hz, 1H), 1.95-1.76 (m, 4H), 1.69-1.54 (m, 6H), 1.49-1.22 (m, 12H), 0.92 (t, J=7.3 Hz, 8H).

propanedioic acid, dibutyl ester

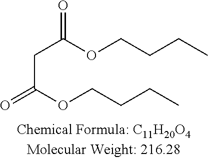

For procedure see 1,3-dihexyl 2-(hydroxymethyl)-2-methylpropanedioate. ¹H NMR (300 MHz, CDCl₃) δ 4.13 (t, J=6.6 Hz, 4H), 3.34 (s, 2H), 1.73-1.53 (m, 4H), 1.45-1.27 (m, 4H), 0.91 (t, J=7.3 Hz, 6H).

### Example 2: LNP Formulation and In Vivo Protein Expression

LNPs comprising ionizable lipids described herein were formulated according to Table 2 and the following general procedure. Properties of the resulting LNPs including N: P, size, PDI, % EE are also shown in Table 2.

General procedure for one-pot LNP formation: Lipid stock solutions in ethanol are mixed with mRNA in 25 mM acetate solution at a 3:1 ratio in a microfluidic mixer. The resulting LNPs, which are at approximately pH 5 and 25% EtOH, are buffer exchanged by dialysis into the storage buffer. The LNP solutions are then concentrated by Amicon® and purified by a 0.2 uM sterile filter.

TABLE 2
LNP Formulations and Properties

| | | | | | | | | Total | | Endo- | | | |
|---|---|---|---|---|---|---|---|---|---|---|---|---|---|
| | | | | | | | | [mRNA] | | toxin | | Osmolality | |
| | | | Mol % | | | Size | | in | % | (EU/ | | (mOsm/ | Storage |
| Grp | Lipid | Composition | Ratio | mRNA | N:P | (nm) | PDI | TX | EE | mL) | pH | kg) | Buffer |
| 1 | L4 | L4: | 48:2: | m10 | 4.9 | 104.5 | 0.328 | 193 | 92.6 | | | 311 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 2 | L5 | L5: | 48:2: | m10 | 3.4 | 60.4 | 0.117 | 284 | 95.2 | | | 313 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 3 | M1 | M1: | 48:2: | m10 | 4.9 | 57.0 | 0.164 | 298 | 96.9 | | | 314 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 4 | M3 | M3: | 48:2: | m10 | 4.9 | 59.3 | 0.148 | 288 | 97.8 | | | 317 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 5 | M2 | M2: | 48:2: | m10 | 4.9 | 58.0 | 0.186 | 346 | 97.9 | | | 315 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 6 | L1 | L1: | 48:2: | m10 | 4.9 | 64.4 | 0.197 | 310 | 96.8 | | | 319 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 7 | L2 | L2: | 48:2: | m10 | 4.9 | 60.7 | 0.177 | 227 | 95.8 | | | 315 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 8 | L3 | L3: | 48:2: | m10 | 4.9 | 54.4 | 0.172 | 251 | 95.9 | | | 317 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 1A | L4 | L4: | 48:2: | m10 | 4.9 | 74.9 | 0.130 | 375 | 94.8 | 2.3 | 7.444 | 317 | 20 mM |
| | | PEG-1: | 11:39 | hEPO | | | | | | | | | Tris 8% |
| | | DSPC: | | miR 142 | | | | | | | | | Sucrose, |
| | | Chol | | miR 126 | | | | | | | | | pH 7.4 |
| 2A | L5 | L5: | 48:2: | m10 | 3.4 | 59.9 | 0.119 | 369 | 96.1 | 2 | 7.456 | 320 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 3A | L6 | L6: | 48:2: | m10 | 4.9 | 58.2 | 0.166 | 347 | 96.9 | 2.3 | 7.468 | 321 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 4A | M1 | M1: | 48:2: | m10 | 4.9 | 58.7 | 0.128 | 388 | 94.1 | 2.4 | 7.452 | 320 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 5A | M3 | M3: | 48:2: | m10 | 4.9 | 59.1 | 0.132 | 356 | 98.3 | 2 | 7.155 | 318 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 6A | M4 | M4: | 48:2: | m10 | 4.9 | 68.7 | 0.099 | 327 | 92.2 | 2.6 | 7.450 | 321 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 7A | M5 | M5: | 48:2: | m10 | 4.9 | 126.7 | 0.496 | 312 | 69.8 | 2.7 | 6.962 | 320 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |
| 8A | M6 | M6: | 48:2: | m10 | 4.9 | 104.9 | 0.418 | 312 | 67.8 | <2 | 7.450 | 317 | 20 mM |
| | | PEG-1: | 11:39 | hEPO | | | | | | | | | Tris 8% |
| | | DSPC: | | miR 142 | | | | | | | | | Sucrose, |
| | | Cholesterol | | miR 126 | | | | | | | | | pH 7.4 |

The LNPs were tested in vivo (systemic administration) for hEPO protein expression. See Table 3A and Table 3B. As shown, ionizable lipids with malonate tails form stable LNPs with desirable biophysical properties, leading to effective protein expression in vivo.

TABLE 3A
hEPO Protein Expression In Vivo

| | | 6 hr protein | 24 hr protein |
|---|---|---|---|
| | | expression | expression |
| Group | Ionizable Lipid | ratio cf. L5 | ratio cf. L5 |
| 1 | L4 | 0.1 | 0.1 |
| 2 | L5 | 1.0 | 1.0 |
| 3 | M1 | 0.9 | 0.9 |
| 4 | M3 | 1.0 | 1.2 |
| 5 | M2 | 0.3 | 0.3 |
| 6 | L1 | 0.1 | 0.1 |
| 7 | L2 | 0.7 | 0.8 |
| 8 | L3 | 0.6 | 0.6 |

NT = Not Tested

TABLE 3B
hEPO Protein Expression and In Vivo

| | | 6 hr protein | 24 hr protein |
|---|---|---|---|
| | | expression | expression |
| Group | Ionizable Lipid | ratio cf. L5 | ratio cf. L5 |
| 1A | L4 | 0.08 | 0.07 |
| 2A | L5 | 1.00 | 1.00 |
| 3A | L6 | 0.94 | 0.84 |
| 4A | M1 | 1.70 | 1.46 |
| 5A | M3 | 2.53 | 2.42 |
| 6A | M4 | 0.34 | 0.36 |
| 7A | M5 | 1.37 | 1.15 |
| 8A | M6 | 0.82 | 0.81 |
| Tris-sucrose | N/A | N/A | N/A |

NT = Not Tested

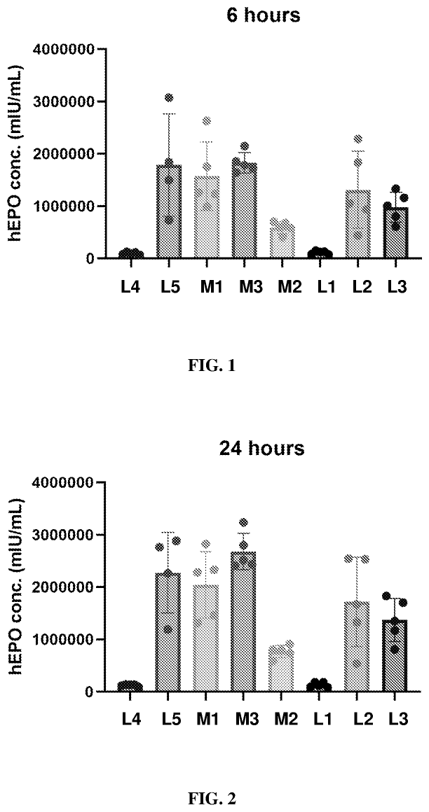

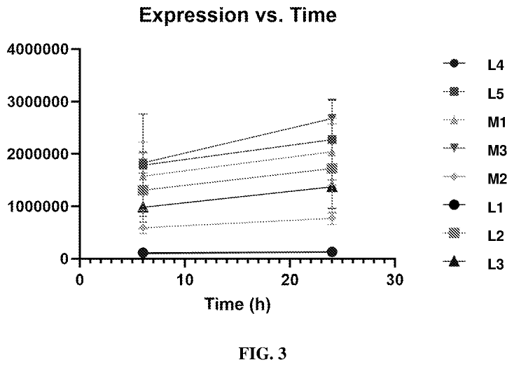

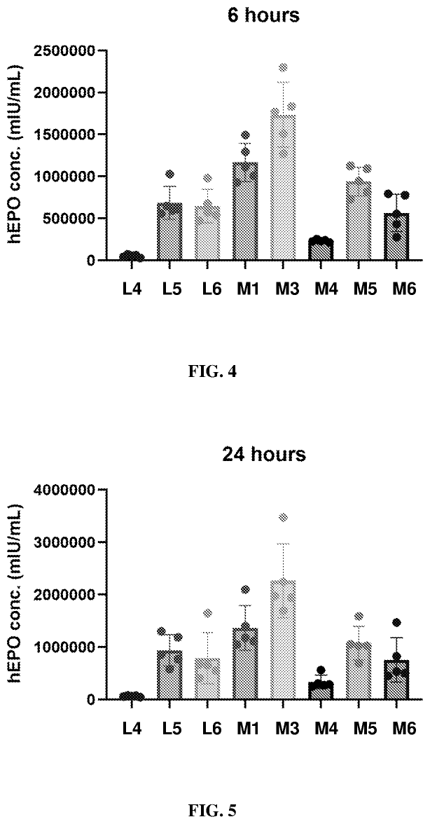

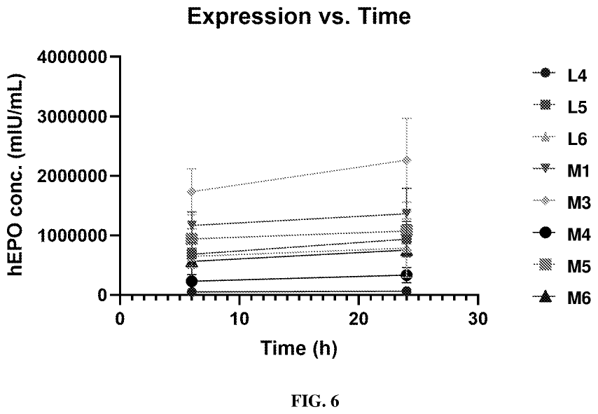

See also FIGS. 1-6. FIG. 1 shows in vivo hEPO protein expression at 6 hours with LNPs formulated with ionizable lipids M1-M3. FIG. 2 shows in vivo hEPO protein expression at 24 hours with LNPs formulated with ionizable lipids M1-M3. FIG. 3 shows a comparison of the 6 hour and 24 hour timepoints. FIG. 4 shows in vivo hEPO protein expression at 6 hours with LNPs formulated with ionizable lipids M1 and M3-M6. FIG. 5 shows in vivo hEPO protein expression at 24 hours with LNPs formulated with ionizable lipids M1 and M3-M6. FIG. 6 shows a comparison of the 6 hour and 24 hour timepoints.

Certain lipids referenced in this study are as indicated below in Table 4.

TABLE 4

| | |
|---|---|
| L2 | 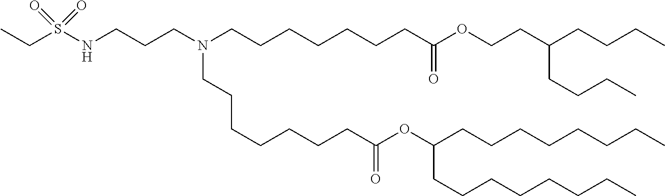 [SMILES: CCCCCCCCC(CCCCCCCC)OC(=O)CCCCCCCN(CCCCCCCC(=O)OCCC(CCCC)CCCC)CCCNS(=O)(=O)CC] |
| L4 | 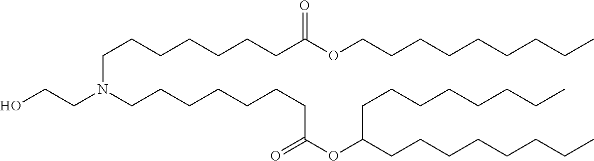 [SMILES: CCCCCCCCCOC(=O)CCCCCCCN(CCO)CCCCCCCC(=O)OC(CCCCCCCC)CCCCCCCC] |
| L5 | 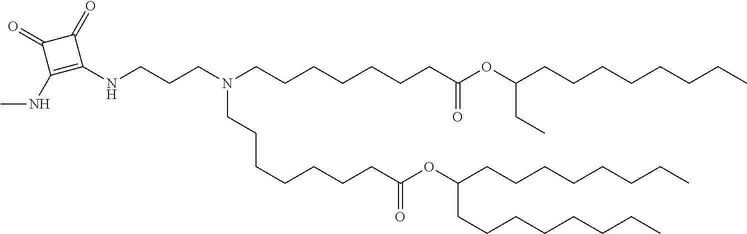 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| L6 | 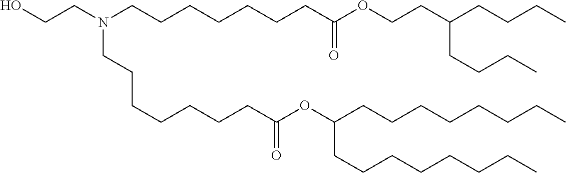 [SMILES: CCCCCCCCC(CCCCCCCC)OC(=O)CCCCCCCN(CCO)CCCCCCCC(=O)OCCC(CCCC)CCCC] |
| PEG-1 | 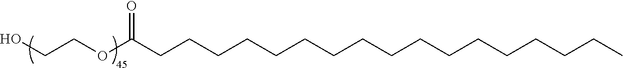 |

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I): 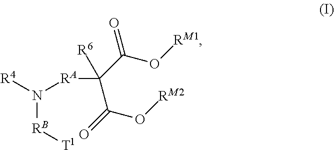
or a pharmaceutically acceptable salt thereof, wherein: 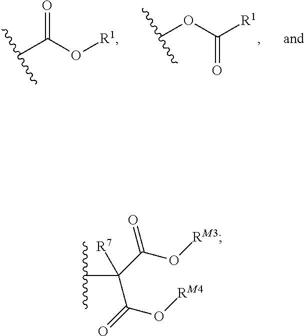
T¹ is selected from:
R^{A} and R^{B} are each independently optionally substituted C₄₋₂₀ alkylene, optionally substituted C₄₋₂₀ alkenylene, or optionally substituted C₄₋₂₀ alkynylene;
R¹ is optionally substituted, branched or unbranched C₁₋₃₀ alkyl, optionally substituted, branched or unbranched C₂₋₃₀ alkenyl, or optionally substituted, branched or unbranched C₂₋₃₀ alkynyl;
R^{M1}, R^{M2}, R^{M3}, and R^{M4} are each independently optionally substituted, branched or unbranched C₁₋₂₀ alkyl, optionally substituted, branched or unbranched C₂₋₂₀ alkenyl, or optionally substituted, branched or unbranched C₂₋₂₀ alkynyl;
R⁴ is a head group selected from optionally substituted C₁₋₆ alkyl, optionally substituted C₂₋₆ alkenyl, optionally substituted C₂₋₆ alkynyl, optionally substituted C₃₋₈ carbocyclyl, and —Y¹-Q;
Y¹ is optionally substituted C₁₋₁₀ alkylene, optionally substituted C₂₋₁₀ alkenylene, and optionally substituted C₂₋₁₀ alkynylene;
Q is optionally substituted C₃₋₈ carbocyclyl, optionally substituted C₆₋₁₀ aryl, optionally substituted C₃₋₈ heterocyclyl, optionally substituted C₅₋₁₀ heteroaryl, —OR^{O}, —O(CH₂)ₙN(R^{N})₂, —O(CH₂)ₙOR^{O}, —C(O)OR^{O}, —OC(O)R, —OC(O)OR^{O}, —CX₃, —CX₂H, —CXH₂, —CN, —N(R^{N})₂, —N(R^{N})(CH₂)ₙN(R^{N})₂, —N(R^{N})(CH₂)ₙOR^{O}, —C(O)N(R^{N})₂, —N(R^{N})C(O)R, —N(R^{N})C(O)N(R^{N})₂, —N(R^{N})C(S)N(R^{N})₂, —N(R^{N})C(═NR⁵)N(R^{N})₂, —N(R^{N})C(═CHR⁵)N(R^{N})₂, —C(═NR⁵)N(R^{N})₂, —C(═NR⁵)R, —OC(O)N(R^{N})₂, —N(R^{N})C(O)OR^{O}, —C(R)N(R^{N})₂C(O)OR^{O}, —N(R^{N})S(O)₂R, —N(R^{N})S(O)R, —S(O)₂N(R^{N})₂, —S(O)N(R^{N})₂, 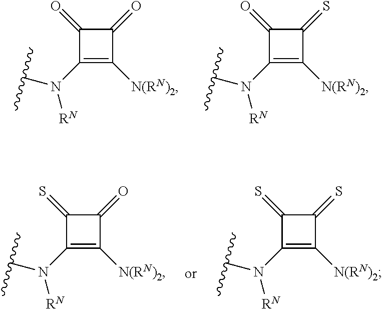
each R is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, or optionally substituted C₅₋₁₀ heteroaryl;
each R^{O} is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or an oxygen protecting group;
each R^{N} is independently hydrogen, —OR^{O}, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or a nitrogen protecting group, or two R^{N} attached to the same nitrogen atoms are joined together with the intervening atoms to form optionally substituted heterocyclyl or optionally substituted heteroaryl;
R⁵ is hydrogen, —CN, —NO₂, —OR^{O}, —S(O)₂R, —S(O)₂ (R^{N})₂, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, or optionally substituted C₁₋₆ acyl, or optionally a nitrogen protecting group when attached to a nitrogen atom;
R⁶ and R⁷ are each independently hydrogen, halogen, or optionally substituted C₁₋₆ alkyl;
each X is independently halogen; and
n is 1, 2, 3, 4, or 5.

2. A compound of Formula (I): 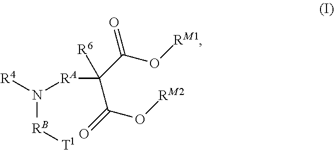
or a pharmaceutically acceptable salt thereof, wherein:
T¹ is selected from: 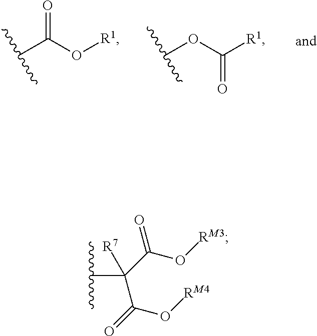
R^{A} and R^{B} are each independently optionally substituted C₁₋₂₀ alkylene, optionally substituted C₁₋₂₀ alkenylene, or optionally substituted C₁₋₂₀ alkynylene;
R¹ is optionally substituted, branched or unbranched C₁₋₃₀ alkyl, optionally substituted, branched or unbranched C₂₋₃₀ alkenyl, or optionally substituted, branched or unbranched C₂₋₃₀ alkynyl;
R^{M1}, R^{M2}, R^{M3}, and R^{M4} are each independently optionally substituted, branched or unbranched C₁₋₂₀ alkyl, optionally substituted, branched or unbranched C₂₋₂₀ alkenyl, or optionally substituted, branched or unbranched C₂₋₂₀ alkynyl;
R⁴ is a head group selected from optionally substituted C₁₋₆ alkyl, optionally substituted C₂₋₆ alkenyl, optionally substituted C₂₋₆ alkynyl, optionally substituted C₃₋₈ carbocyclyl, and —Y¹-Q;
Y¹ is optionally substituted C₁₋₁₀ alkylene, optionally substituted C₂₋₁₀ alkenylene, and optionally substituted C₂₋₁₀ alkynylene;
Q is optionally substituted C₃₋₈ carbocyclyl, optionally substituted C₆₋₁₀ aryl, optionally substituted C₃₋₈ heterocyclyl, optionally substituted C₅₋₁₀ heteroaryl, —OR^{O}, —O(CH₂)ₙN(R^{N})₂, —O(CH₂)ₙOR^{O}, —C(O)OR^{O}, —OC(O)R, —OC(O)OR^{O}, —CX₃, —CX₂H, —CXH₂, —CN, —N(R^{N})₂, —N(R^{N})(CH₂)ₙN(R^{N})₂, —N(R^{N})(CH₂)ₙOR^{O}, —C(O)N(R^{N})₂, —N(R^{N})C(O)R, —N(R^{N})C(O)N(R^{N})₂, —N(R^{N})C(S)N(R^{N})₂, —N(R^{N})C(═NR⁵)N(R^{N})₂, —N(R^{N})C(═CHR⁵)N(R^{N})₂, —C(═NR⁵)N(R^{N})₂, —C(═NR⁵)R, —OC(O)N(R^{N})₂, —N(R^{N})C(O)OR^{O}, —C(R)N(R^{N})₂C(O)OR^{O}, —N(R^{N})S(O)₂R, —N(R^{N})S(O)R, —S(O)₂N(R^{N})₂, —S(O)N(R^{N})₂, 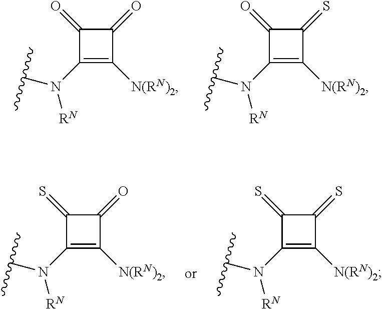
each R is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, or optionally substituted C₅₋₁₀ heteroaryl;
each R^{O} is independently hydrogen, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or an oxygen protecting group;
each R^{N} is independently hydrogen, —OR^{O}, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, optionally substituted C₅₋₁₀ heteroaryl, optionally substituted C₁₋₆ acyl, or a nitrogen protecting group, or two R^{N} attached to the same nitrogen atoms are joined together with the intervening atoms to form optionally substituted heterocyclyl or optionally substituted heteroaryl;
R⁵ is hydrogen, —CN, —NO₂, —OR^{O}, —S(O)₂R, —S(O)₂ (R^{N})₂, optionally substituted C₁₋₆ alkyl, optionally substituted C₂₋₆ alkenyl, optionally substituted C₂₋₆ alkynyl, optionally substituted C₃₋₈ carbocyclyl, optionally substituted C₆₋₁₀ aryl, optionally substituted C₃₋₈ heterocyclyl, or optionally substituted C₅₋₁₀ heteroaryl, or optionally substituted C₁₋₆ acyl, or optionally a nitrogen protecting group when attached to a nitrogen atom;
R⁶ and R⁷ are each independently hydrogen, halogen, or optionally substituted C₁₋₆ alkyl; each X is independently halogen; and
n is 1, 2, 3, 4, or 5;
provided that the total collective carbon atom content of the alkyl, alkylene, alkenyl, alkenylene, alkynyl, and alkynylene groups of R^{A}, R^{M1}, and R^{M2}, excluding optional substituents, is from 10-40 carbon atoms, inclusive; and/or
provided that the total collective carbon atom content of the alkyl, alkylene, alkenyl, alkenylene, alkynyl, and alkynylene groups of R^{B} and R¹, excluding optional substituents, is from 10-40 carbon atoms, inclusive.

3. The compound of claim 1 or 2, wherein the compound is of Formula (I-a): 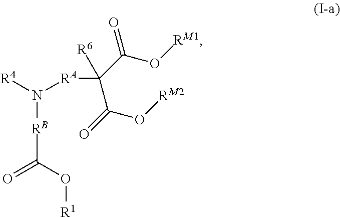
or a pharmaceutically acceptable salt thereof.

4. The compound of any one of claims 1-3, or a pharmaceutically acceptable salt thereof, wherein the total collective carbon atom content of the alkyl, alkylene, alkenyl, alkenylene, alkynyl, and alkynylene groups of R^{A}, R^{M1}, and R^{M2}, excluding optional substituents, is from 10-40 carbon atoms, inclusive.

5. The compound of any one of claims 1-3, or a pharmaceutically acceptable salt thereof, wherein the total collective carbon atom content of the alkyl, alkylene, alkenyl, alkenylene, alkynyl, and alkynylene groups of R^{A}, R^{M1}, and R^{M2}, excluding optional substituents, is from 10-30 carbon atoms, inclusive.

6. The compound of any one of claims 1-3, or a pharmaceutically acceptable salt thereof, wherein the total collective carbon atom content of the alkyl, alkylene, alkenyl, alkenylene, alkynyl, and alkynylene groups of R^{A}, R^{M1}, and R^{M2}, excluding optional substituents, is from 15-25 carbon atoms, inclusive.

7. The compound of any one of claims 1-6, or a pharmaceutically acceptable salt thereof, wherein the total collective carbon atom content of the alkyl, alkylene, alkenyl, alkenylene, alkynyl, and alkynylene groups of R^{B} and R¹, excluding optional substituents, is from 10-40 carbon atoms, inclusive.

8. The compound of any one of claims 1-6, or a pharmaceutically acceptable salt thereof, wherein the total collective carbon atom content of the alkyl, alkylene, alkenyl, alkenylene, alkynyl, and alkynylene groups of R^{B} and R¹, excluding optional substituents, is from 15-40 carbon atoms, inclusive.

9. The compound of any one of claims 1-6, or a pharmaceutically acceptable salt thereof, wherein the total collective carbon atom content of the alkyl, alkylene, alkenyl, alkenylene, alkynyl, and alkynylene groups of R^{B} and R¹, excluding optional substituents, is from 20-30 carbon atoms, inclusive.

10. The compound of any one of claims 1-9, or a pharmaceutically acceptable salt thereof, wherein R^{A} is optionally substituted C₄₋₂₀ alkylene.

11. The compound of any one of claims 1-10, or a pharmaceutically acceptable salt thereof, wherein R^{A} is optionally substituted C₄₋₁₀ alkylene.

12. The compound of any one of claims 1-11, or a pharmaceutically acceptable salt thereof, wherein R^{A} is unsubstituted C₄₋₁₀ alkylene.

13. The compound of any one of claims 1-12, or a pharmaceutically acceptable salt thereof, wherein R^{A} is:

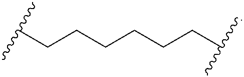 [SMILES: *CCCCCC*]

14. The compound of any one of claims 1-13, or a pharmaceutically acceptable salt thereof, wherein R^{B} is optionally substituted C₄₋₂₀ alkylene.

15. The compound of any one of claims 1-14, or a pharmaceutically acceptable salt thereof, wherein R^{B} is optionally substituted C₄₋₁₀ alkylene.

16. The compound of any one of claims 1-15, or a pharmaceutically acceptable salt thereof, wherein R^{B} is unsubstituted C₄₋₁₀ alkylene.

17. The compound of any one of claims 1-16, or a pharmaceutically acceptable salt thereof, wherein R^{B} is:

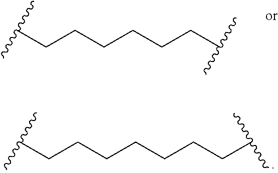 [SMILES: *CCCCCC* ; *CCCCCCC*]

18. The compound of claim 1 or 2, wherein the compound is of Formula (I-b): 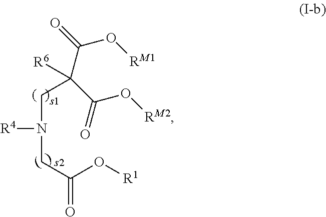
or a pharmaceutically acceptable salt thereof, wherein:
s1 and s2 are each independently an integer from 4-10, inclusive.

19. The compound of any one of claims 1-18, or a pharmaceutically acceptable salt thereof, wherein R^{M1} is optionally substituted, branched or unbranched C₁₋₂₀ alkyl.

20. The compound of any one of claims 1-19, or a pharmaceutically acceptable salt thereof, wherein R^{M1} is optionally substituted, branched or unbranched C₁₋₁₀ alkyl.

21. The compound of any one of claims 1-20, or a pharmaceutically acceptable salt thereof, wherein R^{M1} is unsubstituted, unbranched C₁₋₁₀ alkyl.

22. The compound of any one of claims 1-21, or a pharmaceutically acceptable salt thereof, wherein R^{M1} is:

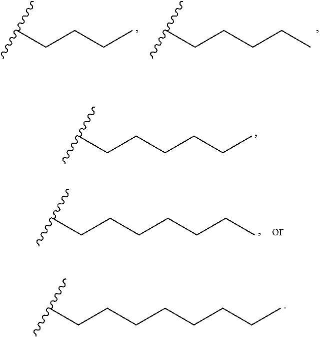

23. The compound of any one of claims 1-22, or a pharmaceutically acceptable salt thereof, wherein R^{M2} is optionally substituted, branched or unbranched C₁₋₂₀ alkyl.

24. The compound of any one of claims 1-23, or a pharmaceutically acceptable salt thereof, wherein R^{M2} is optionally substituted, branched or unbranched C₁₋₁₀ alkylene.

25. The compound of any one of claims 1-24, or a pharmaceutically acceptable salt thereof, wherein R^{M2} is unsubstituted, unbranched C₁₋₁₀ alkyl.

26. The compound of any one of claims 1-25, or a pharmaceutically acceptable salt thereof, wherein R^{M2} is:

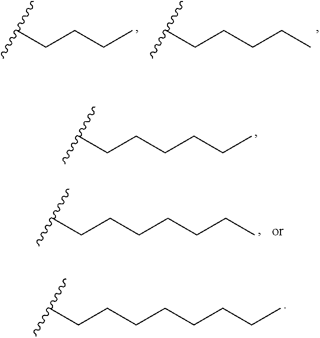

27. The compound of any one of claims 1, 2, 4-6, 10-17, and 19-26, or a pharmaceutically acceptable salt thereof, wherein R^{M3} is optionally substituted, branched or unbranched C₁₋₂₀ alkyl.

28. The compound of any one of claims 1, 2, 4-6, 10-17, and 19-27, or a pharmaceutically acceptable salt thereof, wherein R^{M3} is optionally substituted, branched or unbranched C₁₋₁₀ alkyl.

29. The compound of any one of claims 1, 2, 4-6, 10-17, and 19-28, or a pharmaceutically acceptable salt thereof, wherein R^{M3} is unsubstituted, unbranched C₁₋₁₀ alkyl.

30. The compound of any one of claims 1, 2, 4-6, 10-17, and 19-29, or a pharmaceutically acceptable salt thereof, wherein R^{M3} is:

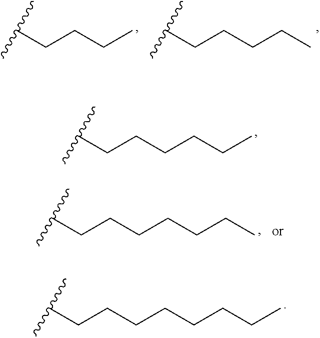

31. The compound of any one of claims 1, 2, 4-6, 10-17, and 19-30, or a pharmaceutically acceptable salt thereof, wherein R^{M4} is optionally substituted, branched or unbranched C₁₋₂₀ alkyl.

32. The compound of any one of claims 1, 2, 4-6, 10-17, and 19-31, or a pharmaceutically acceptable salt thereof, wherein R^{M4} is optionally substituted, branched or unbranched C₁₋₁₀ alkylene.

33. The compound of any one of claims 1, 2, 4-6, 10-17, and 19-32, or a pharmaceutically acceptable salt thereof, wherein R^{M4} is unsubstituted, unbranched C₁₋₁₀ alkyl.

34. The compound of any one of claims 1, 2, 4-6, 10-17, and 19-33, or a pharmaceutically acceptable salt thereof, wherein R^{M4} is:

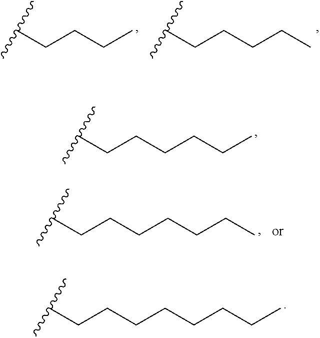

35. The compound of claim 18, wherein the compound is of Formula (I-c): 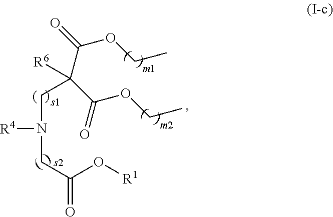
or a pharmaceutically acceptable salt thereof, wherein:
m1 and m2 are each independently 0 or an integer from 1-10, inclusive.

36. The compound of any one of claims 1-35, or a pharmaceutically acceptable salt thereof, wherein R⁴ is —Y¹-Q.

37. The compound of any one of claims 1-36, or a pharmaceutically acceptable salt thereof, wherein Y¹ is optionally substituted C₁₋₁₀ alkylene.

38. The compound of any one of claims 1-37, or a pharmaceutically acceptable salt thereof, wherein Y¹ is optionally substituted C₁₋₄ alkylene.

39. The compound of any one of claims 1-38, or a pharmaceutically acceptable salt thereof, wherein Y¹ is unsubstituted C₁₋₄ alkylene.

40. The compound of any one of claims 1-39, or a pharmaceutically acceptable salt thereof, wherein Y¹ is:

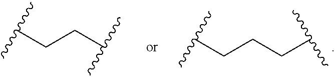

41. The compound of claim 35, wherein the compound is of Formula (I-d): 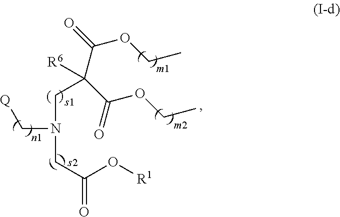
or a pharmaceutically acceptable salt thereof, wherein:
n1 is an integer from 1-10, inclusive.

42. The compound of any one of claims 1-26 and 35-41, or a pharmaceutically acceptable salt thereof, wherein R¹ is optionally substituted, branched or unbranched C₁₋₃₀ alkyl.

43. The compound of any one of claims 1-26 and 35-42, or a pharmaceutically acceptable salt thereof, wherein R¹ is optionally substituted, branched or unbranched C₁₀₋₃₀ alkyl.

44. The compound of any one of claims 1-26 and 35-43, or a pharmaceutically acceptable salt thereof, wherein R¹ is unsubstituted, branched C₁₀₋₃₀ alkyl.

45. The compound of any one of claims 1-26 and 35-41, or a pharmaceutically acceptable salt thereof, wherein R¹ is of the formula: 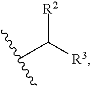
wherein R² and R³ are each independently optionally substituted, branched or unbranched C₁₋₂₀ alkyl, optionally substituted, branched or unbranched C₂₋₂₀ alkenyl, or optionally substituted, branched or unbranched C₂₋₂₀ alkynyl.

46. The compound of claim 45, wherein the compound is of Formula (I-e): 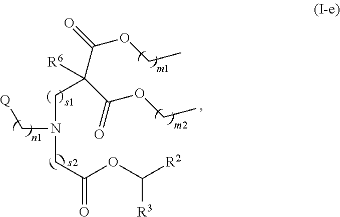
or a pharmaceutically acceptable salt thereof.

47. The compound of claim 45 or 46, or a pharmaceutically acceptable salt thereof, wherein R² is optionally substituted, branched or unbranched C₁₋₂₀ alkyl.

48. The compound of any one of claims 45-47, or a pharmaceutically acceptable salt thereof, wherein R² is optionally substituted, branched or unbranched C₁₋₁₀ alkyl.

49. The compound of any one of claims 45-48, or a pharmaceutically acceptable salt thereof, wherein R² is unsubstituted, unbranched C₁₋₁₀ alkyl.

50. The compound of any one of claims 45-49, or a pharmaceutically acceptable salt thereof, wherein R² is:

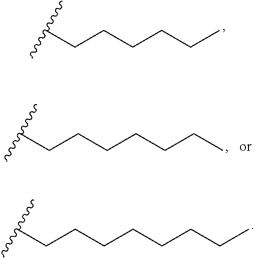

51. The compound of any one of claims 45-50, or a pharmaceutically acceptable salt thereof, wherein R³ is optionally substituted, branched or unbranched C₁₋₂₀ alkyl.

52. The compound of any one of claims 45-51, or a pharmaceutically acceptable salt thereof, wherein R³ is optionally substituted, branched or unbranched C₁₋₁₀ alkyl.

53. The compound of any one of claims 45-52, or a pharmaceutically acceptable salt thereof, wherein R³ is unsubstituted, unbranched C₁₋₁₀ alkyl.

54. The compound of any one of claims 45-53, or a pharmaceutically acceptable salt thereof, wherein R³ is:

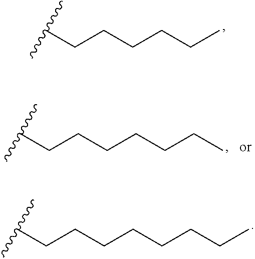

55. The compound of any one of claims 1-26 and 35-54, or a pharmaceutically acceptable salt thereof, wherein R¹ is:

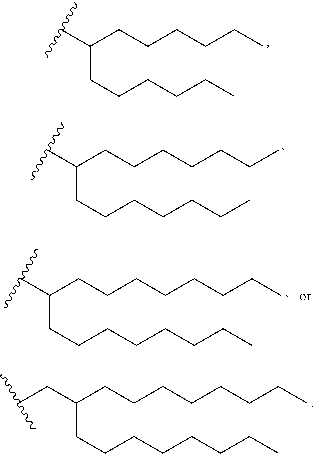 [SMILES: *C(CCCCCC)CCCCCC ; *C(CCCCCCC)CCCCCCC ; *C(CCCCCCCC)CCCCCCCC ; *CC(CCCCCCCC)CCCCCCCC]

56. The compound of claim 46, wherein the compound is of Formula (I-f): 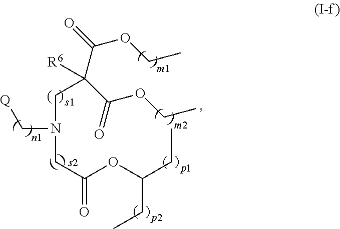
or a pharmaceutically acceptable salt thereof, wherein:
p1 and p2 are each independently 0 or an integer from 1-10, inclusive.

57. The compound of any one of claims 18-56, or a pharmaceutically acceptable salt thereof, wherein s1 is 6.

58. The compound of any one of claims 18-57, or a pharmaceutically acceptable salt thereof, wherein s2 is 6 or 7.

59. The compound of any one of claims 35-58, or a pharmaceutically acceptable salt thereof, wherein m1 is 3, 4, 5, 6, or 7.

60. The compound of any one of claims 35-59, or a pharmaceutically acceptable salt thereof, wherein m2 is 3, 4, 5, 6, or 7.

61. The compound of any one of claims 41-60, or a pharmaceutically acceptable salt thereof, wherein n1 is 2 or 3.

62. The compound of any one of claims 56-61, or a pharmaceutically acceptable salt thereof, wherein p1 is 5, 6, or 7.

63. The compound of any one of claims 56-62, or a pharmaceutically acceptable salt thereof, wherein p2 is 5, 6, or 7.

64. The compound of any one of claims 1-63, or a pharmaceutically acceptable salt thereof, wherein R⁶ is hydrogen, —CH₂OH, or —F.

65. The compound of any one of claims 1-64, or a pharmaceutically acceptable salt thereof, wherein R⁶ is hydrogen.

66. The compound of any one of claims 1, 2, 4-6, 10-17, 19-34, 36-40, 64, and 65, or a pharmaceutically acceptable salt thereof, wherein R⁷ is hydrogen.

67. The compound of any one of claims 1-66, or a pharmaceutically acceptable salt thereof, wherein Q is —OR^{O}, —N(R^{N})C(O)R, or

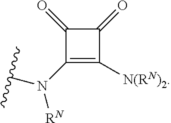

68. The compound of any one of claims 1-67, or a pharmaceutically acceptable salt thereof, wherein Q is —OH, —NHC(O) Me, —NHC(O) Et,

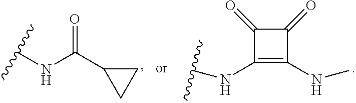

69. The compound of any one of claims 1-68, or a pharmaceutically acceptable salt thereof, wherein R⁴ is a head group selected from:

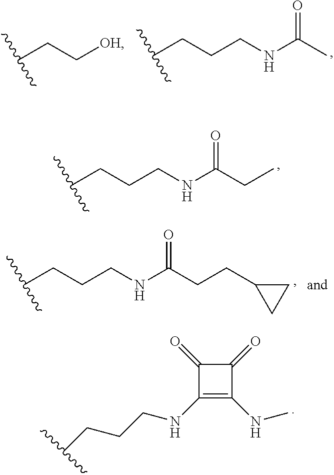

70. The compound of any one of claims 1-66, or a pharmaceutically acceptable salt thereof, wherein R⁴ is a head group selected from:

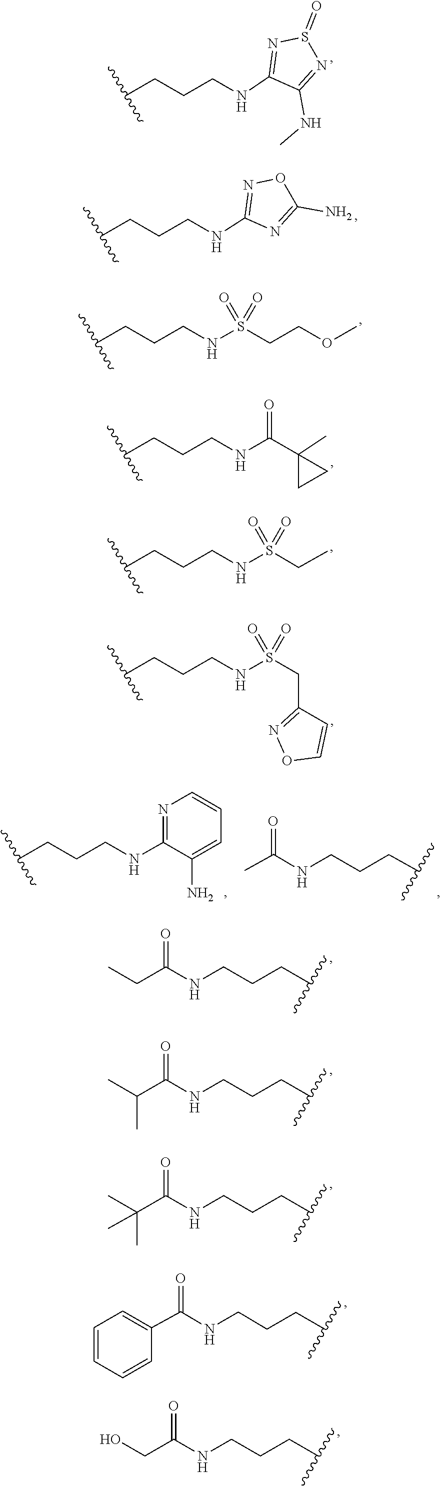

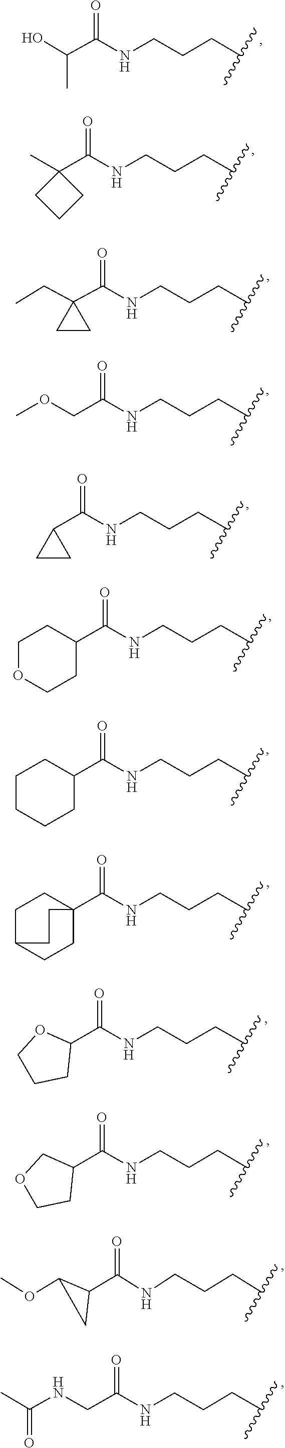

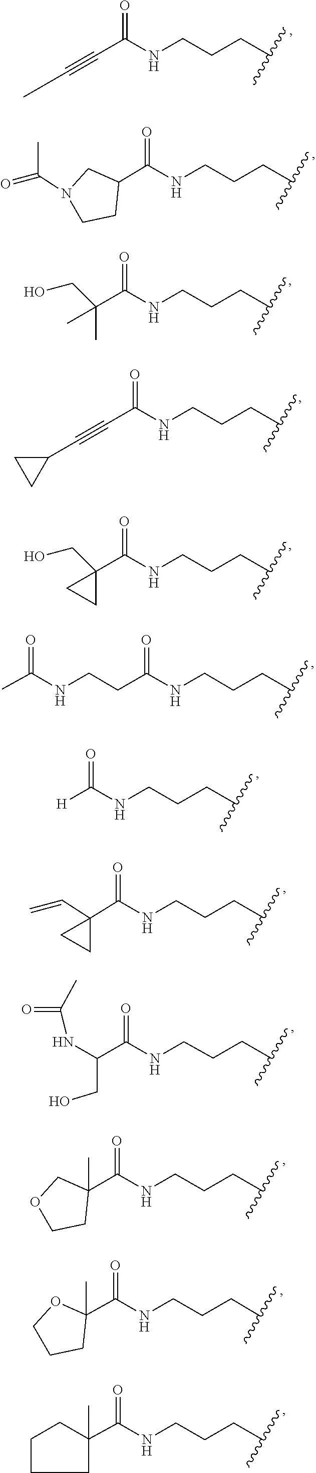

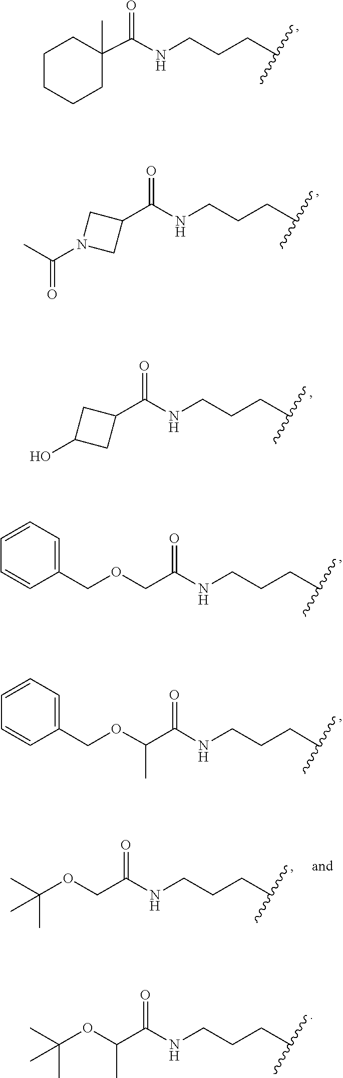

71. The compound of any one of claims 1-66, or a pharmaceutically acceptable salt thereof, wherein R⁴ is a head group selected from:

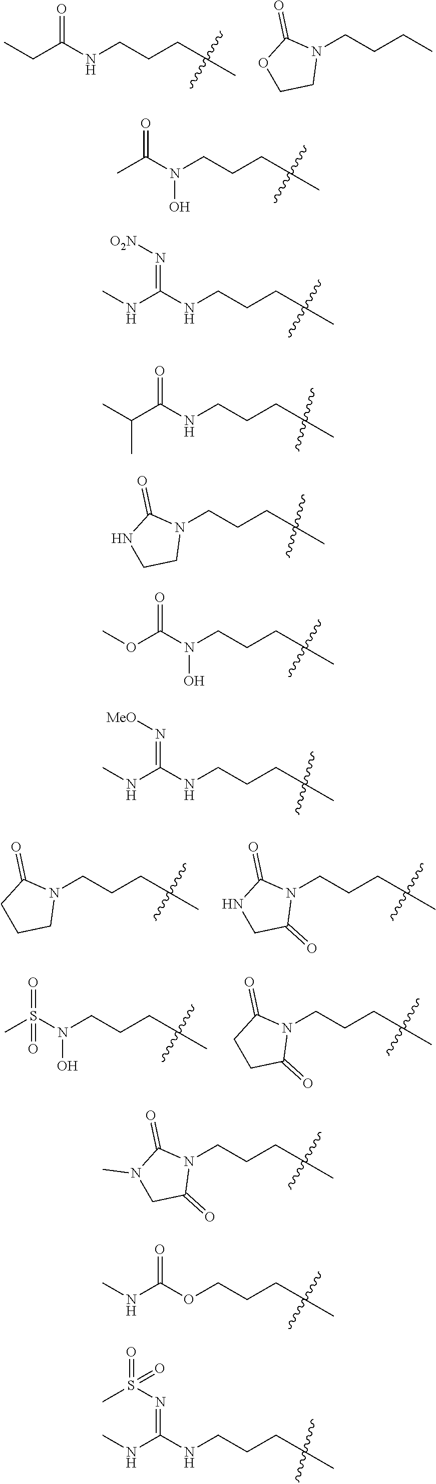

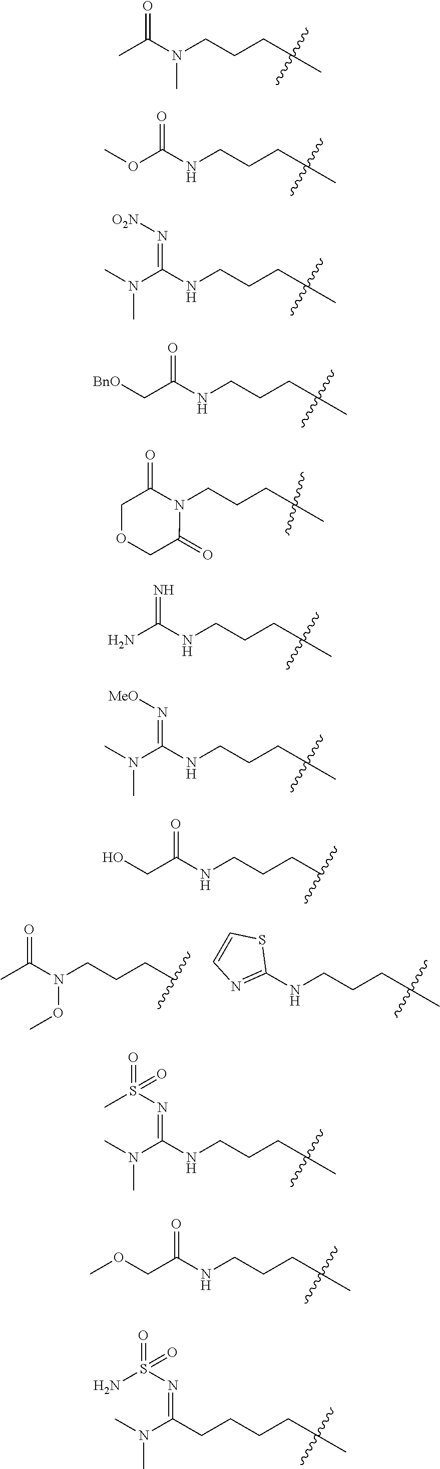

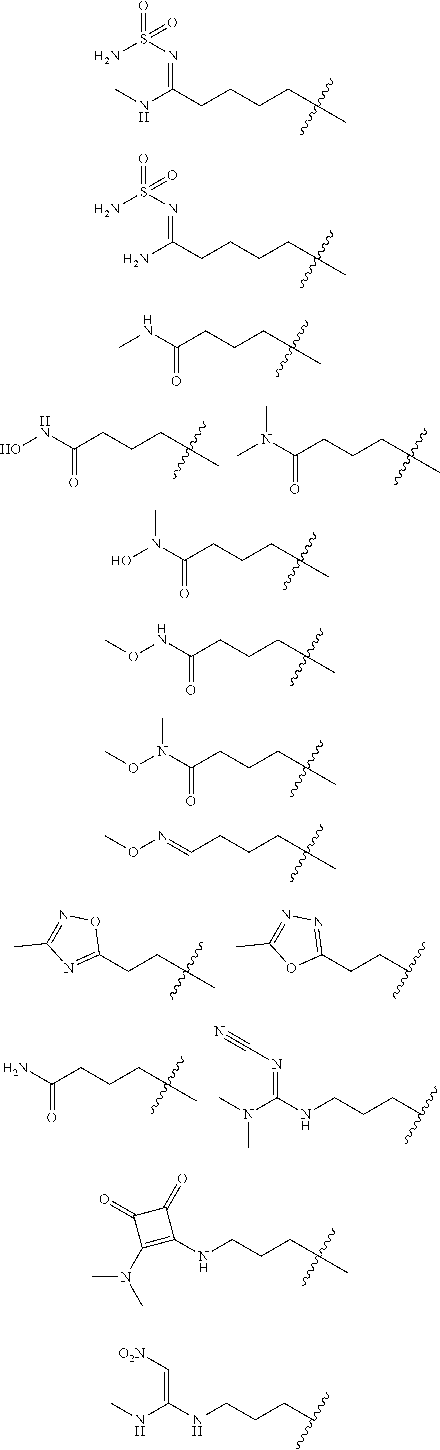

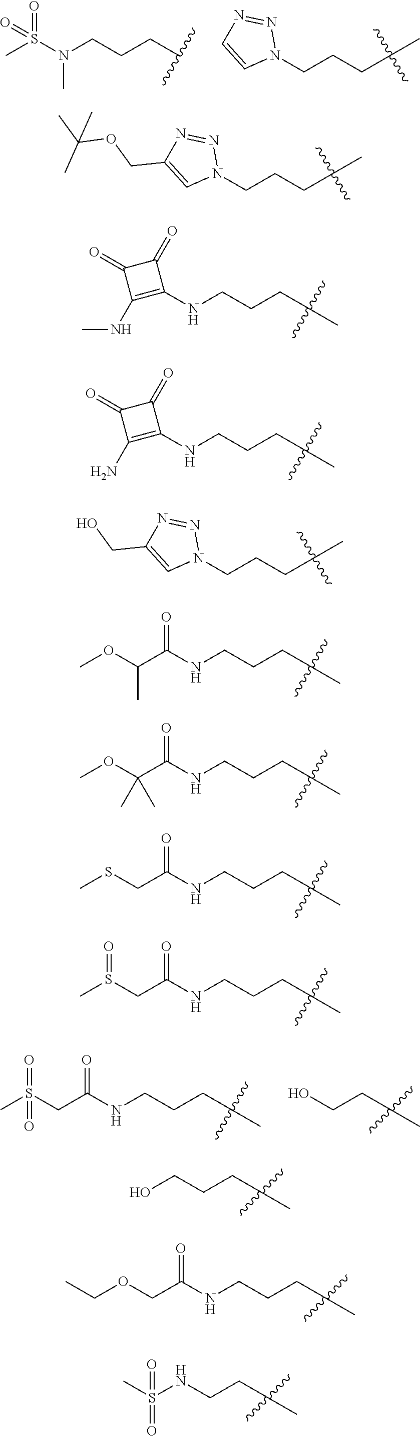

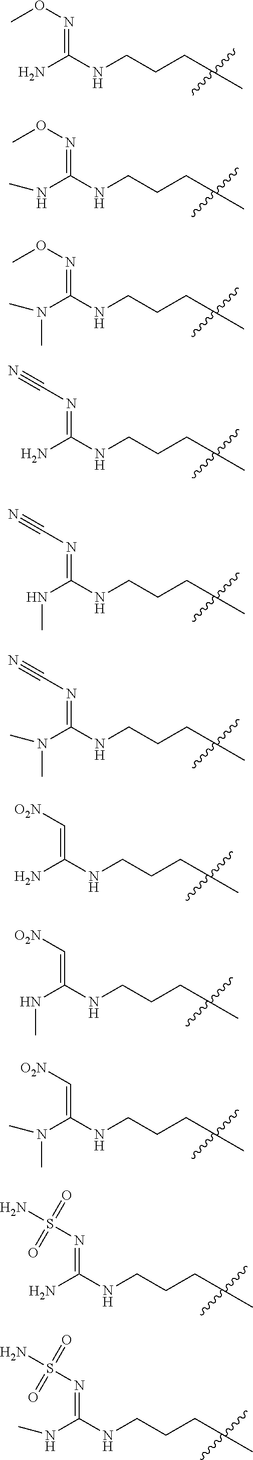

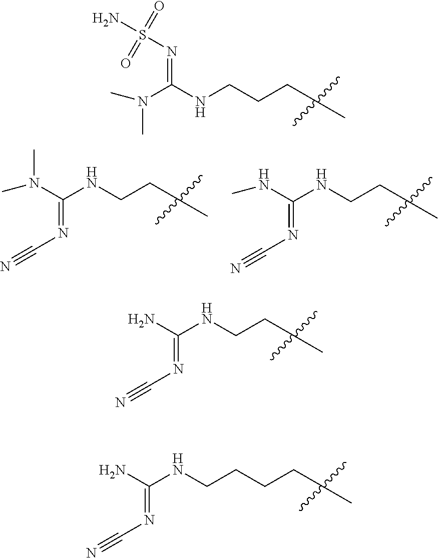

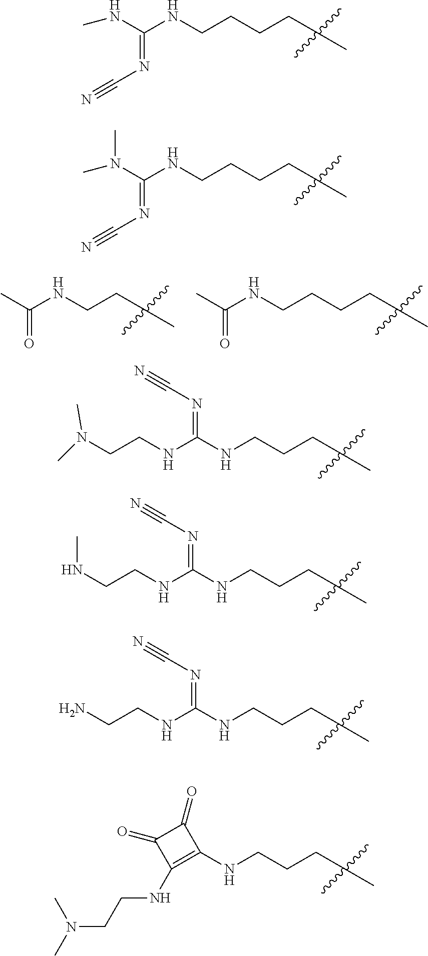

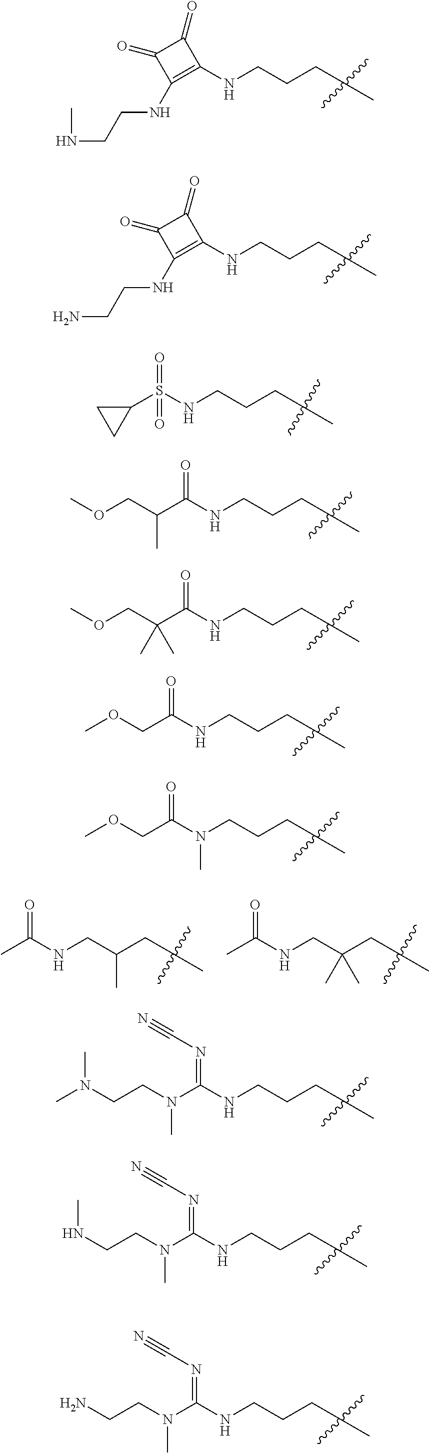

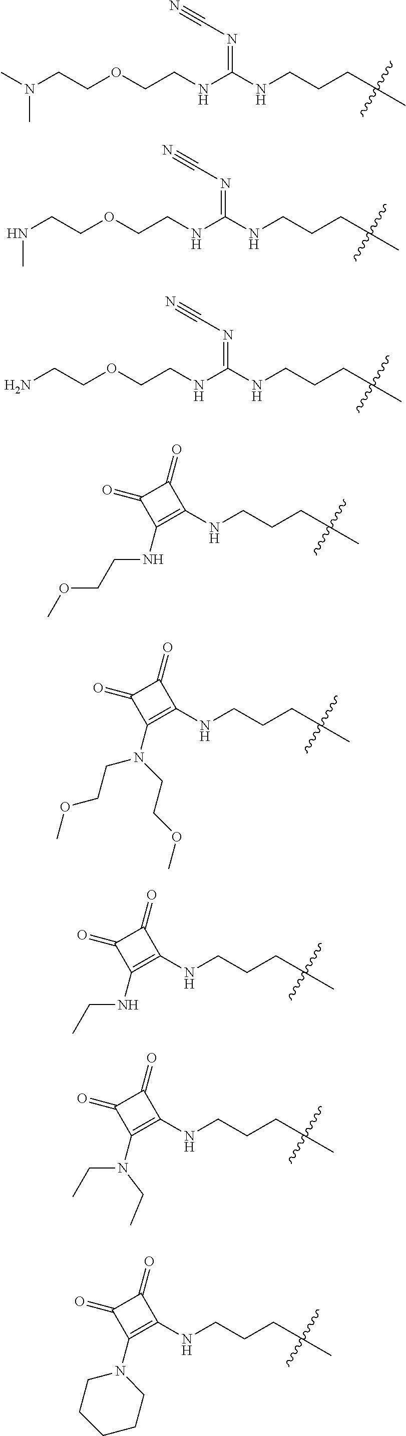

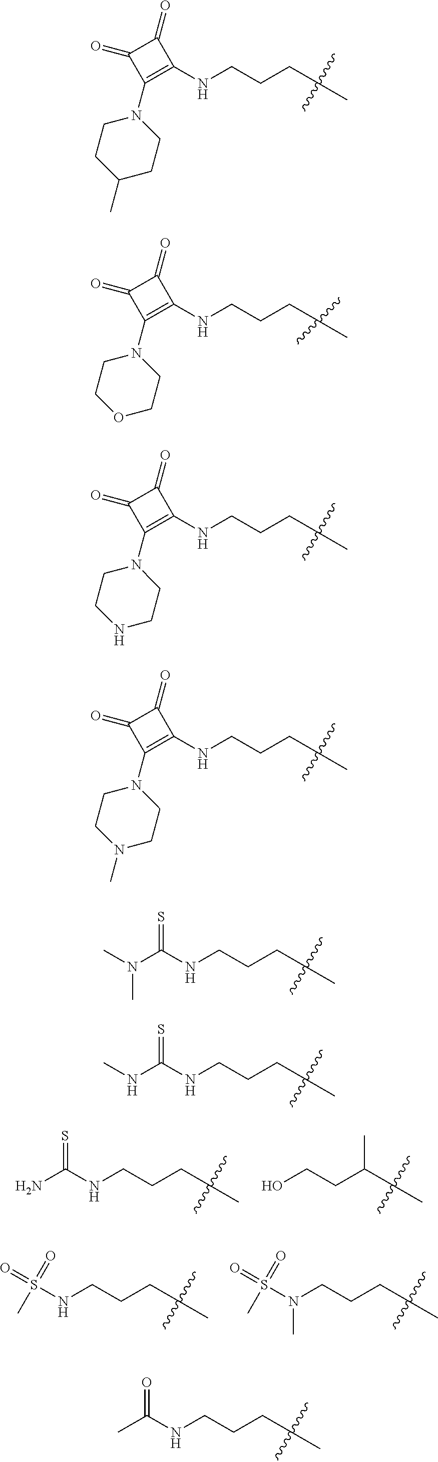

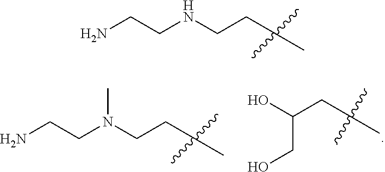

72. The compound of any one of the preceding claims, where the compounds is selected from one of the following:

| Compound | |
|---|---|
| No. | Structure |
| M1 | 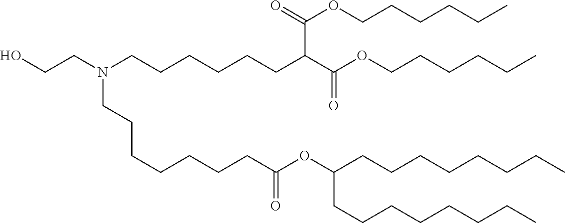 [SMILES: CCCCCCCCC(CCCCCCCC)OC(=O)CCCCCCCN(CCO)CCCCCCC(C(=O)OCCCCCC)C(=O)OCCCCCC] |
| M2 | 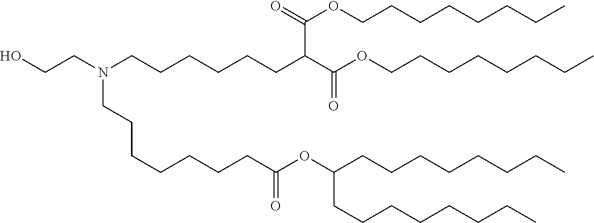 [SMILES: CCCCCCCCOC(=O)C(CCCCCCN(CCO)CCCCCCCC(=O)OC(CCCCCCCC)CCCCCCCC)C(=O)OCCCCCCCC] |
| M3 | 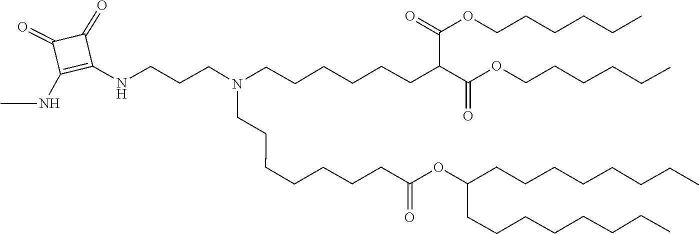 [SMILES: CCCCCCCCC(CCCCCCCC)OC(=O)CCCCCCCN(CCCCCCC(C(=O)OCCCCCC)C(=O)OCCCCCC)CCCNc1c(NC)c(=O)c1=O] |
| M4 | 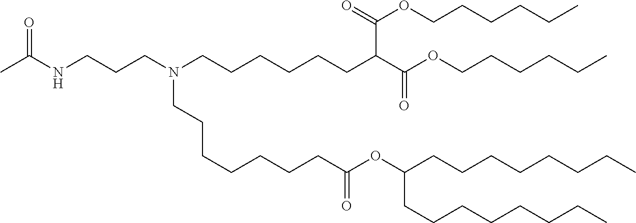 [SMILES: CCCCCCCCC(CCCCCCCC)OC(=O)CCCCCCCN(CCCCCCC(C(=O)OCCCCCC)C(=O)OCCCCCC)CCCNC(C)=O] |
| M5 | 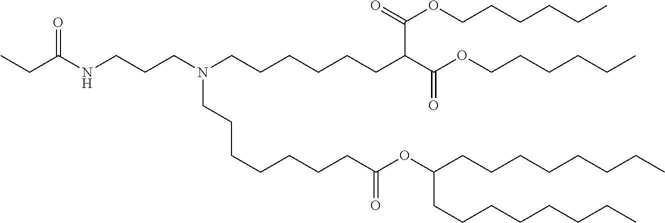 [SMILES: CCCCCCCCC(CCCCCCCC)OC(=O)CCCCCCCN(CCCCCCC(C(=O)OCCCCCC)C(=O)OCCCCCC)CCCNC(=O)CC] |
| M6 | 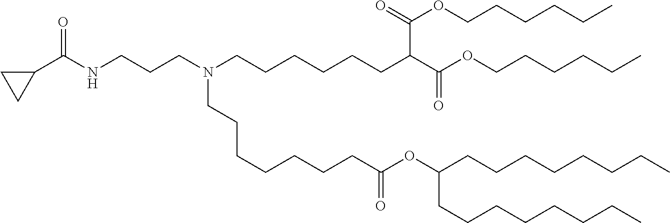 [SMILES: CCCCCCCCC(CCCCCCCC)OC(=O)CCCCCCCN(CCCCCCC(C(=O)OCCCCCC)C(=O)OCCCCCC)CCCNC(=O)C1CC1] |
| M7 | 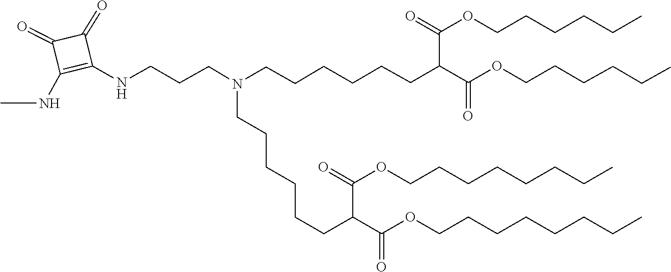 [SMILES: CCCCCCCCOC(=O)C(CCCCCCN(CCCCCCC(C(=O)OCCCCCC)C(=O)OCCCCCC)CCCNc1c(NC)c(=O)c1=O)C(=O)OCCCCCCCC] |
| M8 | 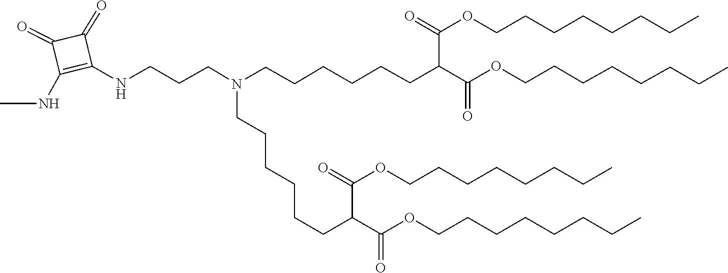 [SMILES: CCCCCCCCOC(=O)C(CCCCCCN(CCCCCCC(C(=O)OCCCCCCCC)C(=O)OCCCCCCCC)CCCNc1c(NC)c(=O)c1=O)C(=O)OCCCCCCCC] |
| M9 | 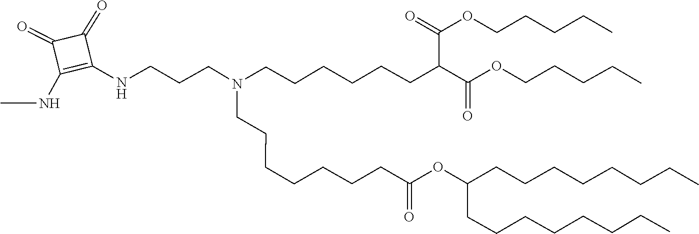 [SMILES: CCCCCCCCC(CCCCCCCC)OC(=O)CCCCCCCN(CCCCCCC(C(=O)OCCCCC)C(=O)OCCCCC)CCCNc1c(NC)c(=O)c1=O] |
| M10 | 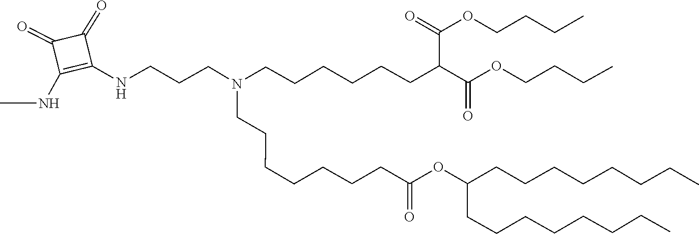 [SMILES: CCCCCCCCC(CCCCCCCC)OC(=O)CCCCCCCN(CCCCCCC(C(=O)OCCCC)C(=O)OCCCC)CCCNc1c(NC)c(=O)c1=O] |
| M11 | 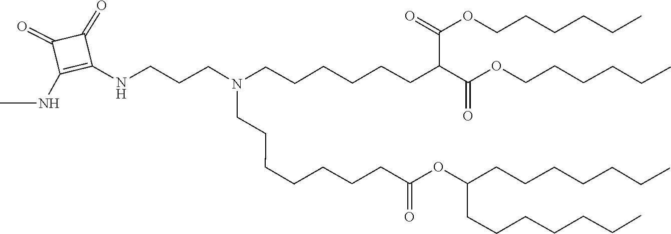 [SMILES: CCCCCCCC(CCCCCCC)OC(=O)CCCCCCCN(CCCCCCC(C(=O)OCCCCCC)C(=O)OCCCCCC)CCCNc1c(NC)c(=O)c1=O] |
| M12 | 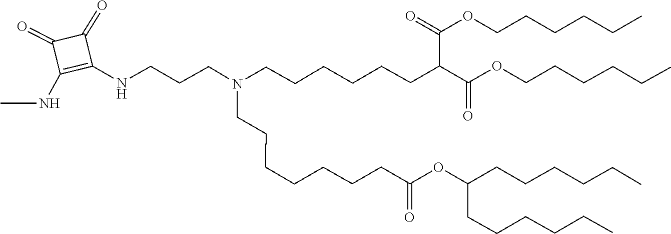 [SMILES: CCCCCCOC(=O)C(CCCCCCN(CCCCCCCC(=O)OC(CCCCCC)CCCCCC)CCCNc1c(NC)c(=O)c1=O)C(=O)OCCCCCC] |
| M13 | 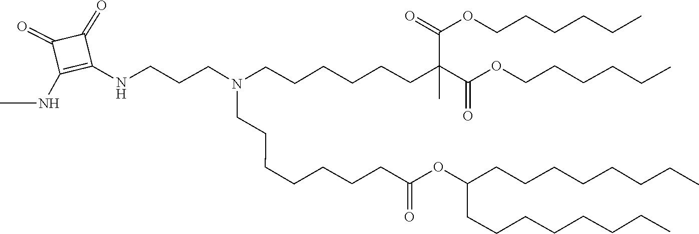 [SMILES: CCCCCCCCC(CCCCCCCC)OC(=O)CCCCCCCN(CCCCCCC(C)(C(=O)OCCCCCC)C(=O)OCCCCCC)CCCNc1c(NC)c(=O)c1=O] |
| M14 | 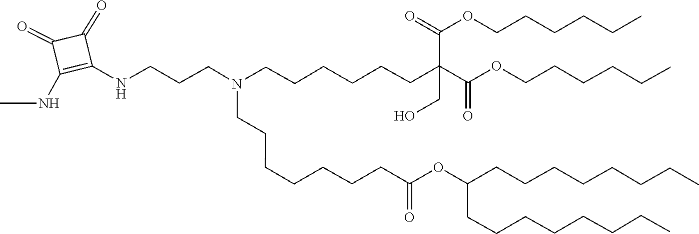 [SMILES: CCCCCCCCC(CCCCCCCC)OC(=O)CCCCCCCN(CCCCCCC(CO)(C(=O)OCCCCCC)C(=O)OCCCCCC)CCCNc1c(NC)c(=O)c1=O] |
| M15 | 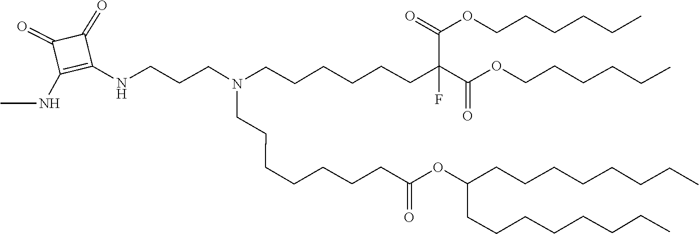 [SMILES: CCCCCCCCC(CCCCCCCC)OC(=O)CCCCCCCN(CCCCCCC(F)(C(=O)OCCCCCC)C(=O)OCCCCCC)CCCNc1c(NC)c(=O)c1=O] |
| M16 | 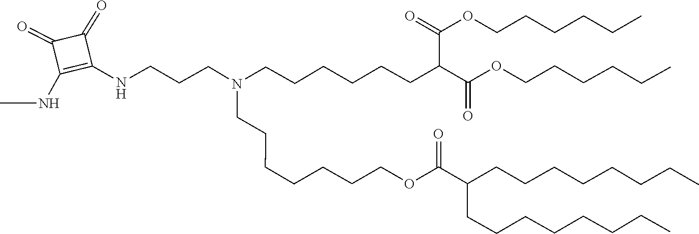 [SMILES: CCCCCCCCC(CCCCCCCC)C(=O)OCCCCCCCN(CCCCCCC(C(=O)OCCCCCC)C(=O)OCCCCCC)CCCNc1c(NC)c(=O)c1=O] |
| M17 | 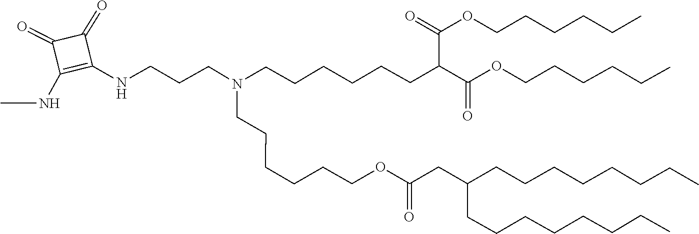 [SMILES: CCCCCCCCC(CCCCCCCC)CC(=O)OCCCCCCN(CCCCCCC(C(=O)OCCCCCC)C(=O)OCCCCCC)CCCNc1c(NC)c(=O)c1=O] |

and pharmaceutically acceptable salts thereof.

73. A lipid nanoparticle (LNP) comprising a compound of any one of claims 1-72, or a pharmaceutically acceptable salt thereof.

74. The LNP of claim 73, comprising about 40 mol % to about 60 mol %, inclusive, of the compound.

75. The LNP of claim 73 or 74, further comprising a phospholipid.

76. The LNP of claim 75, comprising about 1 mol % to about 20 mol %, inclusive, of the phospholipid.

77. The LNP of any one of claims 73-76, further comprising a structural lipid.

78. The LNP of claim 77, comprising about 30 mol % to about 50 mol %, inclusive, of the structural lipid.

79. The LNP of any one of claims 73-78, further comprising a PEG lipid.

80. The LNP of claim 79, comprising about 0.1 mol % to about 5 mol %, inclusive, of the PEG lipid.

81. An LNP comprising:
(i) a compound of any one of claims 1-72, or a pharmaceutically acceptable salt thereof;
(ii) a phospholipid;
(iii) a structural lipid; and
(iv) a PEG lipid.

82. The LNP of claim 81, comprising:
(i) about 40 mol % to about 60 mol %, inclusive, of the compound;
(ii) about 1 mol % to about 20 mol %, inclusive, of the phospholipid;
(iii) about 30 mol % to about 50 mol %, inclusive, of the structural lipid; and
(iv) about 0.1 mol % to about 5 mol %, inclusive, of the PEG lipid.

83. The LNP of any one of claims 75-82, wherein the phospholipid is selected from the group consisting of:
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanolamine (DOPE),
1,2-diphytanoyl-sn-glycero-3-phosphoethanolamine (ME 16.0 PE),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), and
sphingomyelin.

84. The LNP of claim 83, wherein the phospholipid is DSPC.

85. The LNP of any one of claims 77-84, wherein the structural lipid is a steroid.

86. The LNP of any one of claims 77-85, wherein the structural lipid is selected from the group consisting of cholesterol, fecosterol, sitosterol, ergosterol, campesterol, stigmasterol, and brassicasterol.

87. The LNP of claim 86, wherein the structural lipid is cholesterol.

88. The LNP of any one of claims 79-87, wherein the PEG lipid is a PEG-modified phosphatidylethanolamine, a PEG-modified phosphatidic acid, a PEG-modified ceramide, a PEG-modified dialkylamine, a PEG-modified diacylglycerol, or a PEG-modified dialkylglycerol.

89. The LNP of any one of claims 79-88, wherein the PEG lipid is of one of the following formulae: 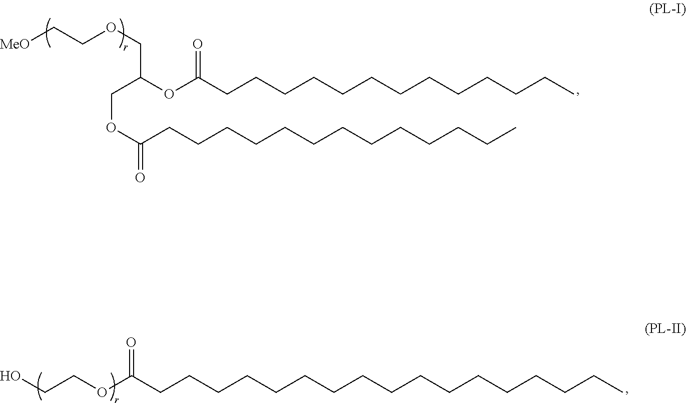
or a pharmaceutically accept salt thereof, wherein r is an integer from 1 to 100, inclusive.

90. The empty LNP of claim 89, wherein the PEG lipid is selected from: 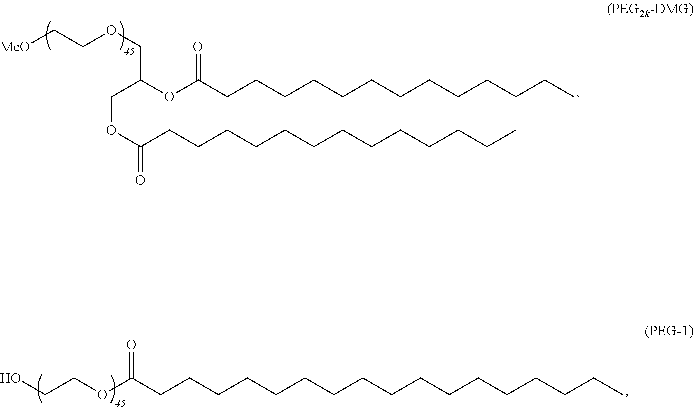
and pharmaceutically acceptable salts thereof.

91. The LNP of any one of claims 73-90, further comprising an active agent.

92. The LNP of claim 91, wherein the active agent is a nucleic acid.

93. The LNP of claim 92, wherein the nucleic acid is a short interfering RNA (siRNA), an asymmetrical interfering RNA (aiRNA), a RNA interference (RNAi) molecule, a microRNA (miRNA), an antagomir, an antisense RNA, a ribozyme, a Dicer-substrate RNA (dsRNA), a small hairpin RNA (shRNA), or a messenger RNA (mRNA).

94. The LNP of claim 93, wherein the nucleic acid is an mRNA.

95. A pharmaceutical composition comprising an LNP of any one of claim 73-94 and a pharmaceutically acceptable carrier.

96. A method of delivering an active agent to a cell of a subject comprising administering to the subject an LNP of any one of claims 73-94, or a pharmaceutical composition thereof, wherein the LNP comprises the active agent.

97. A method of specifically delivering an active agent to an organ of a subject comprising administering to the subject an LNP of any one of claims 73-94, or a pharmaceutical composition thereof, wherein the LNP comprises the active agent.

98. The method of claim 96 or 97, wherein the active agent is a nucleic acid.

99. The method of claim 98, wherein the nucleic acid is a short interfering RNA (siRNA), an asymmetrical interfering RNA (aiRNA), a RNA interference (RNAi) molecule, a microRNA (miRNA), an antagomir, an antisense RNA, a ribozyme, a Dicer-substrate RNA (dsRNA), a small hairpin RNA (shRNA), or a messenger RNA (mRNA).

100. The method of claim 99, wherein the nucleic acid is an mRNA.

101. A method of expressing a polypeptide in a cell of a subject comprising administering to the subject an LNP of any one of claims 73-94, or a pharmaceutical composition thereof, wherein the LNP comprises an mRNA encoding the polypeptide.

102. A method of treating and/or preventing a disease in a subject in need thereof, the method comprising administering to the subject an LNP of any one of claims 73-94.
